(19) United States
(12) Patent Application Publication
(10) Pub. No.: US 2008/0293647 A1
(43) Pub. Date: Nov. 27, 2008

(54) Combined Use Of Vitamin D Derivatives And Anti-Proliferative Agents For Treating Bladder Cancer

(76) Inventors: Luciano Adorini, Milan (IT); Giuseppe Penna, Cusano Milanino (IT)

Correspondence Address: EDWARDS ANGELL PALMER & DODGE LLP, P.O. BOX 55874, BOSTON, MA, 02205, US

(21) Appl. No.: 11/667,637

(22) PCT Filed: Nov. 11, 2005
(86) PCT No.: PCT/EP05/55931

§ 371 Date: Jan. 11, 2008

Related U.S. Application Data
(60) Provisional application No. 60/668,408, filed on Apr. 4, 2005.

(30) Foreign Application Priority Data
Nov. 12, 2004 (GB) 0424965.2

(51) Int. Cl. A61K 31/704; A61K 31/593; A61P 35/00

(52) U.S. Cl. 514/34; 514/167

(57) ABSTRACT

There is provided according to the invention a method of treating a patient with bladder cancer by administering a effective amount of a vitamin D compound in combination with one or more other antiproliferative agents. Also provided are uses of a vitamin D compound in combination with one or more other antiproliferative agents and compositions for use in the method.

### BACKGROUND OF THE INVENTION

Bladder cancer comprises several disease subtypes: transitional cell carcinoma CCC), squamous cell carcinoma (SCC), adenocarcinoma. Of these, TCC is predominant, accounting for 90% of all bladder carcinomas. Both TCC and SCC can be of a non-invasive or invasive type, and they are collectively known as superficial bladder cancer (see Schenkman & Lamm Scientific World Journal. 2004 (28):4 Suppl 1:387-99). Such cancers can be surgically removed (typically by transurethral resection), but have a great tendency for recurrence. The incidence of bladder cancer increases with age. People over the age of 70 develop the disease 2 to 3 times more often than those aged 55-69, and 15 to 20 times more often than those aged 30-54. Bladder cancer is 2 to 3 times more common in men compared to women. In the United States, approximately 38,000 men and 15,000 women are diagnosed with the disease each year. Bladder cancer is the fourth most common type of cancer in men and the eighth most common type in women.

As mentioned above, superficial bladder cancer can be surgically removed, but has a great tendency for recurrence (see Schenkman & Lamm Scientific World Journal. 2004 (28); 4 Suppl 1:387-99).

Anthracyclines such as doxorubicin and epirubicin are known for the treatment of bladder cancer. Administration of anthracyclines is believed to reduce the risk of recurrence following surgery however these compound have considerable systemic toxicity. Toxicity can be reduced by instilling the drug directly into the bladder, however it can still enter the system if the bladder has in any way been damaged by the surgery. Further these compounds are believed to be potentially carcinogenic irrespective of the route of administration. Thus, there is an unmet medical need for a safer treatment which prevents recurrence or progression to invasive disease.

As described herein, it has now surprisingly been found that vitamin D compounds when used in combination with one or more other anti-proliferative agents, for example an anthracycline compound such as doxorubicin or epirubicin, inhibit proliferation of bladder cancer cells and may therefore be expected to treat and prevent bladder cancer, in particular superficial bladder cancer.

Calcitriol (1,25 dihydroxycholecalciferol; the active, hormonal form of vitamin D) and its analogues can have significant anti-proliferative effects on various tumor cell lines, but clinical use as an anti-cancer agent can be limited by hypercalcemic liability. Superficial bladder cancer offers the unique advantage of being treatable by intravesical administration of anti-proliferative agents, thus permitting the use of high calcitriol concentrations while avoiding systemic side effects such as hypercalcemia and down-modulation of the anti-tumor immune response. Since studies in animal models have shown only marginal effects of intravesical calcitriol administration on superficial bladder cancer (see Konety et al. (2001) J. Urol. 165(1):253-258), the Inventors have examined the efficacy of combinations of calcitriol and various clinically used anti-proliferative agents.

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D_{3 [}25(OH)D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha,25(OH)₂D₃ (calcitriol) in a carefully regulated fashion (Fraser, D. R. and Kodicek, E. (1970) Nature 288:764-766; Wong, R. G. et al. (1972) J. Clin. Invest. 51:1287-1291), and the discovery of a nuclear receptor for 1-alpha,25(OH)₂D₃ (VDR) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as I-alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann. NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1-alpha,25(OH)₂D₃ to generate the requisite specific biological responses for this secosteroid hormone (Pike, J. W. (1991) Annu. Rev. Nutr. 11: 189-216). To date, there is evidence that nuclear receptors for 1-alpha,25(OH)₂D₃ (VDR) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors iii more than 30 tissues has led to the identification of vitamin D₃ as a pluripotent regulator outside its classical role in calcium/bone homeostasis. A paracrine role for 1-alpha,25(OH)₂D₃ has been suggested by the combined presence of enzymes capable of oxidizing vitamin D₃ into its active forms, e.g., 25-(OH)D-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Aim. Rev. Med. 40:71-78).

Given the activities of vitamin D₃ and its metabolites, much attention has focused on the development of synthetic analogues of these compounds. A large number of these analogues involve structural modifications in the A ring, B ring, C/D rings, and, primarily, the side chain (Bouillon, R. et al. (1995) Endocr. Rev. 16(2):200-257). Although a vast majority of the vitamin D₃ analogues developed to date involve structural modifications in the side chain, a few studies have reported the biological profile of A-ring diastereomers (Norman, A. W. et al. (1993) J. Biol. Chem. 268 (27):20022-20030). Furthermore, biological esterification of steroids has been studied (Hochberg, R. B. (1998) Endocr. Rev. 19(3): 331-348), and esters of vitamin D₃ are known (WO 97/11053).

Moreover, despite much effort in developing synthetic analogues, clinical applications of vitamin D and its structural analogues have been limited by the undesired side effects elicited by these compounds after administration to a subject for known indications/applications of vitamin D compounds.

The activated form of vitamin D, vitamin D₃, and some of its analogues have been described as potent regulators of cell growth and differentiation. It has previously been found that vitamin D₃, as well as an analogue (analogue V, referred to elsewhere herein as Compound B), inhibited Benign Prostatic Hyperplasia (BPH) cell proliferation and counteracted the mitogenic activity of potent growth factors for BPH cells, such as keratinocyte growth factor (KGF) and insulin-like growth factor (IGF1). Moreover, the analogue induced bcl-2 protein expression, intracellular calcium mobilization, and apoptosis in both unstimulated and KGF-stimulated BPH cells.

U.S. Pat. No. 5,939,408 and EP808833 disclose a number of 1,25(OH)₂D₃ analogues including the compound 1-alpha-fluoro-25-hydroxy-16,23E-diene-26,27-bishomo-20-epi-cholecalciferol (Compound A). U.S. Pat. No. 5,939,408 and EP808833 disclose that the compounds induce differentiation and inhibition of proliferation in various skin and cancer cell lines and are useful for the treatment of hyperproliferative skin diseases such as psoriasis, neoplastic diseases such a leukemia, breast cancer and sebaceous gland diseases such as acne and seborrheic dermatitis and osteoporosis.

### BRIEF DESCRIPTION

The present invention is further described below with reference to the following non-limiting examples and with reference to the following figures, in which:

FIG. 1 shows synergistic or antagonistic effects on bladder cancer cell proliferation by chemotherapeutic agents in combination with calcitriol.

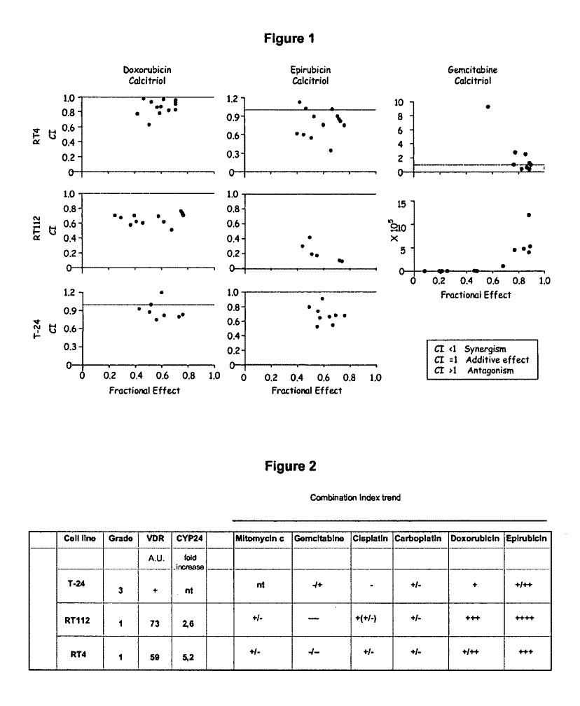

FIG. 2 shows combination index values, for combined treatments with calcitriol and chemotherapeutic agents in the in vitro inhibition of human bladder cancer cell line proliferation.

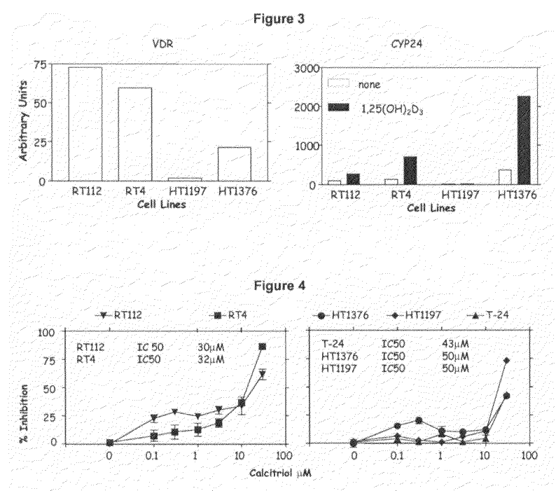

FIG. 3 shows VDR expression and up-regulation of CYP24 in human bladder cancer cell line incubated with calcitriol.

FIG. 4 shows inhibition of bladder cancer cell line proliferation by calcitriol.

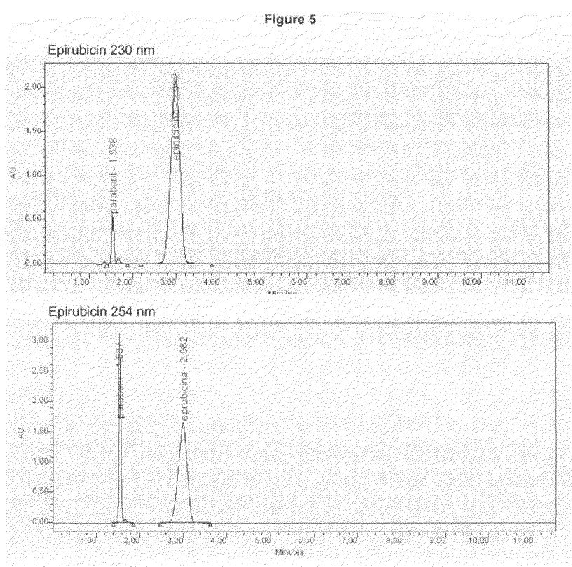

FIG. 5 shows the UV chromatograms from detection at 230 nm and 254 nm respectively for epirubicin alone in the compatibility study described in Example 50.

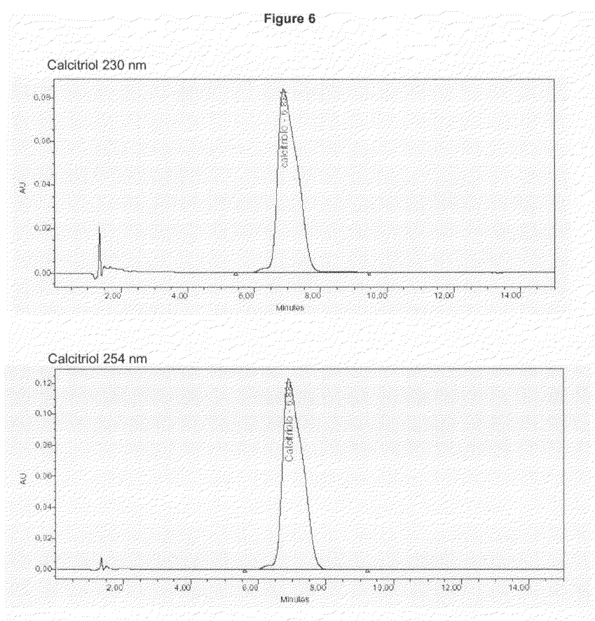

FIG. 6 shows the UV chromatograms from detection at 230 nm and 254 nm respectively for calcitriol alone in the compatibility study described in Example 50.

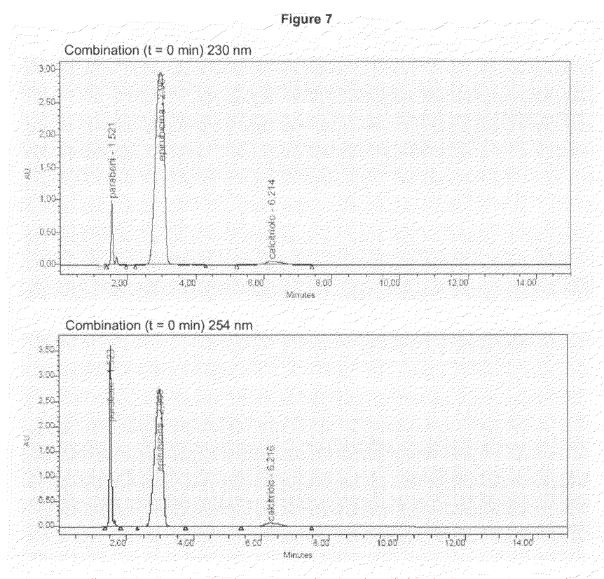

FIG. 7 shows the UV chromatograms from detection at 230 nm and 254 nm respectively for epirubicin and calcitriol combination at t=0 in the compatibility study described in Example 50.

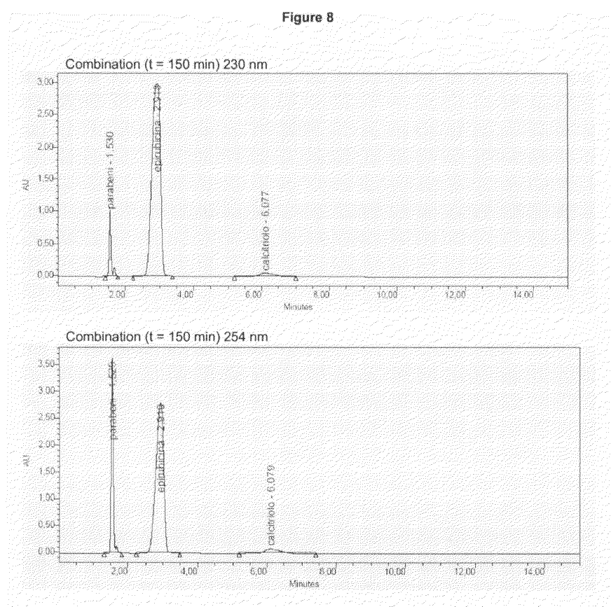

FIG. 8 shows the UV chromatograms from detection at 230 nm and 254 nm respectively for epirubicin and calcitriol combination at t=150 in the compatibility study described in Example 50.

### SUMMARY OF THE INVENTION

The Inventors have now surprisingly found, as demonstrated in the Examples herein, that vitamin D analogues, such as calcitriol, when used in combination with one or more other anti-proliferative agents, for example doxorubicin or epirubicin, are effective in inhibiting proliferation of bladder cancer cells and may therefore be expected to be useful in treating bladder cancer.

More particularly there appears to be a synergistic interaction between the vitamin D compound and the other anti-proliferative agents which offers the potential to reduce achieve greater efficacy with a given dose of other anti-proliferative agent or to achieve a similar efficacy with a lower dose of other anti-proliferative agent. This would be most beneficial to patients in view of the toxicity issues with anti-proliferative agents such as the anthracyclines.

The vitamin D compound used may, for example, be calcitriol, or a vitamin D3 analogue, for example 1-alpha-fluoro-25-hydroxy-16,23E-diene-26,27-bishomo-20-epi-cholecalciferol. Other vitamin D compounds include those in Table 1 of Example 51.

The anti-proliferative agent may be a cytostatic agent, for example, an anthracycline compound such as doxorubicin, epirubicin, daunorubicin, aclarubicin, idarubicin, pirarubicin, annamycin, methoxymorpholinodoxorubicin, cyanomorpholinyl doxorubicin, valrubicin (N-trifluoroacetyladriamycin-14-valerate), or mitoxantrone and other anthracyclines or analogues thereof, respectively.

Thus the invention provides the use of a vitamin D compound in combination with one or more other anti-proliferative agents, for example an anthracycline, in new methods of treatment using such compounds, for the prevention or treatment of bladder cancer, and associated symptoms.

Thus the invention provides vitamin D compounds in combination with one or more other anti-proliferative agents, for example an anthracycline, and new methods of treatment using such combinations, for the prevention or treatment of bladder cancer. More particularly, the invention provides the use of vitamin D compounds in combination with one or more other anti-proliferative agents for the manufacture of a medicament for the prevention and/or treatment of bladder cancer.

The invention also provides a method for preventing and/or treating bladder cancer, by administering a vitamin D compound in combination with one or more other anti-proliferative agents, for example an anthracycline, in amounts effective to prevent and/or to treat bladder cancer.

The invention still further provides a kit containing a vitamin D compound together with instructions directing administration of the vitamin D compound in combination with one or more other anti-proliferative agents, for example an anthracycline to a patient in need of prevention or treatment of bladder cancer thereby to prevent or treat bladder cancer in said patient.

### DETAILED DESCRIPTION OF THE INVENTION

### I. Definitions and Discussion

Before further description of the present invention, and in order that the invention may be more readily understood, certain terms are first defined and collected here for convenience.

By “bladder cancer” it is meant a malignant tumor growth within the bladder. Bladder cancers usually arise from the transitional cells of the bladder (the cells lining the bladder). Bladder cancer comprises several disease subtypes: transitional cell carcinoma (TCC), squamous cell carcinoma (SCC), adenocarcinoma. Both TCC and SCC can be of a non-invasive or invasive type, and they are collectively known as superficial bladder cancer. Whilst not wishing to be bound by theory the inventors believe that the invention is particularly able to treat superficial bladder cancer. There are three recognised ‘substages’ of superficial bladder tumors:
Ta-papillary tumor confined to the urothelium (inner lining of the bladder)
T1-papillary tumor invading the underlying lamina propria
Tcis (also written as TIS, CIS, carcinoman in situ)-flat-reddened lesion with high grade histologic features confined to the urothelium
   to which the methods, compositions and kits of the invention are applicable.

The term “other anti-proliferative agent” refers to anti-proliferative agents other than a vitamin D compound (which can exhibit anti-proliferative properties itself). Generally the anti-proliferative agent of the invention is a cytostatic agent. Preferably, the anti-proliferative agent is an anthracycline compound such as doxorubicin, epirubicin, daunorubicin, aclarubicin, idarubicin, pirarubicin, annamycin, methoxymorpholinodoxorubicin, cyanomorpholinyl doxorubicin, valrubicin (N-trifluoroacetyladriamycin-14-valerate), or mitoxantrone and other anthracyclines or analogues thereof, respectively. Another example is epidaunomycin. One anti-proliferative compound of particular interest is doxorubicin. Doxorubicin has been used for a long time in the antineoplastic treatment, for a review see Arcamone, ed. “Doxorubicin”, Acad. Press, New York 1981. A serious side-effect of doxorabicin is the onset of often irreversible myocardiopathies. Another anti-proliferative compound of particular interest is epirubicin. Epirubicin was found to have advantageous pharmacological properties compared with doxorubicin, showing an equivalent antitumoral activity but less side-effects (R. B. Weiss et al., Cancer Chemother. Pharmacol. 18, 185-97 (1986)). The anti-proliferative agent may also be liposomal in formulation, for example liposomal doxorubicin. Also considered within the scope of the invention are other anthracycline compounds as known in the art, for example, those described by Farquhar et al. J Med Chem. (1998) 41(6):965-72, Rho et al. Bull. Korean Chem. Soc. (2001) 22(9):963-968, WP744, WP769, WP631 as described by Inge et al. J Surg Res. (2004) 121(2):187-96 and MEN-10755 as described by Bos et al. Cancer Chemother Pharmacol. (2004) 54(1):64-70.

Further references in connection with the activity of anthracycline compounds include: Doxorubicin. Anticancer Antibiotics, Federico Arcamone, 1981, Publ: Academic Press, New York, N.Y.; Adriamycin Review, EROTC International Symposium Brussels, May, 1974, edited by M. Staquet, Publ. Eur. Press Medikon, Ghent, Belg.; and Results of Adriamycin Therapy, Adriamycin Symposium at Frankfurt/Main 1974 edited by M. Ghione, J. Fetzer and H. Maier, publ.: Springer, New York, N.Y.

The term “administration” or “administering” includes routes of introducing the vitamin D compound(s) to a subject to perform their intended function. Examples of routes of administration which can be used include injection (subcutaneous, intravenous, parenterally, intraperitoneally), oral, inhalation, rectal, transdermal or in a preferred embodiment of the invention, via bladder (intravesical) instillation.

The pharmaceutical preparations are, of course, given by forms suitable for each administration route. For example, the preparations may be administered orally in tablets or capsule form, by injection, inhalation, topically as a lotion or ointment, rectally as a suppository etc. The injection can be bolus or can be continuous infusion. Depending on the route of administration, the vitamin D compound can be coated with or disposed in a selected material to protect it from natural conditions which may detrimentally effect its ability to perform its intended function. The vitamin D compound is administered in conjunction with a cytostatic agent, for example, an anthracycline compound such as doxorubicin, epirubicin, daunorubicin, aclarubicin, idarubicin, pirarubicin, annamycin or mitoxantrone and other anthracyclines or analogues thereof, respectively or with a pharmaceutically-acceptable carrier, or both. The vitamin D compound can be administered prior to the administration of the anti-proliferative agent(s), simultaneously with the anti-proliferative agent(s), or after the administration of the anti-proliferative agent(s). The vitamin D compound can be administered in a combined preparation with the one or more anti-proliferative agents or else the vitamin D compound and the one or more anti-proliferative agents may be administered in separate preparations. The vitamin D compound can be administered by the same route as the one or more anti-proliferative agents or else the vitamin D compound and the one or more anti-proliferative agents may be administered by different routes. Furthermore, the vitamin D compound can also be administered in a pro-form which is converted into its active metabolite, or more active metabolite in vivo.

The combination of a Vitamin D compound and one or more other anti-proliferative agents may be co-administered, simultaneously or sequentially with other known treatments for bladder cancer, for example, intravesical BCG Immunotherapy (see Scheizkman & Lamm ScientificWorldJournal. 2004 (28);4 Suppl 1:387-99.)

The term “effective amount” includes an amount effective, at dosages and for periods of time necessary, to achieve the desired result, i.e. sufficient to treat bladder cancer. An effective amount of vitamin D compound and/or anti-proliferative agent may vary according to factors such as the disease state, age and weight of the subject, and the ability of the vitamin D compound and/or anti-proliferative agent to elicit a desired response in the subject. Dosage regimens may be adjusted to provide the optimum therapeutic response. An effective amount is also one in which any toxic or detrimental effects (e.g., side effects) of the vitamin D compound and/or anti-proliferative agent are outweighed by the therapeutically beneficial effects.

The term “treat” or “treatment” means causing an anti-neoplastic effect including, for example, one more of the following effects: inhibition of proliferation associated with cell cycle arrest and induction of apoptosis, induction of cell differentiation, reduction in invasiveness and inhibition of angiogenesis. The term ‘treat’ will also be understood to include use subsequent to surgery to reduce the risk of recurrence of bladder cancer.

Those skilled in the art will recognise that the vitamin D compound may be used in human or veterinary medicine. It is preferred that the vitamin D compound be used in the treatment of human patients.

A therapeutically effective amount of vitamin D compound (i.e., an effective dosage) may range from about 0.001 to 30 ug/kg body weight, preferably about 0.01 to 25 ug/kg body weight, more preferably about 0.1 to 20 ug/kg body weight, and even more preferably about 1 to 10 ug/kg, 2 to 9 ug/kg, 3 to 8 ug/kg, 4 to 7 ug/kg, or 5 to 6 ug/kg body weight. The skilled artisan will appreciate that certain factors may influence the dosage required to effectively treat a subject, including but not limited to the severity of the disease or disorder, previous treatments, the general health and/or age of the subject, and other diseases present. In addition, the dose administered will also depend on the particular vitamin D compound used, the effective amount of each compound can be determined by titration methods known in the art. Moreover, treatment of a subject with a therapeutically effective amount of a vitamin D compound can include a single treatment or, preferably, can include a series of treatments. In one example, a subject is treated with a vitamin D compound in the range of between about 0.1 to 20 ug/kg body weight, once per day for a duration of six months or longer, for example for life depending on management of the symptoms and the evolution of the condition. Also, as with other chronic treatments an “on-off” or intermittent treatment regime can be considered. It will also be appreciated that the effective dosage of a vitamin D compound used for treatment may increase or decrease over the course of a particular treatment.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorus atoms replacing one or more carbons of the hydrocarbon backbone. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer e.g., 1-6 carbon atoms, such as 1-4 carbon atoms. Example alkyl groups include methyl, ethyl, propyl (e.g. n-propyl, i-propyl) and butyl (e.g. n-butyl, i-butyl, t-butyl). Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an allyl substituted with an aryl (e.g., phenylmethyl (benzyl)). The term “alkyl” also includes unsaturated aliphatic groups analogous in length and possible substitution to the alkyls described above, but that contain at least one double or triple bond.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiments, the term “lower alkyl” includes a straight chain alkyl having 4 or fewer carbon atoms in its backbone, e.g., C₁-C₄ alkyl.

The terms “alkoxyalkyl”, “polyaminoalkyl” and “thioalkoxyalkyl” refer to alkyl groups, as described above, which further include oxygen, nitrogen or sulfur atoms replacing one or more carbons of the hydrocarbon backbone.

The term “aryl” as used herein, refers to the radical of aryl groups, including 5- and 6-membered single-ring aromatic groups that may include from zero to four heteroatoms selected e.g., from, O, N and S, for example, benzene, pyrrole, furan, thiophene, imidazole, triazole, tetrazole, pyrazole, pyridine, pyrazine, pyridazine and pyrimidine, and the like. Aryl groups also include polycyclic fused aromatic groups (preferably 9 or 10 membered) such as naphthyl, quinolyl, indolyl, and the like. Further examples include benzoxazole and benzothiazole. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g. tetralin).

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “chiral” refers to molecules which have the property of non-superimposability of the mirror image partner, while the term “achiral” refers to molecules which are superimposable on their mirror image partner.

The term “isomers” or “stereoisomers” refers to compounds which have identical chemical constitution, but differ with regard to the arrangement of the atoms or groups in space.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

As used herein, the term “halogen” designates —F, —Cl, —Br or —I; the term “sulfhydryl” or “thiol” means —SH; the term “hydroxyl” means —OH.

The term “haloalkyl” is intended to include alkyl groups as defined above that are mono-, di- or polysubstituted by halogen, e.g., fluoroalkyl such as fluoromethyl and trifluoromethyl.

The term “hydroxyalkyl” is intended to include alkyl groups as defined above that are mono-, di- or polysubstituted by hydroxy, e.g., hydroxymethyl or 2-hydroxyethyl.

The term “heteroatom” as used herein means an atom of any element other than carbon or hydrogen. Preferred heteroatoms are nitrogen, oxygen, sulfur and phosphorus especially N, O and S.

As used herein, “instill,” “instilled,” “instillation,” refers to one or more of the following; to drop in, to pour in drop by drop, to impart gradually, to infuse slowly (e.g. example infuse slowly an intravesical solution).

As used herein, “intravesical,” refers to inside the bladder. As such, “intravesical instillation,” “intravesical therapy,” “instill,” and “instillation” refers to solutions that are administered directly into the bladder. In some embodiments, instillation is via catheterization. Further, “intravesical solution,” “intravesical agent,” “intravesical therapeutic,” and intravesical compound” refers to a treatment that can be administered to the bladder. For example, in one embodiment, an intravesical agent is a vitamin D receptor agonist, for example calcitriol. In another embodiment, an intravesical agent is an anthracycline, for example doxorubicin or epirubicin. In one embodiment, intravesical therapy is a combination of an oral and an intravesical agent. It is not intended that the present invention be limited to a combination of an oral and an intravesical agent. For example, in one embodiment, intravesical therapy is an intravesical agent. In another embodiment, intravesical therapy is a combination of intravesical agents e.g. calcitriol and either doxorubicin or epirubicin.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The terms “isolated” or “substantially purified” are used interchangeably herein and refer to vitamin D compounds (e.g., vitamin D₃ compounds) in a non-naturally occurring state.

The compounds can be substantially free of cellular material or culture medium when naturally produced, or chemical precursors or other chemicals when chemically synthesized. In certain preferred embodiments, the terms “isolated” or “substantially purified” also refer to preparations of a chiral compound which substantially lack one of the enantiomers; i.e., enantiomerically enriched or non-racemic preparations of a molecule. Similarly, the terms “isolated epimers” or “isolated diastereomers” refer to preparations of chiral compounds which are substantially free of other stereochemical forms. For instance, isolated or substantially purified vitamin D₃ compounds include synthetic or natural preparations of a vitamin D₃ enriched for the stereoisomers having a substituent attached to the chiral carbon at position 3 of the A-ring in an alpha-configuration, and thus substantially lacking other isomers having a beta-configuration. Unless otherwise specified, such terms refer to vitamin D₃ compositions in which the ratio of alpha to beta forms is greater than 1:1 by weight. For instance, an isolated preparation of an alpha-epimer means a preparation having greater than 50% by weight of the alpha-epimer relative to the beta-epimer more preferably at least 75% by weight, and even more preferably at least 85% by weight. Of course the enrichment can be much greater than 85%, providing “substantially epimer-enriched” preparations, i.e., preparations of a compound which have greater than 90% of the alpha-epimer relative to the beta-stereoisomer, and even more preferably greater than 95%. The term “substantially free of the beta stereoisomer” will be understood to have similar purity ranges.

As used herein, the term “vitamin D compound” includes any compound being vitamin D or an analogue thereof that is capable of treating or preventing bladder cancer. Generally, compounds which are ligands for the vitamin D receptor (VDR ligands) and which are capable of treating or preventing bladder cancer are considered to be within the scope of the invention. Vitamin D compounds are preferably agonists of the vitamin D receptor. Thus, vitamin D compounds are intended to include secosteroids. Examples of specific vitamin D compounds suitable for use in the methods of the present invention are further described herein. A vitamin D compound includes vitamin D₂ compounds, vitamin D₃ compounds, isomers thereof, or derivatives/analogues thereof. Preferred vitamin D compounds are vitamin D₃ compounds which are ligands of (more preferably are agonists of the vitamin D receptor. Preferably the vitamin D compound (e.g., the vitamin D₃ compound) is a more potent agonist of the vitamin D receptor than the native ligand (i.e. the vitamin D, e.g., vitamin D₃). Vitamin D₁ compounds, vitamin D₂ compounds and vitamin D₃ compounds include, respectively, vitamin D₁, D₂, D₃ and analogues thereof. Other examples of vitamin D compounds include those in Table 1 of Example 51.

In certain embodiments, the vitamin D compound may be a steroid, such as a secosteroid, e.g., calciol, calcidiol or calcitriol.

The term “secosteroid” is art-recognized and includes compounds in which one of the cyclopelitanoperhydro-phenanthrene rings of the steroid ring structure is broken. For example, 1-alpha,25(OH)₂D₃ and analogues thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trains conformer of 1-alpha,25(OH)₂D₃ is illustrated herein having all carbon atoms numbered using standard steroid notation.

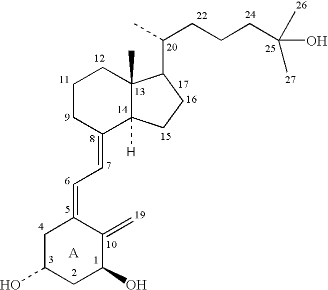

In the formulas presented herein, the various substituents on ring A are illustrated as joined to the steroid nucleus by one of these notations: a dotted line (----) indicating a substituent which is in the beta-orientation (i.e., above the plane of the ring), a wedged solid line ( [symbol] ) indicating a substituent which is in the alpha-orientation (i.e., below the plane of the molecule), or a wavy line ( [symbol] ) indicating that a substituent may be either above or below the plane of the ring. In regard to ring A, it should be understood that the stereochemical convention in the vitamin D field is opposite from the general chemical field, wherein a dotted line indicates a substituent on Ring A which is in an alpha-orientation (i.e., below the plane of the molecule), and a wedged solid line indicates a substituent on ring A which is in the beta-orientation (i.e., above the plane of the ring).

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “chiral carbon centers.” Regardless, both configurations, cis/trans and/or Z/E are contemplated for the compounds for use in the present invention.

With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

Also, throughout the patent literature, the A ring of a vitamin D compound is often depicted in generic formulae as any one of the following structures:

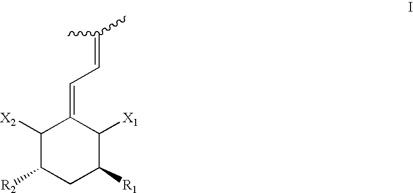

wherein X₁ and X₂ are defined as H or ═CH₂; or

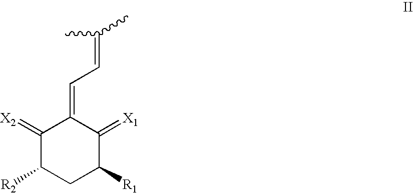

wherein X₁ and X₂ are defined as H₂ or CH₂.

Although there does not appear to be any set convention, it is clear that one of ordinary skill in the art understands either formula I or II to represent an A ring in which, for example, X₁ is ═CH₂ and X₂ is defined as H₂, as follows:

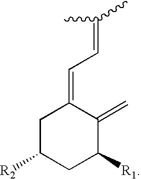

For purposes of the instant invention, formula II will be used in all generic structures.

Thus, in one aspect, the invention provides the use of a vitamin D compound in combination with one or more other anti-proliferative agents in the prevention or treatment of bladder cancer. It provides a vitamin D compound in combination with one or more other anti-proliferative agents for use in the prevention or treatment of bladder cancer. Also provided is a method of treating a patient with bladder cancer or preventing bladder cancer by administering an effective amount of a vitamin D compound in combination with one or more other anti-proliferative agents. More particularly, there is provided a method of prevention or treatment of bladder cancer in a patient in need thereof by administering an effective amount of a vitamin D compound in combination with one or more other anti-proliferative agents thereby to prevent or treat bladder cancer in said patient. Said method typically further comprises the step of obtaining or synthesising the vitamin D compound. The vitamin D compound is usually formulated in a pharmaceutical composition together with a pharmaceutically acceptable diluent or carrier. The one or more other anti-proliferative agents are usually formulated in a pharmaceutical composition together with a pharmaceutically acceptable diluent or carrier. The vitamin D compound and the one or more anti-proliferative agents may be formulated separately or together in a pharmaceutical composition together with a pharmaceutically acceptable diluent or carrier. Further provided is the use of a vitamin D compound in combination with one or more other anti-proliferative agents in the manufacture of a medicament for the prevention or treatment of bladder cancer. There is also provided a kit containing a vitamin D compound together with instructions directing administration of the vitamin D compound and one or more anti-proliferative agents to a patient in need of prevention or treatment of bladder cancer thereby to prevent or treat bladder cancer in said patient, especially wherein the vitamin D compound and the one or more anti-proliferative agents are formulated in a pharmaceutical composition together with a pharmaceutically acceptable diluent or carrier.

In one embodiment, the vitamin D compound for use in accordance with the invention comprises a compound of formula I:

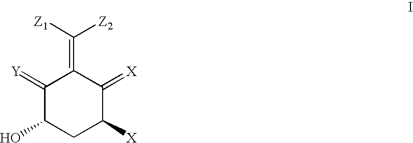

wherein
X is hydroxyl or fluoro;

### Y is H₂ or CH₂;

Z₁ and Z₂ are H or a substituent represented by formula II, provided Z₁ and Z₂ are different:

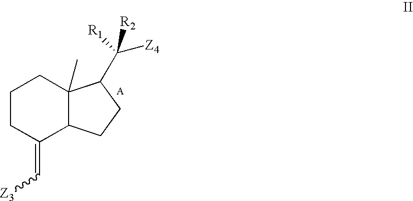

wherein
Z₃ represents the above-described formula I;
A is a single bond or a double bond;
R₁, R₂, and Z₄, are each, independently, hydrogen, alkyl, or a saturated or unsaturated carbon chain represented by formula III, provided that at least one of R₁, R₂, and Z₄ is the saturated or unsaturated carbon chain represented by formula III and provided that all of R₁, R₂, and Z₄ are not a saturated or unsaturated carbon chain represented by formula III:

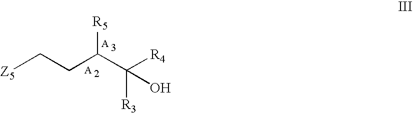

wherein
Z₅ represents the above-described formula II;
A₂ is a single bond, a double bond, or a triple bond;
A₃ is a single bond or a double bond; and
R₃, and R₄, are each, independently, hydrogen, alkyl, haloalkyl, hydroxyalkyl; and R₅ is hydrogen, H₂ or oxygen.

Thus, in the above structure (and in corresponding structures below), when A₂ represents a triple bond R₅ is absent. When A₂ represents a double bond R₅ represents hydrogen. When A₂ represents a single bond R₅ represents a carbonyl group or two hydrogen atoms.

In another embodiment, the vitamin D compound for use in accordance with the invention is a compound of formula:

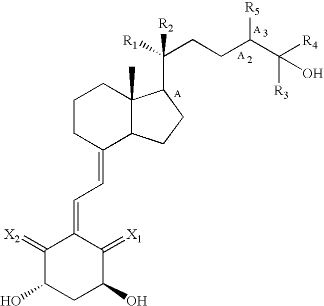

wherein:
X₁ and X₂ are H₂ or CH₂, wherein X₁ and X₂ are not CH₂ at the same time;
A is a single or double bond;
A₂ is a single, double or triple bond;
A₃ is a single or double bond;
R₁ and R₂ are hydrogen, C₁-C₄ alkyl or 4-hydroxy-4-methylpentyl, wherein R₁ and R₂ are not both hydrogen;
R₅ is hydrogen, H₂ or oxygen;
R₃ is C₁-C₄ alkyl, hydroxyalkyl or haloalkyl, e.g., fluoroalkyl, e.g., fluoromethyl or trifluoromethyl; and
R₄ is C₁-C₄ alkyl, hydroxyalkyl or haloalkyl, e.g. fluoroalkyl, e.g., fluoromethyl or trifluoromethyl.
For example, R₁ and R₂ may represent hydrogen or C₁-C₄ alkyl wherein R₁ and R₂ are not both hydrogen;

An example compound of the above structure is 1,25-dihydroxy-16-ene-23-yne cholecalciferol (elsewhere referred to herein as “Compound B”).

In yet another embodiment, the vitamin D compound for use in accordance with the invention is a “gemini” compound of the formula:

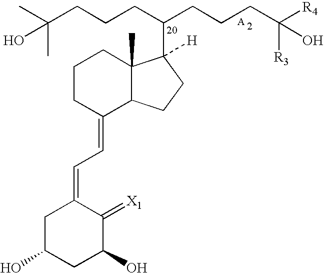

wherein:

### X₁ is H₂ or CH₂;

A₂ is a single, a double or a triple bond;
R₃ is C₁-C₄ alkyl, hydroxyalkyl, or haloalkyl, e.g., fluoroalkyl, e.g., fluoromethyl or trifluoromethyl;
R₄ is C₁-C₄ alkyl, hydroxyalkl or haloalkyl, e.g., fluoroalkyl, e.g., fluoromethyl or trifluoromethyl; and
the configuration at C₂₀ is R or S.

An example gemini compound of the above structure is 1,25-dihydroxy-21-(3-hydroxy-3-methylbutyl)-19-nor-cholecalciferol:

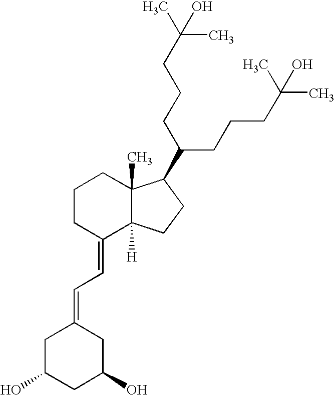

The synthesis of this compound is described in WO98/49138 which is herein incorporated in its entirety by reference.

In another embodiment, the vitamin D compound for use in accordance with the invention is a compound of the formula:

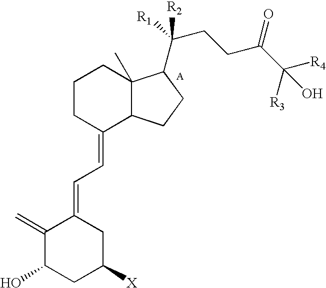

wherein:
A is a single or double bond;
R₁ and R₂ are each, independently, hydrogen or alkyl e.g., methyl;
R₃, and R₄, are each, independently, alkyl; and
X is hydroxyl or fluoro.

In a further embodiment, the vitamin D compound for use in accordance with the invention is a compound having the formula:

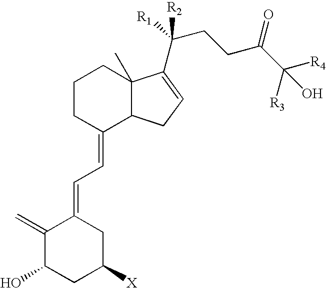

wherein:
R₁ and R₂, are each, independently, hydrogen, or alkyl, e.g., methyl;
R₃ is alkyl, e.g., methyl,
R₄ is alkyl, e.g., methyl; and
X is hydroxyl or fluoro.

In specific embodiments of the invention, the vitamin D compound for use in accordance with the invention is selected from the group consisting of:

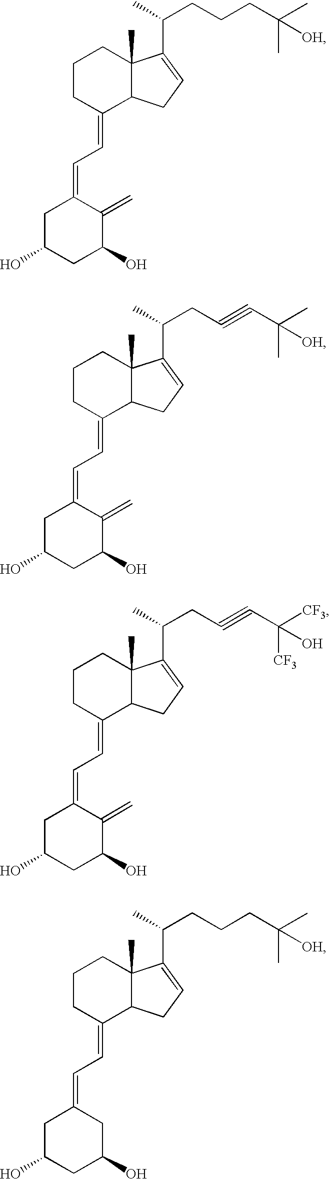

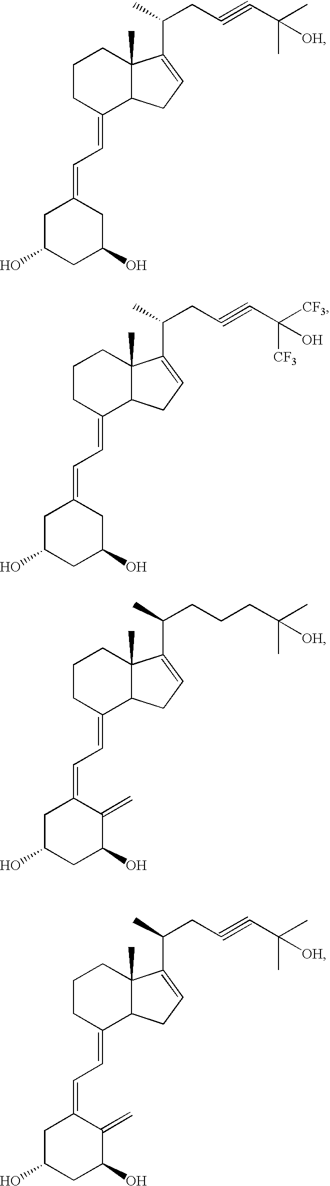

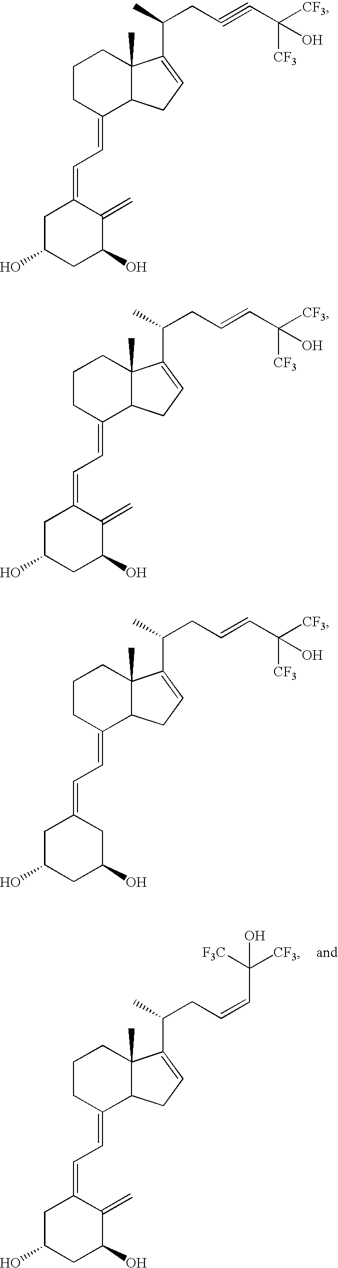

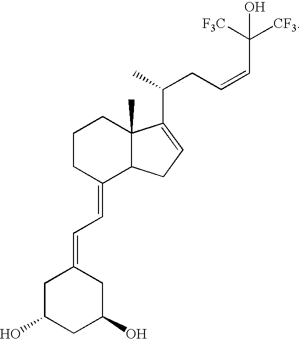

In other specific embodiments of the invention, the vitamin D compound for use in accordance with the invention is selected front the group consisting of:

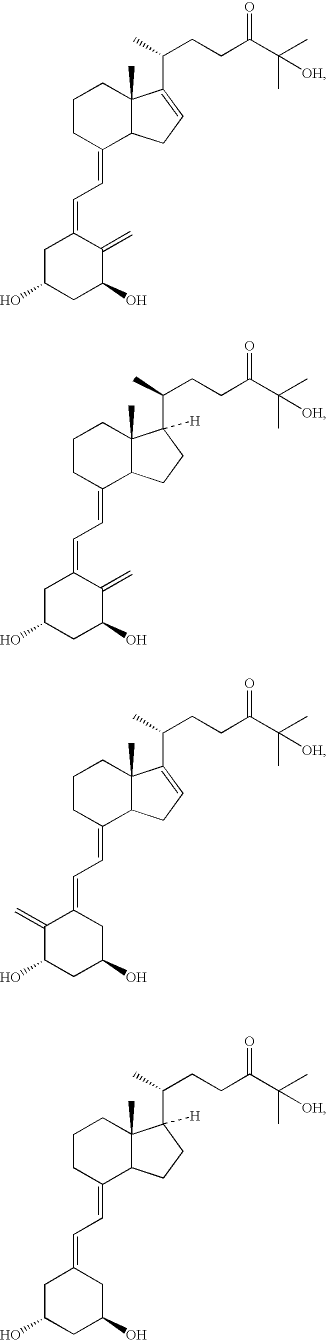

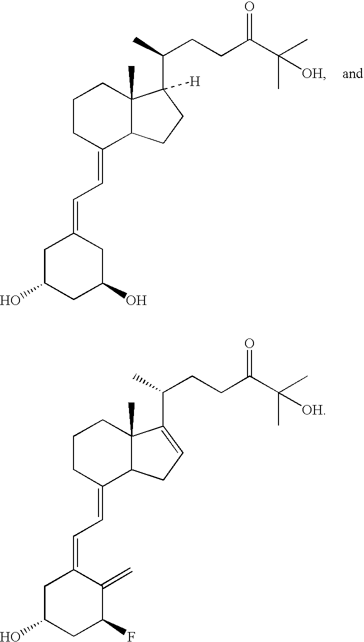

In further specific embodiments, the vitamin D compound for use in accordance with the invention is selected from the group of gemini compounds consisting of:

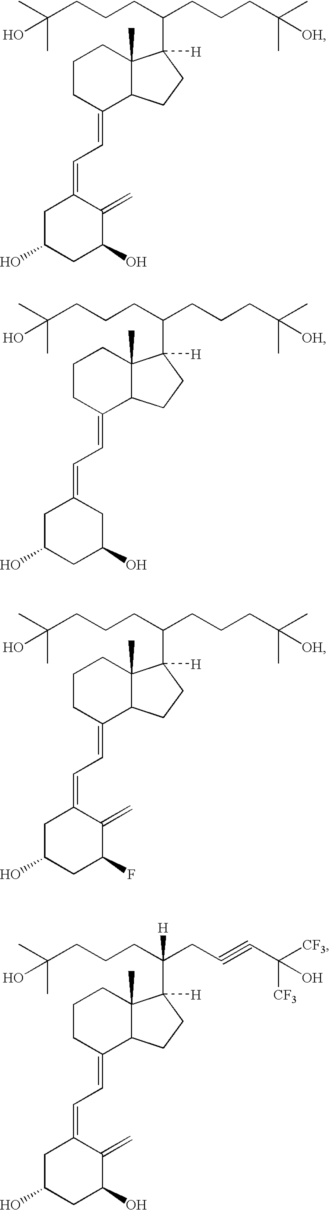

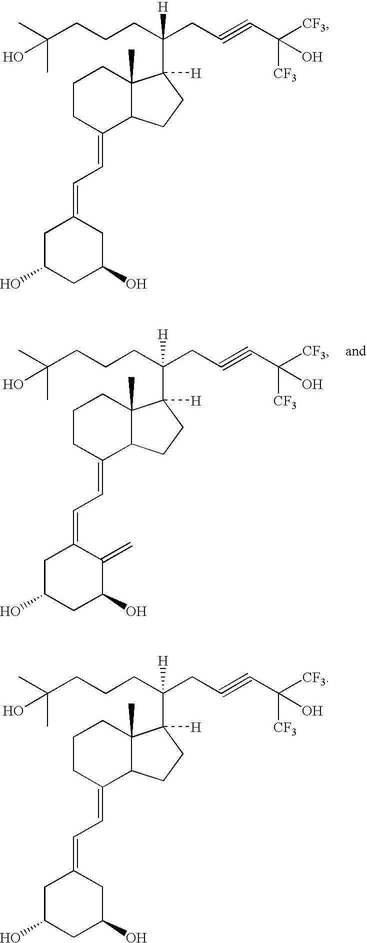

In still further specific embodiments of the invention, the vitamin D compound for use in accordance with the invention is a “Gemini” compound of the formula:

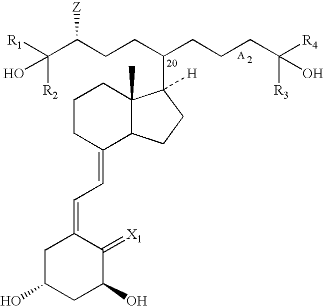

wherein:

### X₁ is H₂ or CH₂;

A₂ is a single, a double or a triple bond;
R₁, R₂, R₃ and R₄ are each independently C₁-C₄ alkyl, hydroxyalkyl, or haloalkyl, e.g., fluoroalkyl, e.g., fluoromethyl or trifluoromethyl;

### Z is —OH, —O, —NH₂ or —SH;

the configuration at C₂₀ is R or S;
and pharmaceutically acceptable esters, salts, and prodrugs thereof.

Compounds of this formula may be referred to as “geminal vitamin D₃” compounds due to the presence of two alkyl chains at C20.

Z may typically represent —OH.

In a further embodiment, X₁ is CH₂. In another embodiment, A₂ is a single bond. In another, R₁, R₂, R₃, and R₄ are each independently methyl or ethyl. In a further embodiment, Z is —OH. In an example set of compounds, X₁ is CH₂; A₂ is a single bond; R₁, R₂, R₃, and R₄ are each independently methyl or ethyl; and Z is —OH. In an even further embodiment, R₁, R₂, R₃, and R₄ are each methyl.

In a further embodiment of the invention, the vitamin D compound for use in accordance with the invention is a gemini compound of the formula:

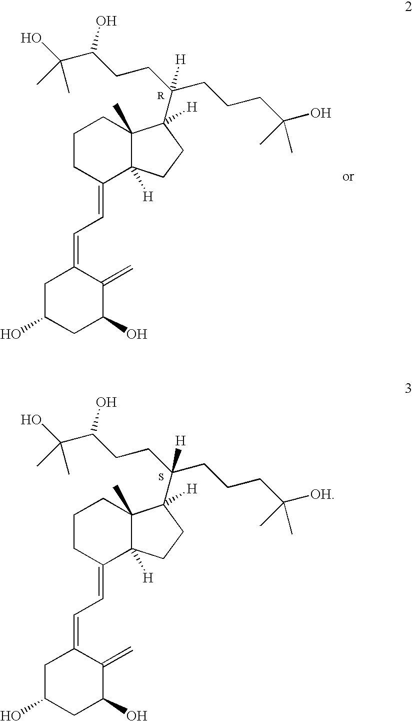

The chemical names of the compounds 2 and 3 mentioned above are:
1,25-dihydroxy-21-(2R,3-dihydroxy-3-methyl-butyl)-20R-cholecalciferol; and
1,25-dihydroxy-21-(2R,3-dihydroxy-3-methyl-butyl)-20S-cholecalciferol.

Additional embodiments of gemini compounds include the following vitamin D compounds for use in accordance with the invention.

### 1,25-Dihydroxy-21.-(2R,3-dihydroxy-3-methyl-butyl)-20S-19-nor-cholecalciferol

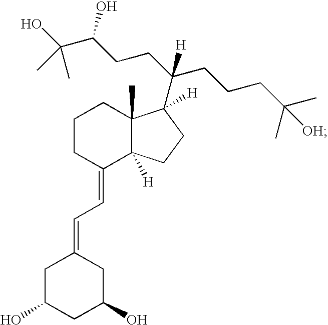

### 1,25-Dihydroxy-20S-21-(3-hydroxy-3-methyl-butyl)-24-keto-19-nor-cholecalciferol

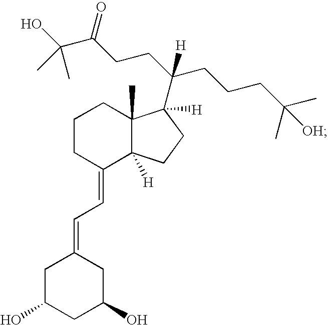

### 1,25-Dihydroxy-20S-21-(3-hydroxy-3-methyl-butyl)-24-keto-cholecalciferol

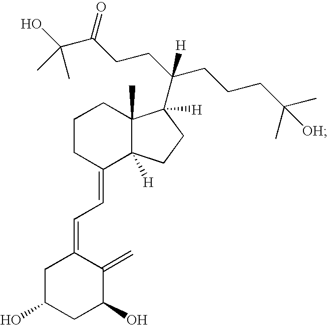

### 1,25-Dihydroxy-21(3-hydroxy-3-trifluoromethyl-4-trifluoro-butynyl)-26,27-hexadeutero-19-nor-20S-cholecalciferol

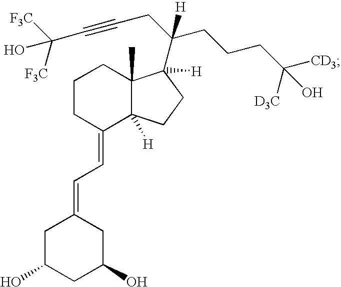

and

### 1,25-Dihydroxy-21(3-hydroxy-3-trifluoromethyl-4-trifluoro-butynyl)-26,27-hexadeutero-20S-cholecalciferol

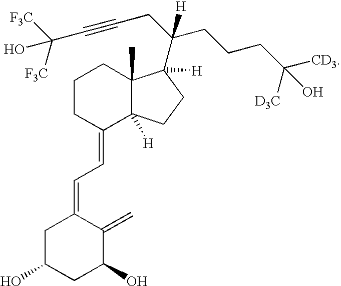

In further embodiments of the invention, the vitamin D compound for use in accordance with the invention is a compound of the formula:

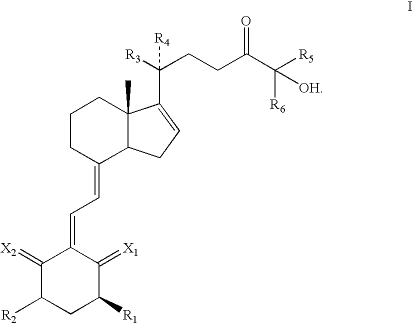

wherein:
X₁ and X₂ are each independently H₂ or CH₂, provided X₁ and X₂ are not both —CH₂;
R₁ and R₂ are each independently hydroxyl, OC(O)C₁-C₄ alkyl, OC(O)hydroxyalkyl or OC(O)fluoroalkyl;
R₃ and R₄ are each independently hydrogen, C₁-C₄ alkyl, hydroxyalkyl or haloalkyl or R₃ and
R₄ taken together with C₂₀ form C₃-C₆ cycloalkyl; and
R₅ and R₆ are each independently C₁-C₄ alkyl, hydroxyalkyl or haloalkyl; and pharmaceutically acceptable esters, salts, and prodrugs thereof.

R₃ and R₄ will preferably each be independently selected from hydrogen and C₁-C₄ alkyl.

In one example set of compounds R₅ and R₆ are each independently C₁-C₄ alkyl.

In another example set of compounds R₅ and R₆ are each independently haloalkyl e.g., C₁-C₄ fluoroalkyl.

When R₃ and R₄ are taken together with C₂₀ to form C₃-C₆ cycloalkyl, an example is cyclopropyl.

In one embodiment, X₁ and X₂ are each H₂. In another embodiment, R₃ is hydrogen and R₄ is C₁-C₄ alkyl. In a preferred embodiment R₄ is methyl.

In another embodiment, R₅ and R₆ are each independently methyl, ethyl, fluoromethyl or trifluoromethyl. In a preferred embodiment, R₅ and R₆ are each methyl.

In yet another embodiment, R₁ and R₂ are each independently hydroxyl or OC(O)C₁-C₄ alkyl. In a preferred embodiment, R₁ and R₂ are each OC(O)C₁-C₄ alkyl in another preferred embodiment, R₁ and R₂ are each acetyloxy.

An example of such a compound is 1,3-O-diacetyl-1,25-dihydroxy-16-ene-24-keto-19-nor-cholecalciferol, having the following structure:

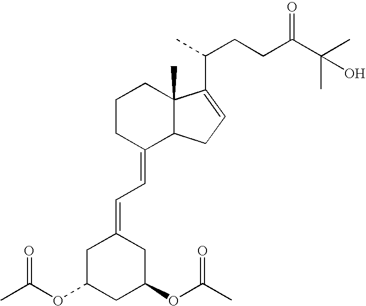

In another embodiment of the invention the vitamin D compound for use in accordance with the invention is 2-methylene-19-nor-20(S)-1-alpha-hydroxyvitamin D3:

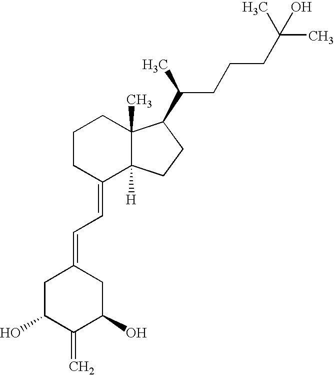

The synthesis of this compound is described in WO02/05823 and U.S. Pat. No. 5,536,713 which are herein incorporated in their entirety by reference.

In another embodiment of the invention, representing an embodiment of particular interest, the vitamin D compound for use in accordance with the invention is a compound of the formula I:

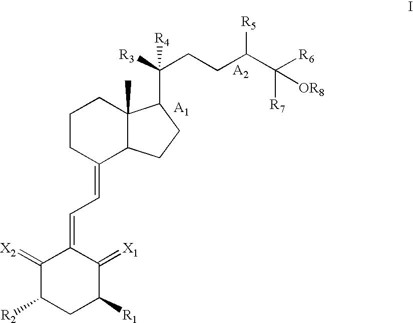

wherein:
A₁ is single or double bond;
A₂ is a single, double or triple bond;
X₁ and X₂ are each independently H₂ or CH₂, provided X₁ and X₂ are not both CH₂;
R₁ and R₂ are each independently OC(O)C₁-C₄ alkyl (including OAc), OC(O)hydroxyalkyl or OC(O)haloalkyl;
R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl;
R₆ and R₇ are each independently C₁₋₄alkyl or haloalkyl; and
R₈ is H, —COC₁-C₄alkyl (e.g. Ac), —COhydroxyalkyl or —COhaloalkyl; and
pharmaceutically acceptable esters, salts, and prodrugs thereof.

When R₃ and R₄ are taken together with C₂₀ to form C₃-C₆ cycloalkyl an example is cyclopropyl.

R₈ may typically represent H or Ac

In one embodiment, A₁ is a single bond and A₂ is a single bond, E or Z double bond, or a triple bond. In another embodiment, A₁ is a double bond and A₂ is a single bond, E or Z double bond, or a triple bond. One of ordinary skill in the art will readily appreciate that when A₂ is a triple bond, R₅ is absent.

In one embodiment, X₁ and X₂ are each H. In another embodiment, X₁ is CH₂ and X₂ is H₂.

In another embodiment, R₃ is hydrogen and R₄ is C₁-C₄ alkyl. In a preferred embodiment R₄ is methyl.

In another example set of compounds R₁ and R₂ both represent OAc.

In one set of example compounds R₆ and R₇ are each independently C₁₋₄alkyl. In another set of example compounds R₆ and R₇ are each independently haloalkyl. In another embodiment, R₆ and R₇ are each independently methyl, ethyl or fluoroalkyl. In a preferred embodiment, R₆ and R₇ are each trifluoroalkyl, e.g., trifluoromethyl.

Typically R₅ represents hydrogen.

Thus, in certain embodiments, vitamin D compounds for use in accordance with the invention are represented by I-a:

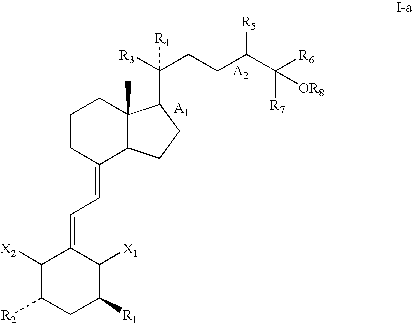

wherein:

A₁ is single or double bond;

A₂ is a single, double or triple bond;

X₁ and X₂ are each independently H or ═CH₂, provided X₁ and X₂ are not both ═CH₂;

R₁ and R₂ are each independently OC(O)C₁-C₄ alkyl, OC(O)hydroxyalkyl, or OC(O)haloalkyl;

R₃, R₄ and R₅ are each independently hydrogen, C₁-C₄ alkyl, hydroxyalkyl, or haloalkyl, or R₃ and R₄ taken together with C₂₀ form C₃-C₆ cycloalkyl;

R₆ and R₇ are each independently haloalkyl; and

R₈ is H, C(O)C₁-C₄ alkyl, C(O)hydroxyalkyl, or C(O)haloalkyl; and

pharmaceutically acceptable esters, salts, and prodrugs thereof.

An example compound of the above-described formula I-a is

### 1,3-di-O-acetyl-1,25-dihydroxy-16,23Z-diene-26,27-hexafluoro-19-nor-cholecalciferol (“Compound C”)

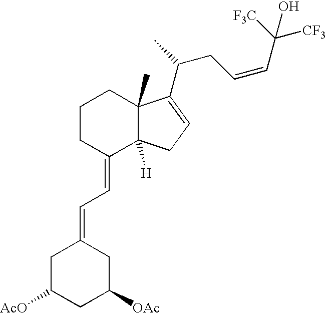

### “Compound C”

In another preferred embodiment, R₁ and R₂ are each OAc; A₁ is a double bond; A₂ is a triple bond; and R is either H or Ac for example the following compound:

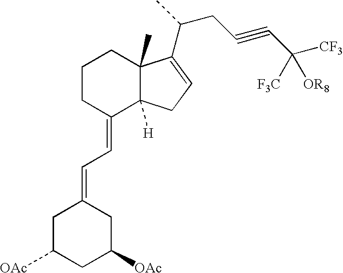

In certain embodiments of the above-represented formula I, vitamin D compounds for use in accordance with the invention are represented by the formula I-b:

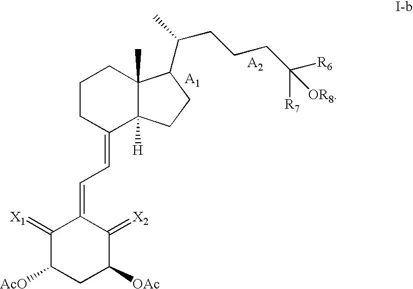

Other example compounds of the above-described formula I-b include:
1,3-di-O-acetyl-1,25-dihydroxy-23-yne-cholecalciferol;
1,3-di-O-acetyl-1,25-dihydroxy-16-ene-23-yne-cholecalciferol;
1,3-di-O-acetyl-1,25-dihydroxy-16,23E-diene-cholecalciferol;
1,3-di-O-acetyl-1,25-dihydroxy-16-ene-cholecalciferol;
1,3,25-Tri-O-acetyl-1,25-dihydroxy-16-ene-23-yne-26,27-hexafluoro-cholecalciferol:
1,3-di-O-acetyl-1,25-dihydroxy-16-ene-23-yne-26,27-hexafluoro-cholecalciferol;
1,3-Di-O-acetyl-1,25-dihydroxy-16,23E-diene-25R-26-trifluoro-cholecalciferol:
1,3-Di-O-acetyl-1,25-Dihydroxy-16-ene-23-yne-26,27-hexafluoro-19-nor-cholecalciferol:
1,3,25-Tri-O-acetyl-1,25-Dihydroxy-16-ene-23-yne-26,27-hexafluoro-19-nor-cholecalciferol:
1,3-di-O-acetyl-1,25-dihydroxy-16-ene-19-nor-cholecalciferol;
1,3-Di-O-acetyl-1,25-dihydroxy-16-ene-23-yne-19-nor-cholecalciferol:
1,3-Di-O-acetyl-1,25-dihydroxy-20-cyclopropyl-23-yne-19-nor-cholecalciferol:
1,3-Di-O-acetyl-1,25-dihydroxy-16-ene-23-yne-26,27-bishomo-19-nor-cholecalciferol;

In certain other embodiments of the above-represented formula I, the vitamin D compounds for use in accordance with the invention are represented by the formula I-c:

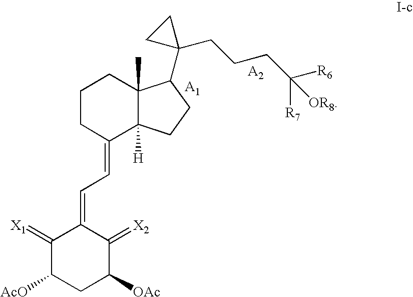

Other example compounds of the above-described formula I-b include:
1,3,25-tri-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-26,27-hexafluoro-19-nor-cholecalciferol;
1,3-di-O-acetyl-1,25-dihydroxy-20-cyclopropyl-23-yne-cholecalciferol;
1,3-di-O-acetyl-1,25-dihydroxy-20-cyclopropyl-23E-ene-26,27-hexafluoro-19-nor-cholecalciferol;
1,3-di-O-acetyl-1,25-dihydroxy-20-cyclopropyl-23Z-ene-26,27-hexafluoro-19-nor-cholecalciferol;
1,3-di-O-acetyl-1,25-dihydroxy-20-cyclopropyl-cholecalciferol;
1,3-di-O-acetyl-1,25-dihydroxy-16-ene-20-cyclopropyl-19-nor-cholecalciferol; and

1,3-Dd-O-acetyl-1,25-dihydroxy-16-ene-20-cyclopropyl-cholecalciferol;

In another preferred embodiment, vitamin D compounds for use in accordance with the invention are compounds of the formula:

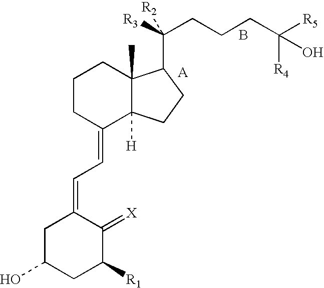

wherein

### X is H₂ or CH₂;

R₁ is hydrogen, hydroxy or fluorine;
R₂ is hydrogen or methyl;
R₃ is hydrogen or methyl, when R₂ or R₃ is methyl, R₃ or R₂ must be hydrogen;
R₄ is methyl, ethyl or trifluoromethyl;
R₅ is methyl, ethyl or trifluoromethyl;
A is a single or double bond; and
B is a single, E-double, Z-double or triple bond.

In particularly preferred compounds, each of R₄ and R₅ is methyl or ethyl, for example 1-alpha-fluoro-25-hydroxy-16,23E-diene-26,27-bishomo-20-epi-cholecalciferol (Compound A in the following examples), having the formula:

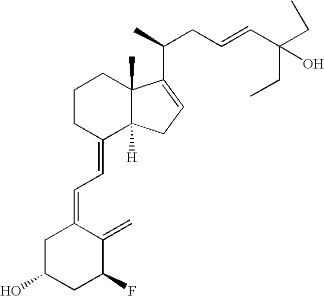

Such compounds are described in U.S. Pat. No. 5,939,408 and EP808833, the contents of which are herein incorporated by reference in their entirety. The invention also embraces use of esters and salts of Compound A. Esters include pharmaceutically acceptable labile esters that may be hydrolysed in the body to release Compound A. Salts of Compound A include adducts and complexes that may be formed with alkali and alkaline earth metal ions and metal ion salts such as sodium, potassium and calcium ions and salts thereof such as calcium chloride, calcium malonate and the like. However, although Compound A may be administered as a pharmaceutically acceptable salt or ester thereof, preferably Compound A is employed as is i.e., it is not employed as an ester or a salt thereof. Other preferred vitamin D compounds for use in accordance with the invention included those having formula I-a:

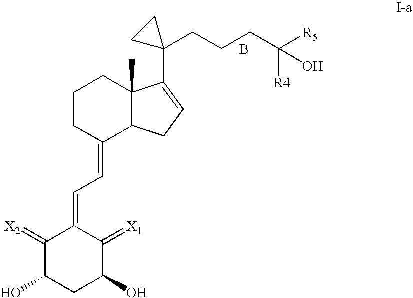

wherein:

B is single, double, or triple bond;

X₁ and X₂ are each independently H₂ or CH₂, provided X₁ and X₂ are not both CH₂; and

R₄ and R₅ are each independently alkyl or haloalkyl.

Compounds of formula I-a including the following:

### 1,25-Dihydroxy-16-ene-23-yne-20-cyclopyl-cholecalciferol

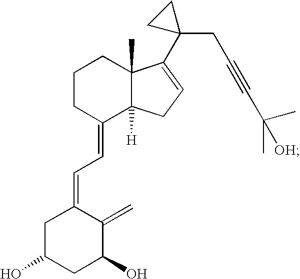

### 1,25-Dihydroxy-16-ene-23-yne-20-cyclopropyl-19-nor-cholecalciferol

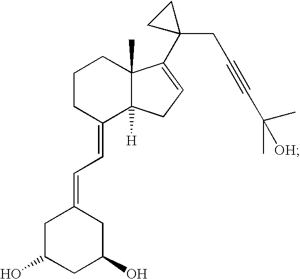

### 1,25-Dihydroxy-16-ene-20-cyclopropyl-23-yne-26,27-hexafluoro-19-nor-cholecalciferol

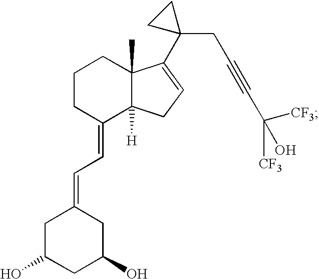

### 1,25-Dihydroxy-16-ene-20-cyclopropyl-23-yne-26,27-hexafluoro-cholecalciferol

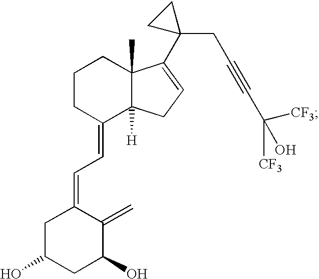

### 1,25-Dihydroxy-16,23E-diene-20-cyclopropyl-26,27-hexafluoro-19-nor-cholecalciferol

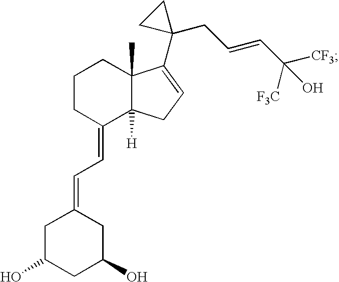

### 1,25-Dihydroxy-16,23E-diene-20-cyclopropyl-26,27-hexafluoro-cholecalciferol

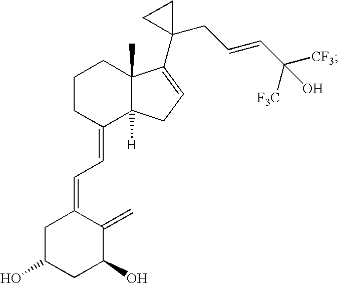

### 1,25-Dihydroxy-16,23Z-diene-20-cyclopropyl-26,27-hexafluoro-19-nor-cholecalciferol

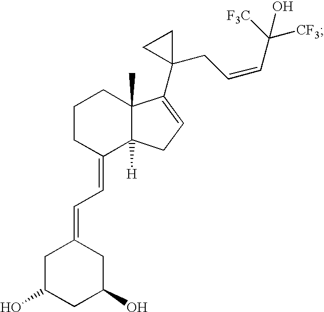

### 1,25-Dihydroxy-16,23Z-diene-20-cyclopropyl-26,27-hexafluoro-cholecalciferol

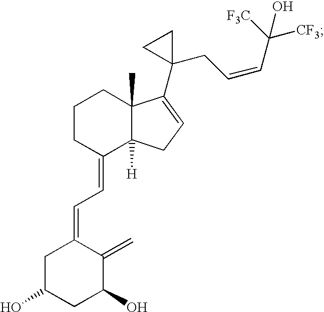

### 1,25-Dihydroxy-16-ene-20-cyclopropyl-19-nor-cholecalciferol

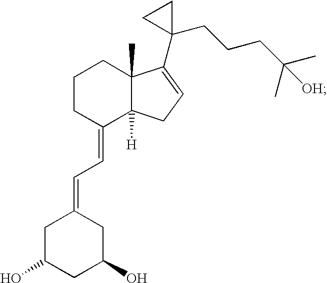

and

### 1,25-Dihydroxy-16-ene-20-cyclopropyl-cholecalciferol

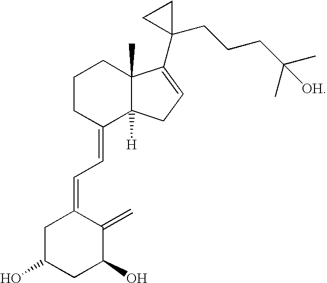

Another vitamin D compounds of the invention is 1,25-dihydroxy-21(3-hydroxy-3-trifluoromethyl-4-trifluoro-butynyl)-26,27-hexadeutero-19-nor-20S-cholecalciferol.

The use of compounds having the structures given above is extended to pharmaceutically acceptable esters, salts, and prodrugs thereof. Examples are given in the previous paragraph.

A vitamin D compound of particular interest is calcitriol.

Other example compounds of use in the invention which are vitamin D receptor agonists include paricalcitol (ZEMPLAR™) (see U.S. Pat. No. 5,587,497), tacalcitol BONALFA™) (see U.S. Pat. No. 4,022,891), doxercalciferol (HECTOROL™) (see Lam et al. (1974) Science 186, 1038), maxacalcitol (OXAROL™) (see U.S. Pat. No. 4,891,364), calcipotriol (DAIVONEX™) (see U.S. Pat. No. 4,866,048), and falecalcitriol (FULSTAN™).

Other compounds include ecalcidene, calcithiazol and tisocalcitate. Other compounds include atocalcitol, lexacalcitol and seocalcitol. Another compound of possible interest is secalciferol (“OSTEO D”). Other non-limiting examples of vitamin D compounds that may be of use in accordance with the invention include those described in published international applications: WO 01/40177, WO0010548, WO0061776, WO0064869, WO0064870, WO00066548, WO0104089, WO0116099, WO0130751, WO0140177, WO0151464, WO0156982, WO0162723, WO0174765, WO0174766, WO0179166, WO0190061, WO0192221, WO0196293, WO02066424, WO02121.82, WO0214268, WO03004036, WO03027065, WO03055854, WO03088977, WO04037781, WO04067504, WO8000339, WO8500819, WO8505622, WO08602078, WO8604333, WO8700834, WO8910351, WO9009991, WO9009992, WO9010620, WO9100271, WO9100855, WO9109841, WO9112239, WO9112240, WO9115475, WO9203414, WO9309093, WO9319044, WO9401398, WO9407851, WO9407852, WO9408958, WO9410139, WO9414766, WO9502577, WO9503273, WO9512575, WO9527697, WO9616035, WO9616036, WO9622973, WO9711053, WO9720811, WO9737972, WO9746522, WO9818759, WO9824762, WO9828266, WO9841500, WO9841501, WO9849138, WO9851663, WO9851664, WO9851678, WO9903829, WO9912894, WO9915499, WO9918070, WO9943645, WO9952863, those described in U.S. Pat. No. 3,856,780, U.S. Pat. No. 3,994,878, U.S. Pat. No. 4,021,423, U.S. Pat. No. 4,026,882, U.S. Pat. No. 4,028,349, U.S. Pat. No. 4,225,525, U.S. Pat. No. 4,613,594, U.S. Pat. No. 4,804,502, U.S. Pat. No. 4,898,855, U.S. Pat. No. 5,039,671, U.S. Pat. No. 5,087,619, U.S. Pat. No. 5,145,846, U.S. Pat. No. 5,247,123, U.S. Pat. No. 5,342,833, U.S. Pat. No. 5,428,029, U.S. Pat. No. 5,451,574, U.S. Pat. No. 5,612,328, U.S. Pat. No. 5,747,479, U.S. Pat. No. 5,804,574, U.S. Pat. No. 5,811,414, U.S. Pat. No. 5,856,317, U.S. Pat. No. 5,872,113, U.S. Pat. No. 5,888,994, U.S. Pat. No. 5,939,408, U.S. Pat. No. 5,962,707, U.S. Pat. No. 5,981,780, U.S. Pat. No. 6,017,908, U.S. Pat. No. 6,030,962, U.S. Pat. No. 6,040,461, U.S. Pat. No. 6,100,294, U.S. Pat. No. 6,121,312, U.S. Pat. No. 6,329,538, U.S. Pat. No. 6,331,642, U.S. Pat. No. 6,392,071, U.S. Pat. No. 6,452,028, U.S. Pat. No. 6,479,538, U.S. Pat. No. 6,492,353, U.S. Pat. No. 6,537,981, U.S. Pat. No. 6,544,969, U.S. Pat. No. 6,559,138, U.S. Pat. No. 6,667,298, U.S. Pat. No. 6,683,219, U.S. Pat. No. 6,696,431, U.S. Pat. No. 6,774,251, and those described in published US Patent Applications: US2001007907, US2003083319, US2003125309, US2003130241, US2003171605, US2004167105.

It will be noted that the structures of some of the compounds of the invention include asymmetric carbon atoms. Accordingly, it is to be understood that the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

The preferred stereochemistry of compounds is as represented absolutely by the structures disclosed herein.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Eniantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

Synthetic methods for preparation of anti-proliferative agents will generally be known from published sources. For example, processes for preparing epirubicin are disclosed in U.S. Pat. No. 5,945,518.

Typically a single anti-proliferative agent is employed in combination with the vitamin D compound.

A preferred combination of agents for use in the invention are (i) calcitriol and doxorubicin; and (ii) calcitriol and epirubicin. As demonstrated by the examples, these compounds show a favourable synergistic interaction in a model for bladder cancer. The synergistic interaction between calcitriol and epirubicin appears to be particularly favourable.

The invention also provides a pharmaceutical composition, comprising an effective amount of a vitamin D compound as described herein and one or more anti-proliferative agents and a pharmaceutically acceptable carrier. In a further embodiment, the effective amount is effective to treat bladder cancer, as described previously.

In an embodiment, the vitamin D compound and/or the one or more anti-proliferative agents are administered to the subject using a pharmaceutically-acceptable formulation, e.g., a pharmaceutically-acceptable formulation that provides sustained delivery of the vitamin D compound and/or the one or more anti-proliferative agents to a subject for at least: 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intrarectally, for example, as a suppository, cream or foam; or (5) aerosol, for example, as an aqueous aerosol, liposomal preparation or solid particles containing the compound.

In a preferred embodiment the vitamin D compound is administered via topical bladder instillation, in other words intravesical administration, thus avoiding or reducing the likelihood of hypercalcemia, and is administered in a total dose within the bladder at each instillation of about 0.01 mg, 0.05 mg, 0.1 mg, 0.5 mg, 1 mg, 5 mg, 10 mg, 20 mg or 50 mg.

Vitamin D compounds of the invention may thus be formulated in alcoholic solutions, which when diluted in, for example, 10 to 100 ml 5-30% ethanol solution and instilled in the bladder have a final concentration of for example at about 10 μM, 50 μM, 100 μM, 1 mM, 2 mM, 5 mM, or 10 mM.

In a preferred embodiment the other anti-proliferative agents, e.g. an anthracycline such as epirubicin or doxorubicin, are also administered via topical bladder instillation, in other words intravesical administration, in order to avoid or reduce the likelihood of side effects such as systemic toxicity, and are typically administered in a total dose within the bladder at each instillation of about 0.1 to about 500 mg, for example 20-150 mg (such as 40-100 mg).

Should the choice of anti-proliferative compound make it preferable that alcohol should be avoided in any formulation of the vitamin D compound to be co-administered or sequentially administered with it, said vitamin D compound can be formulated with a stabilising agent, for example formulated as a complex with beta-cyclodextrin, thus avoiding the use of ethanol or formulated with a nonionic solubilizer and emulsifier such as Cremophor™EL (polyoxyethyleneglycol triricinoleate; BASF Aktiengesellschaft). Alternatively, the vitamin D compound may be formulated in aqueous solutions as described, for example, in U.S. Pat. No. 4,308,264, U.S. Pat. No. 6,051,567, U.S. Pat. No. 6,265,392, U.S. Pat. No. 6,274,169, WO 96/36340.

Anti-proliferative compounds of the invention, in particular an anthracycline compound may also be formulated for ‘intravesical’ instillation as known in the art for the introduction of agents which are placed inside the bladder. Such formulations can be administered sequentially with a vitamin D compound formulation or alternatively the formulation can contain both a vitamin D compound and one or more other anti-proliferative compound for co-administration together. The anti-proliferative agent can be administered intravesically, for example, as 30-100 mg doxomubicin at a concentration of 1 mg/mL or 800 mg valrubicin (in a stock solution of 20 mL) diluted in 55 mL of saline. Epirubicin for intravesical instillations in humans is typically used at 2 mg/ml, generally instilled in a volume of 25-50 ml. Instillation schedules vary from weekly to every 3 weeks for up to and beyond 6 weeks. Regular maintenance therapy is common. Following intravesical administration, these agents should be retained in the bladder for 1-2 hours.

A preferred pharmaceutical composition according to the invention comprises (e.g. as a solution) a vitamin D compound, one or more other anti-proliferative agents and one or more pharmaceutically acceptable carriers suitable for administration to the bladder by intravesical instillation. Example carriers include water, optionally water/ethanol mixtures.

Our experiments have shown compatibility between epirubicin and calcitriol in a solution suitable for instillation.

It is important that whatever formulation is chosen for instillation achieves a homogenous distribution of the active substances on the target tissue of the bladder, and is formulated at an optimum and maximum instillation volume, at a concentration of ethanol (if used) tolerated by the bladder mucosa (as known in the art) and in an acceptable pH range. Those skilled in the art will be aware of appropriate formulation for intravesical instillation that are compatible with the chosen vitamin D compound and anti-proliferative compound.

Vitamin D compounds and anti-proliferative compounds of the invention may be formulated in heterogenous systems as known as the air, for example, solid lipid nanospheres, liposomes, micelles, microemulsions, macroemulsions, macro-micro emulsions, and similar carriers (see, e.g., D. O. Shah (ed), 1998, Micelles, Microemulsions, and Monolayers: Science and Technology, Marcel Dekker; A. S. Janoff (ed), 1998, Liposomes: Rational Design, Marcel Dekker).

In one embodiment, vitamin D compounds and anti-proliferative compounds of the invention are formulated in liposomes for topical bladder instillation, Liposomes can be produced in accordance with established methods (see, e.g., G. Cregoriadis (ed.), 1993, Liposome Technology Vols. 1-3, CRC Press, Boca Raton, Fla., or Szoka and Papahadjopoulos, 1978, Proc. Natl. Acad. Sci. USA 75:4194-4198). Vitamin D compounds of the invention, for example calcitriol, may be formulated in liposomal preparations (see, e.g. Körbela, et al. (2001) Skin Pharmacology and Applied Skin Physiology 14:291-295) Anti-proliferative compounds of the invention, for example, Adriamycin, daunomycin, and epirubicin may be encapsulated into a liposome by means of a remote loading method that takes advantage of a pH gradient (see D. M. Lawrence et al., 1989, Cancer Research 49:5922).

In an alternative embodiment, vitamin D compounds and anti-proliferative compounds of the invention may be formulated in a floating insert placed within the bladder, for example comprising polymers with different molecular weights which regulate the releasing rate of one or more of the components therein. Bio-erodible, sustained release preparations for placement into the bladder through the urethra which are useful in the present invention to provide sustained release of drugs are described in, for example, U.S. Pat. No. 6,207,180. Such devices is bio-eroded during or after the sustained release of the active ingredients (for example a vitamin D compound and/or an anti-proliferative compound of the invention) such that there is no blockage of the urinary tract while the device is in place within the bladder.

Anti-proliferative agents of the invention may be formulated or conjugated as known in the art to target specific cancer cells or improve its efficacy, for example Plasmin-targeted Doxorubicin, Liposome-encapsulated Doxorubicin (LED), Transdrug Doxorubicin (a doxorubicin-PolyIsoHexilCyanoAcrylate conjugate) or anthracycline-formaldehyde conjugates for example Doxoform, Daunoform, and Epidoxoform described by Taatjes D J et al. Chem Res Toxicol. 1999 12(7):588-96.

Vitamin D compounds and anti-proliferative compounds of the invention may also be administered directly to the bladder wall by iontophoretic delivery by means of a balloon catheter such as that described in U.S. Pat. No. 5,749,845.

In accordance with the practice of the invention, the composition may be in a solid state or in solution. Preferably, when in solution, the composition has a pH of between 7 and 12.

The therapeutic composition may also contain an osmolar component that provides an isotonic or nearly isotonic solution compatible with human cells and blood. Typically the osmolar component is a salt, such as sodium chloride, or a sugar or a combination of two or more of these components. The sugar may be a monosaccharide such as dextrose, a disaccharide such as sucrose or lactose, a polysaccharide such as dextran 40, dextran 60, 10 or starch, or a sugar alcohol such as mannitol. It should be obvious to those skilled in the art that all components of the composition contribute to the osmolarity of the solution but to achieve an isotonic or near-isotonic solution, the contributions of these components should be taken into account to ensure that the proper osmolar component is added and not added in excess which would result in a hypertonic solution.

Due to the permeable nature of the urothelium, a preferred solution would be isotonic or near isotonic. Hypotonic solutions are known to result in cell lysis, particularly of red blood cells, but other cells may also be damaged leading to increased cell damage in the bladder and accessible underlying layers. Hypertonic solutions may result in cell shrinkage which may enlarge pores or weaken cell junctions allowing urinary solutes more access to underlying cell layers leading to further damage, pain and inflammation. The addition of an osmolar component to the composition to form an isotonic or near isotonic solution ensures that neither of these two possibilities occur. Typically, the osmolar component is 0.9% sodium chloride, or somewhat less as the other components in the solution also contribute to the solution's osmolarity and thus should be taken into account. Typically the osmolar component is a salt, such as sodium chloride, or a sugar or a combination of two or more of these components. The sugar may be a monosaccharide such as dextrose, a disaccharide such as sucrose or lactose, a polysaccharide such as dextran 40, dextran 60, or starch, or a sugar alcohol such as mannitol. It should be obvious to those skilled in the art that all components of the composition contribute to the osmolarity of the solution but to achieve an isotonic or near-isotonic solution, the contributions of these components should be taken into account to ensure that the proper osmolar component is added and not added in excess which would result in a hypertonic solution. The osmolar component of the compositions of the invention includes but is not limited to sodium chloride, dextrose, dextran 40, dextran 60, starch and mannitol, or a combination thereof. The amount of the osmolar component in the compositions of the invention will vary 10 depending on the subject, severity and course of the disease, the subject's health and response to treatment and the judgment of the treating physician. Accordingly, the dosages of the molecules should be titrated to the individual subject. For example, the amount of the osmolar component(s) in the compositions of the invention is at least 50 milliosmoles.

Solutions for instillation may optionally include formulation components intended to enhance adhesion to or persistence at the bladder wall. Example formulation components include bioadhesive agents such as polyvinylpyrrolidone, hydroxypropylmethylcellulose, carbomer, sodium alginate, pectin and the like. Further example formulation components include a gelling agent, for example a thermosensitive gelling agent. An example thermosensitive gelling agent is described in P. Tyagi et al., “Sustained Intravesical Drug Delivery Using Thermosensitive Hydrogel,” Pharm. Res. 21:832-837 (2004). In general, these thermosensitive gelling agents are solutions at room temperature but develop increased viscosity when subjected to raised temperature (for example through contact with body tissue, such as the bladder wall). Other gelling agents are known in the art. C. Le Visage et al. “Efficacy of paclitaxel released from bio-adhesive polymer microspheres on model superficial bladder cancer” J. Urol. 2004 171(3) 1324-1329 discloses the use of anticancer agents in polymer microspheres. Such microspheres may also be of use in the present invention.

The phrase “pharmaceutically acceptable” refers to those vitamin D compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D compound(s) and/or other anti-proliferative agents include those suitable for intravesical, oral, nasal, topical (including buccal and sublingual), rectal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect. Generally, out of one hundred percent, this amount will range from about 0.1 to about 99.5 percent e.g. from about 1 percent to about 99 percent of active ingredient or else from about 0.5 percent to about 90 percent, preferably from about 5 percent to about 70 percent, most preferably from about 10 percent to about 30 percent by weight.

Methods of preparing these compositions include the step of bringing into association a vitamin D compound(s) and/or the other anti-proliferative agents with the carrier and, optionally, one or more accessory ingredients. In general, the formulations are prepared by uniformly and intimately bringing into association a vitamin D compound and/or one more other anti-proliferative agents with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D compound(s) and/or one or more anti-proliferative agents as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents in the case of capsules, tablets and pills, the pharmaceutical compositions may also comprise buffering agents. Solid compositions of a similar type may also be employed as fillers in soft and hard-failed gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that releases the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for intravesical or oral administration of the vitamin D compound(s) and/or one or more anti-proliferative agents include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the intravesical or oral compositions can include adjuvants such as wetting agents, emulsifying and suspending agents, sweetening, flavouring, coloring, perfuming and preservative agents.

Suspensions, in addition to the active vitamin D compound(s) and/or one or more anti-proliferative agents may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal administration may be presented as a suppository, which may be prepared by mixing one or more vitamin D compound(s) and/or one or more anti-proliferative agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum and release the active agent.

Dosage forms for the topical or transdermal administration of a vitamin D compound(s) and/or one or more anti-proliferative agents include powders, sprays, ointments, pastes, creams, lotions, gels, solutions, patches and inhalants. The active vitamin D compound(s) and/or one or more anti-proliferative agents may be mixed under sterile conditions with a pharmaceutically-acceptable carrier, and with any preservatives, buffers, or propellants which may be required.

The ointments, pastes, creams and gels may contain, in addition to vitamin D compound(s) and/or one or more anti-proliferative agents of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D compound(s) and/or one or more anti-proliferative agents, excipients such as lactose, talc, silicic acid, aluminium hydroxide, calcium silicates and polyamide powder, or mixtures of these substances. Sprays can additionally contain customary propellants, such as chlorofluorohydrocarbons or hydrofluoroalkanes such as HFA134a or HFA227 and volatile unsubstituted hydrocarbons, such as butane and propane.

The vitamin D compound(s) and/or one or more anti-proliferative agents can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D compound(s) and/or one or more anti-proliferative agents to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral administration comprise one or more vitamin D compound(s) and/or one or more anti-proliferative agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D compound(s) and/or one or more anti-proliferative agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Regardless of the route of administration selected, the vitamin D compound(s) and/or one or more anti-proliferative agents, which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 300 ug per day. An exemplary dose range of Compound A is from 0.1 to 300 ug per day, for example 50-150 ug per day e.g., 75 or 150 ug per day. A unit dose formulation preferably contains 50-150 ug e.g., 75 or 150 ug and is preferably administered once per day.

A preferred dose of the vitamin D compound for the present invention is the maximum that a patient can tolerate and not develop hypercalcemia. Preferably, the vitamin D compound of the present invention is administered at a concentration of about 0.001 ug to about 100 ug per kilogram of body weight, about 0.001 to about 10 ug/kg or about 0.001 ug to about 100 ug/kg of body weight. Ranges intermediate to the above-recited values are also intended to be part of the invention. Where the vitamin D compound is administered intravesically, the dose may be increased to the optimal effective dose, for example, at a total dose within the bladder at each instillation of about 0.01 mg, 0.05 mg, 0.1 mg, 0.5 mg, 1 mg, 5 mg, 10 mg, 20 mg or 50 mg, preferably without a corresponding increase in serum calcium.

The invention also includes a packaged formulation including a pharmaceutical composition comprising a vitamin D compound, a pharmaceutical composition comprising one or more other anti-proliferative agents (the one or more anti-proliferative agents optionally being within the same pharmaceutical composition as the vitamin D compound) and a pharmaceutically acceptable carrier packaged with instructions for use in the prevention and/or treatment of bladder cancer according to the invention.

For assistance in formulating the compositions of the present invention, one may refer to Remington's Pharmaceutical Sciences, 15th Edition, Mack Publishing Co., Easton, Pa.

### II. Synthesis of Compounds

A number of the compounds for use in the present invention can be prepared by incubation of vitamin D₃ analogues in cells, for example, incubation of vitamin D₃ analogues in either UMR 106 cells or Ros 17/2.8 cells results in production of vitamin D₃ compounds for use in the invention. For example, incubation of 1,25-dihydroxy-16-ene-5,6-trans-calcitriol in UMR 106 cells results in production of 1,25-dihydroxy-16-ene-24-oxo-5,6-trans-calcitriol.

In addition to the methods described herein, compounds of the present invention can be prepared using a variety of synthetic methods. For example, one skilled in the art would be able to use methods for synthesizing existing vitamin D₃ compounds to prepare compounds of use in the invention (see e.g., Bouillon, R. et al. (1995) Endocr. Rev. 16(2):200-257; Ikekawa, N. (1987) Med. Res. Rev. 7:333-366; DeLuca, H. F. and Ostrem, V. K. (1988) Prog Clin. Biol. Res. 259:41-55; Ikekawa, N. and Ishizuka, S. (1992) CRC Press 8:293-316; Calverley, M. J. and Jones, G. (1992) Academic Press 193-270; Pardo, R. and Santelli, M. (1985) Bull. Soc. Chim. Fr:98-114; Bythgoe, B. (1980) Chem. Soc. Rev. 449-475; Quinkert, G. (1985) Synform 3:41-122; Quinkert, G. (1986) Synform 4:131-256; Quinkert, G. (1987) Synform 5:1-85; Mathieu, C. et al. (1994) Diabetologia 37:552-558; Dai, H. and Posner, G. H. (1994) Synthesis 1383-1398; and DeLuca, et al., WO 97/11053).

Exemplary methods of synthesis include the photochemical ring opening of a 1-hydroxylated side chain-modified derivative of 7-dehydrocholesterol which initially produces a previtamin that is easily thermolyzed to vitamin D₃ in a well known fashion (Barton, D. H. R. et al. (1973) J. Am. Chem. Soc. 95:2748-2749; Barton, D. H. R. (1974) JCS Chem. Comm. 203-204); phosphine oxide coupling method developed by (Lythgoe, et al. (1978) JCS Perkin Trans. 1:590-595) which comprises coupling a phosphine oxide to a Grundmann's ketone derivative to directly produce a 1-alpha,25(OH)₂D₃ skeleton as described in Baggiolini, E. G., et al. (1986) J. Org. Chem. 51:3098-3108; DeSchrijver, J. and DeClercq, P. J. (1993) Tetrahed Lett 34:4369-4372; Posner, G. H and Kinter, C. M. (1990) J. Org. Chem. 55:3967-3969; semihydrogenation of dienynes to a previtamin structure that undergoes rearrangement to the corresponding vitamin D₃ analogue as described by Harrison, R. G. et al. (1974) JCS Perkin Trans. 1:2654-2657; Castedo, L. et al. (1988) Tetrahed Lett 29:1203-1206; Mascarenas, J. S. (1991) Tetrahedron 47:3485-3498; Barrack, S. A. et al. (1988) J. Org. Chem. 53:1790-1796) and Okamura, W. H. et al. (1989) J. Org. Chem. 54:4072-4083; the vinylallene approach involving intermediates that are subsequently arranged using heat or a combination of metal catalyzed isomerization followed by sensitized photoisomerization. (Okamura, W. H. et al. (1989) J. Org. Chem. 54:4072-4083; Van Alstne, E. M. et al. (1994) J. Am. Chem. Soc. 116:6207-6210); the method described by Trost, B. M. et al. J. Am. Chem. Soc. 114:9836-9845; Nagasawa, K. et al. (1991) Tetrahed Lett 32:4937-4940 involves an acyclic A-ring precursor which is intramolecular cross-coupled to the bromoenyne leading directly to the formation of 1,25(OH)₂D₃ skeleton; a tosylated derivative which is isomerized to the i-steroid that can be modified at carbon-1 and then subsequently back-isomerized under sovolytic conditions to form 1-alpha,25(OH)₂D₂ or analogues thereof (Sheves, M. and Mazur, Y. (1974) J. Am. Chem. Soc. 97:6249-6250; Paaren, H. E. et al. (1980) J. Org. Chem. 45:3253-3258; Kabat, M. et al. (1991) Tetrahed Lett 32:2343-2346; Wilson, S. R. et al. (1991) Tetrahed Lett 32:2339-2342); the direct modification of vitamin D derivatives to 1-oxygenated 5,6-trans vitamin. D as described in (Andrews, D. R. et al. (1986) J. Org. Chem. 51:1635-1637); the Diels-Alders cycloadduct method of previtamin D₃ can be used to cyclorevert to 1-alpha,25(OH)₂D₂ through the intermediary of a previtamin form via thermal isomerization (Vanmaele, L. et al. (1985) Tetrahedron 41:141-144); and, a final method entails the direct modification of 1-alpha,25(OH)₂D₂ or an analogue through use of suitable protecting groups such as transition metal derivatives or by other chemical transformations (Okarmura, W. H. et al. (1992) J. Cell Biochem. 49:10-18). Additional methods for synthesizing vitamin D₂ compounds are described in, for example, Japanese Patent Disclosures Nos. 62750/73, 26858/76, 26859/76, and 71456/77; U.S. Pat. Nos. 3,639,596; 3,715,374; 3,847,955 and 3,739,001.

Examples of the compounds of use in this invention having a saturated side chain can be prepared according to the general process illustrated and described in U.S. Pat. No. 4,927,815. Examples of compounds of the invention having an unsaturated side chain can be prepared according to the general process illustrated and described in U.S. Pat. No. 4,847,012. Examples of compounds of the invention wherein R groups at position C20 together represent a cycloalkyl group can be prepared according to the general process illustrated and described in U.S. Pat. No. 4,851,401.

Another synthetic strategy for the preparation of side-chain-modified analogues of 1-alpha,25-dihydroxyergocalciferol is disclosed in Kutner et al., The Journal of Organic Chemistry, 1988, 53:3450-3457. In addition, the preparation of 24-homo and 26-homo vitamin D analogues are disclosed in U.S. Pat. No. 4,717,721.

The enantioselective synthesis of chiral molecules is now state of the art. Through combinations of enantioselective synthesis and purification techniques, many chiral molecules can be synthesized as an enantiomerically enriched preparation. For example, methods have been reported for the enantioselective synthesis of A-ring diastereomers of 1-alpha,25(OH)₂D₃ as described in Muralidharan et al. (1993) J. Organic Chem. 58(7): 1895-1899 and Norman et al. (1993) J. Biol. Chem. 268(27): 20022-30. Other methods for the enantiomeric synthesis of various compounds known in the art include, inter alia, epoxides (see, e.g., Johnson, R. A.; Sharpless, K. B. in Catalytic Asymmetric Synthesis; Ojima, I., Ed.: VCH: New York, 1993; Chapter 4.1. Jacobsen, E. N. ibid. Chapter 4.2), diols (e.g., by the method of Sharpless, J. Org. Chem. (1992) 57:2768), and alcohols (e.g., by reduction of ketones, E. J. Corey et al., J. Am. Chem. Soc. (1987) 109:5551). Other reactions useful for generating optically enriched products include hydrogenation of olefins (e.g., M. Kitamura et al., J. Org. Chem. (1988) 53:708); Diels-Alder reactions (e.g., K. Narasaka et al., J. Am. Chem. Soc. (1989) 111:5340); aldol reactions and alkylation of enolates (see, e.g., D. A. Evans et al., J. Am. Chem. Soc. (1981) 103:2127; D. A. Evans et al., J. Am. Chem. Soc. (1982) 104:1737); carbonyl additions (e.g., R. Noyori, Angew. Chem. Int. Ed. Eng. (1991) 30:49); and ring-opening of meso-epoxides (e.g., Martinez, L. E.; Leighton J. L., Carsten, D. H.; Jacobsen, E. N. J. Am. Chem. Soc. (1995) 117:5897-5898). The use of enzymes to produce optically enriched products is also well known in the art (e.g., M. P. Scheider, ed. “Enzymes as Catalysts in Organic Synthesis”, D. Reidel, Dordrecht (1986).

Chiral synthesis can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

### III. Examples of Chemical Synthesis of Certain Preferred Compounds

### Experimental

Further details of the synthesis of the following compounds are described in WO2005/030223.

All operations involving vitamin D₃ analogues were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 um mesh silica gel. Preparative HPLC was performed on a 5×50 cm column and 15-30 um mesh silica gel at a flow rate of 100 ml/min.

### Example 1

### Synthesis of 1,3-Di-O-acetyl-1,25-dihydroxy-16,23Z-diene-26,27-hexafluoro-19-nor-cholecalciferol (1)

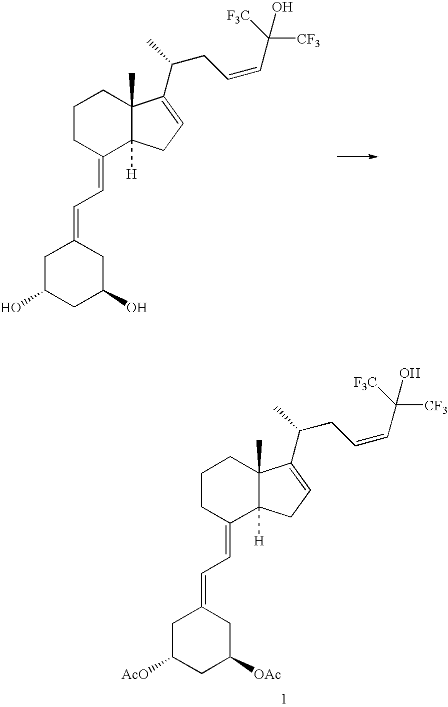

The starting material 1,25-dihydroxy-16,23Z-diene-26,27-hexafluoro-19-nor-cholecalciferol can be prepared as described in U.S. Pat. No. 5,428,029 to Doran et al. 3 mg of 1,25-dihydroxy-16,23Z-diene-26,27-hexafluoro-19-nor-cholecalciferol was dissolved in 0.8 ml of pyridine, cooled to ice-bath temperature and 0.2 ml of acetic anhydride was added and maintained at that temperature for 16 h. Then the reaction mixture was diluted with 1 ml of water, stirred for 10 min in the ice bath and distributed between 5 ml of water and 20 ml of ethyl acetate. The organic layer was washed with 3×5 ml of water, once with 5 ml of saturated sodium hydrogen carbonate, once with 3 ml of brine then dried (sodium sulfate) and evaporated. The oily residue was taken up in 1:6 ethyl acetate-hexane and flash-chromatographed using a stepwise gradient of 1:6, 1:4 and 1:2 ethyl acetate-hexane. The column chromatography was monitored by TLC (1:4 ethyl acetate-hexane, spot visualization with phosphomolybdic acid spray), the appropriate fractions were pooled, evaporated, the residue taken up in methyl formate, filtered, then evaporated again to give 23.8 mg of the title compound (1) as a colorless syrup; 400 MHz ¹H NMR δ 0.66 (3H., s), 0.90 (1H, m), 1.06 (3H, d, J=7.2 Hz), 1.51 (1H, m), 1.72-1.82 (3H, m), 1.9-2.1 (3H, m), 1.99 (3H, s) 2.04 (3H, s), 2.2-2.3 (3 m), 2.44-2.64 (6H, m), 2.78 (1H, m), 3.01 (1H, s), 5.10 (2H, m). 5.38 (1H, m), 5.43 (1H, d, J=12 Hz), 5.85 (1H, d, J=1:1.5 Hz), 5.97 (1H, dt, J=12 and 7.3 Hz), 6.25 (1H, d, J=11.5 Hz).

### Example 2

### Synthesis of 1,3-Di-O-acetyl-1,25-Dihydroxy-16-ene-23-yne-26,27-hexafluoro-19-nor-cholecalciferol (2) and 1,3,25-Tri-O-acetyl-1,25-Dihydroxy-16-ene-23-yne-26,27-hexafluoro-19-nor-cholecalciferol (3)

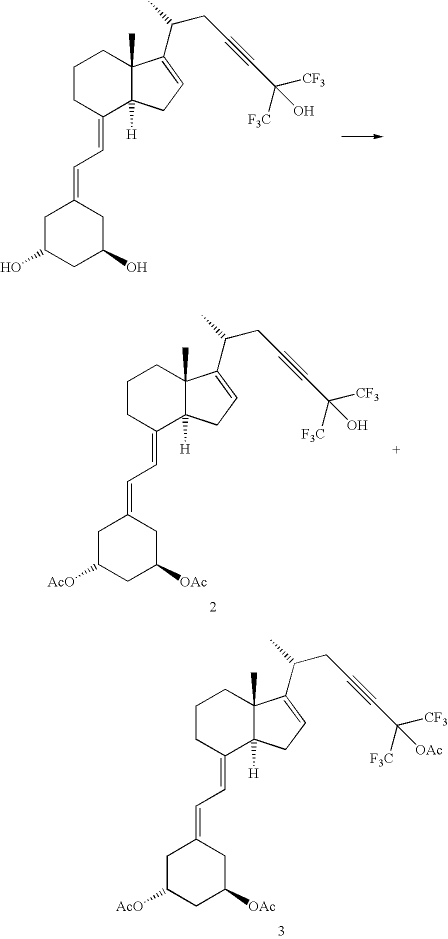

The starting material 1,25-dihydroxy-16-ene-23-yne-26,27-hexafluoro-19-nor-cholecalciferol can be prepared as described in U.S. Pat. Nos. 5,451,574 and 5,612,328 to Baggiolini et al. 314 mg (0.619 mmole) of 1,25-dihydroxy-16-ene-23-yne-26,27-hexafluoro-19-nor-cholecalciferol was dissolved in 1.5 ml of pyridine, cooled to ice-bath temperature, and 0.4 ml of acetic anhydride was added. The reaction mixture was kept at room temperature for 7 hours and then for 23 hours in a refrigerator. It was then diluted with 10 ml water and extracted with 30 ml of ethyl acetate. The organic extract was washed with water and brine, dried over sodium sulfate and evaporated. The residue was FLASH chromatographed on a 10×140 mm column with 1:6 and 1:4 ethyl acetate-hexane as the mobile phase to give 126 mg of 1,3-Di-O-acetyl-1,25-Dihydroxy-16-ene-23-yne-26,27-hexafluoro-19-nor-cholecalciferol (2), and 248 mg of 1,3,25-Tri-O-acetyl-1,25-Dihydroxy-16-ene-23-yne-26,27-hexafluoro-19-nor-cholecalciferol (3).

### Example 3

### Synthesis of 1,3-Di-O-acetyl-1,25-dihydroxy-16-ene-23-yne-cholecalciferol (4)

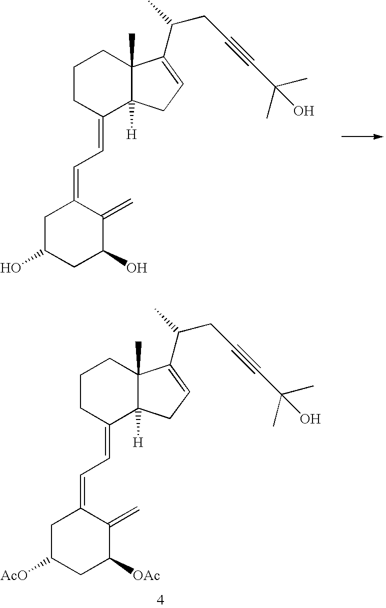

A 10-mL round-bottom flask was charged with 40 mg of 1,25-dihydroxy-16-ene-23-yne-cholecalciferol. This material was dissolved in 1 mL of pyridine. This solution was cooled in an ice bath then 0.3 mL of acetic anhydride was added. The solution was stirred for 30 min, then refrigerated overnight, diluted with water and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-5, 1:6 for fractions 6-13 and 1:4 ethyl acetate-hexane for fractions 14-20 (18 mL fractions). Fractions 14-19 contained the main band with Rf 0.15 (TLC 1:4). Those fractions were pooled and evaporated to a colorless oil, 0.044 g. The material was taken up in methyl formate, filtered and evaporated to give a colorless, sticky foam, 0.0414 g of the title compound (4).

### Example 4

### Synthesis of 1,3-Di-O-acetyl-1,25-dihydroxy-16,23E-diene-cholecalciferol (5)

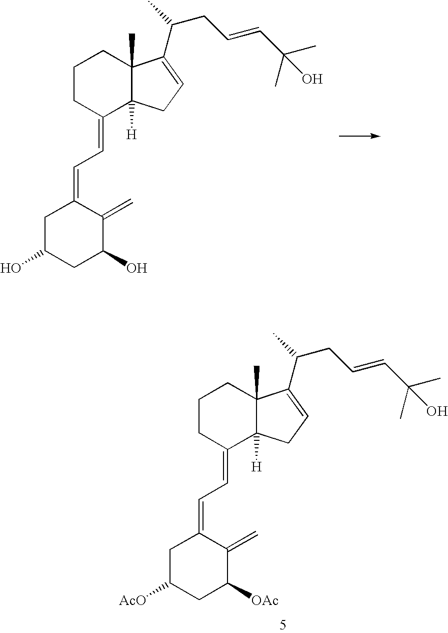

0.0468 g of 1,25-Dihydroxy-16,23E-diene-cholecalciferol was dissolved in 1.5 mL of pyridine. This solution was cooled in an ice bath then refrigerated overnight, diluted with 10 μL of water while still immersed in the ice bath, stirred for 10 min and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-3 (20 mL fractions), 1:6 for fractions 6-8 and 1:4 ethyl acetate-hexane for fractions 9-17 (18 mL each). Fractions 11-14 contained the main band with Rf 0.09 (TLC 1:4). Those fractions were pooled and evaporated to a colorless oil, 0.0153 g. This material was taken up in methyl formate, filtered and evaporated, to give 0.014 g of the title compound (5).

### Example 5

### Synthesis of 1,3-Di-O-acetyl-1,25-dihydroxy-16-ene-cholecalciferol (6)

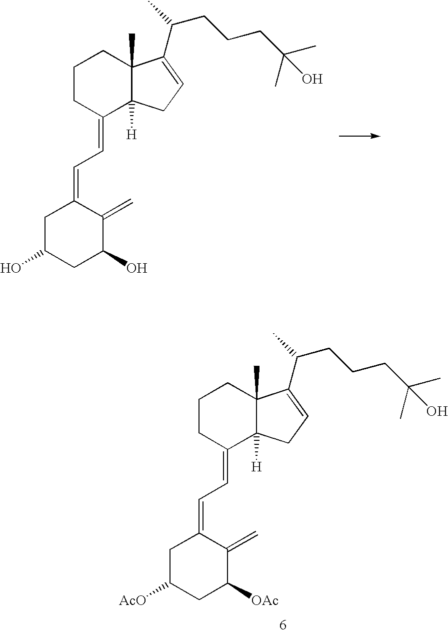

0.0774 g of 1,25-Dihydroxy-16-ene-cholecalciferol was dissolved in 1.5 mL of pyridine. This solution was cooled in an ice bath then 0.3 mL of acetic anhydride was added. The solution was stirred, refrigerated overnight then diluted with 1 mL of water, stirred for 1 h in the ice bath and diluted with 30 mL of ethyl acetate and 15 mL of water. The organic layer was washed with 4×15 mL of water, once with 5 μL of brine then dried (sodium sulfate) and evaporated. The light brown, oily residue was taken up in 1:9 ethyl acetate-hexane then flash chromatographed on a 10×130 mm column using 1:9 ethyl acetate-hexane as mobile phase for fraction 1 (20 mL fractions), 1:6 for fractions 2-7 and 1:4 ethyl acetate-hexane for fractions 8-13. Fractions 9-11 contained the main band with Rf 0.09 (TLC 1:4 ethyl acetate-hexane). Those fractions were pooled and evaporated to a colorless oil, 0.0354 g. This material was taken up in methyl formate, filtered and the solution evaporated, 0.027 g colorless film, the title compound (6).

### Example 6

### Synthesis of 1,3,25-Tri-O-acetyl-1,25-dihydroxy-16-ene-23-yne-26,27-hexafluoro-cholecalciferol (7) and 1,3-Di-O-acetyl-1,25-dihydroxy-16-ene-23-yne-26,27-hexafluoro-cholecalciferol (8)

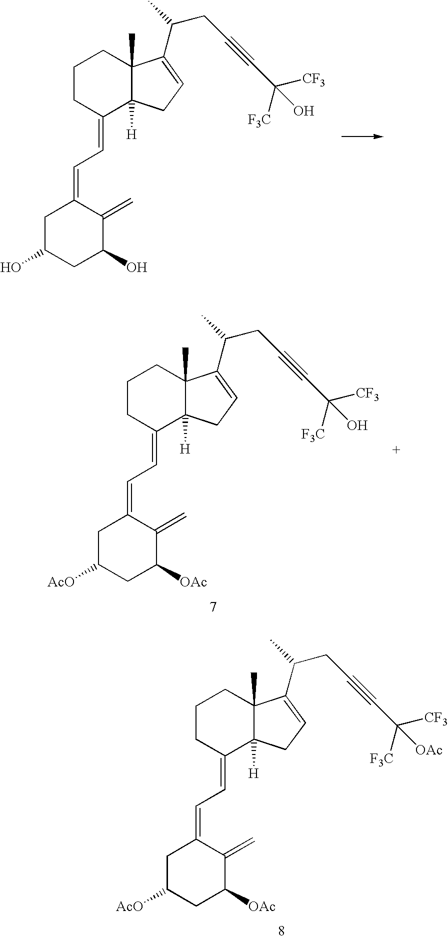

0.0291 g of 1,25-dihydroxy-16-ene-23-yne-26,27-hexafluoro-cholecalciferol was dissolved in 1.5 mL of pyridine. This solution was cooled in an ice bath then 0.25 m, of acetic anhydride was added. The solution was stirred for 20 min and kept in a freezer overnight. The cold solution was diluted with 15 mL of water, stirred for 10 min, and diluted with 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×110 mm column using 1:6 ethyl acetate-hexane as mobile phase. Fractions 2-3 gave 72.3461−72.3285=0.0176 g. Evaporation of fractions 6-7 gave 0.0055 g. The residue of fractions 2-3 was taken up in methyl formate, filtered and evaporated to give 0.0107 g of the title triacetate (7). The residue of fractions 6-7 was taken up in methyl formate, filtered and evaporated to give 0.0049 g of diacetate (8).

### Example 7

### Synthesis of 1,3-Di-O-acetyl-1,25-dihydroxy-16,23E-diene-25R,26-trifluoro-cholecalciferol (9)

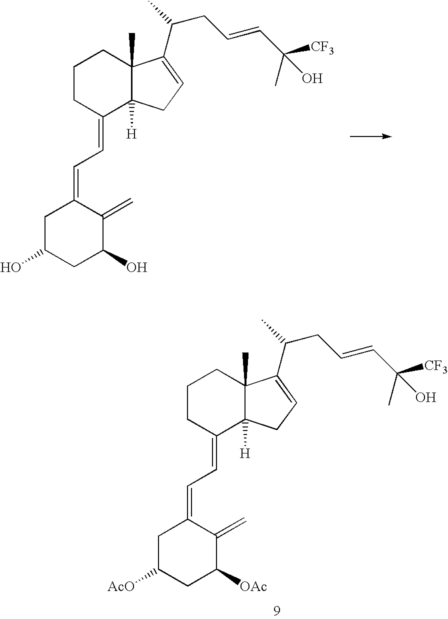

1.5 mL of 1,25-dihydroxy-16,23E-diene-25R,26-trifluoro-cholecalciferol was dissolved in 1.5 mL of pyridine, cooled to ice-bath temperature and 0.4 mL of acetic anhydride was added. The mixture was then refrigerated. After two days the mixture was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×130 mm column using 1:6 ethyl acetate-hexane as mobile phase. Fractions 4-6 (TLC, 1:4) contained the main band (see TLC) These fractions were evaporated and gave 0.0726 g. This residue was taken up in methyl formate, filtered and evaporated, to give 0.0649 g of colorless foam, the title compound (9).

### Example 8

### Synthesis of 1,3-Di-O-acetyl-1,25-dihydroxy-16-ene-19-nor-cholecalciferol (10)

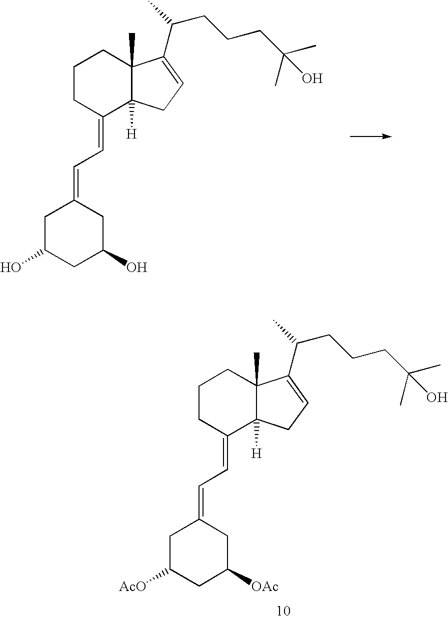

0.0535 g of 1,25-Dihydroxy-16-ene-19-nor-cholecalciferol was dissolved in 1.5 mL of pyridine, cooled to ice-bath temperature and 0.3 μL of acetic anhydride was added and the mixture was refrigerated overnight. The solution was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The nearly colorless, oily residue was taken up in 1:6 ethyl acetate-hexane as mobile phase for fractions 1-6 then 1:4 ethyl acetate-hexane was used. Fractions 9-19 (TLC, 1:4 ethyl acetate-hexane, Rf 0.09, see below) were pooled, evaporated, to give 0.0306 g, which was taken up in methyl formate, filtered, then evaporated. It gave 0.0376 of the title compound (10).

### Example 9

### Synthesis of 1,3-Di-O-Acetyl-1,25-dihydroxy-16-ene-23-yne-19-nor-cholecalciferol (11)

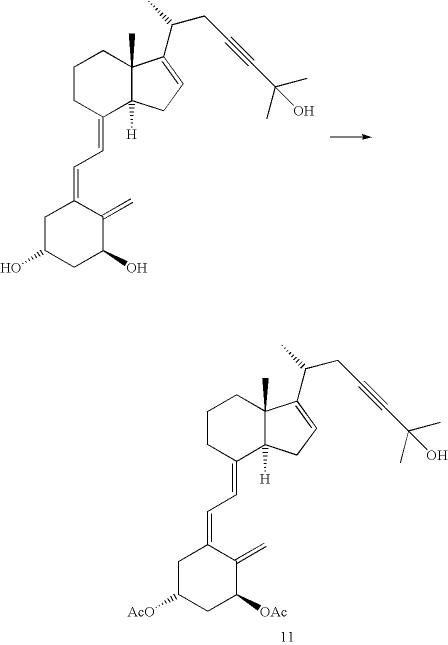

50 nm of 1,25-dihydroxy-16-ene-23-yne-19-nor-cholecalciferol was dissolved in 0.8 mL of pyridine, cooled to ice-bath temperature and 0.2 mL of acetic anhydride was added. The mixture was refrigerated for 3 days then diluted with 1 mL of water, stirred for 10 min in the ice bath and distributed between 5 mL of water and 20 mL of ethyl acetate. The organic layer was washed with 4×5 mL of water, once with 3 mL of brine then dried (sodium sulfate) and evaporated. The nearly colorless, oily residue was taken up in 1:6 ethyl acetate-hexane then flash chromatographed on a 15×120 mm column using 1:6 ethyl acetate-hexane as mobile phase for fractions 1-6, 1:4 for fractions 9-12, 1:3 for fractions 13-15 and 1:2 ethyl acetate-hexane for the remaining fractions. Fractions 11-16 (TLC, 1:4 ethyl acetate-hexane, Rf 0.09, see below) were pooled, evaporated 76.1487−76.1260=0.0227 g, taken up in methyl formate, filtered, then evaporated. It gave 0.0186 g of the title compound (11).

### Example 10

### Synthesis of 1,3-Di-O-acetyl-1,25-dihydroxy-16-ene-23-yne-26,27-bishomo-19-nor-cholecalciferol (12)

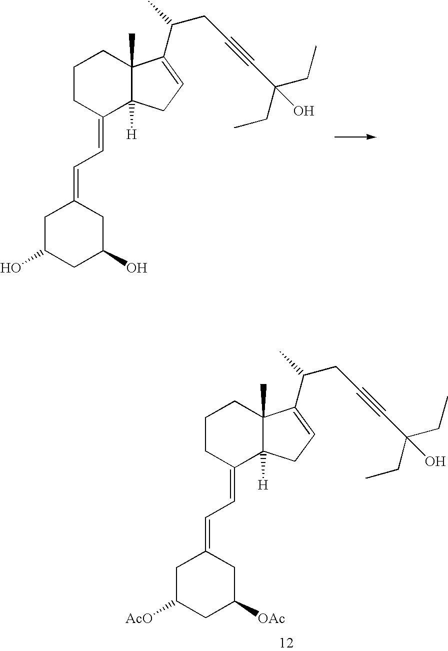

0.0726 g of 1,25-dihydroxy-16-ene-23-yne-26,27-bishomo-19-nor-cholecalciferol was dissolved in 0.8 mL of pyridine, cooled to ice-bath temperature and 0.2 mL of acetic anhydride was added. The solution was stirred in the ice-bath then refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, 33.5512−33.4654=0.0858 g of a tan oily residue that was flash-chromatographed on a 15×120 mm column using 1:6 as mobile phase. Fractions 7-11 (20 mL each) were pooled (TLC 1:4 ethyl acetate-hexane, Rf 0.14) and evaporated, 67.2834−67.2654=0.018 g. This residue was taken up in methyl formate, filtered and evaporated. It gave 0.0211 g of the title compound (12).

### Example 11

### Synthesis of 1,3-Di-O-acetyl-1,25-dihydroxy-20-cyclopropyl-23-yne-19-nor-cholecalciferol (13)

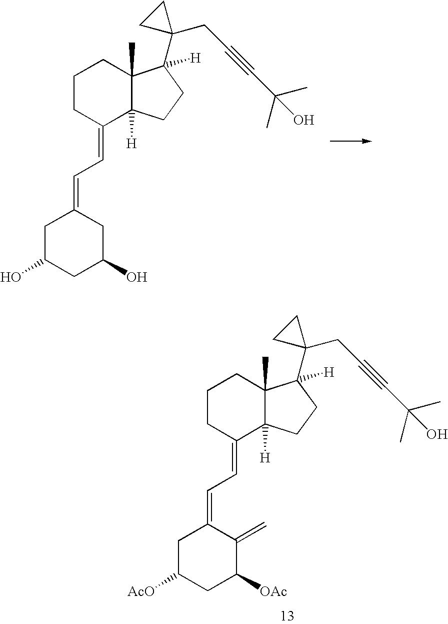

0.282 g of 1,25-Dihydroxy-20-cyclopropyl-23-yne-19-nor-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 mm in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×110 mm column using 1:6 ethyl acetate-hexane as mobile phase for fractions 1-4, 1:4 for fractions 5-12, 1:3 for fractions 13-15 ethyl acetate-hexane for the remaining fractions. Fractions 7-12 (TLC, 1:4 ethyl acetate-hexane, Rf 0.13) were pooled, evaporated, the residue taken up in methyl formate, filtered, then evaporated to give 0.023 g of the title compound (13).

### Example 12

### Synthesis of 1,3,25-Tri-O-acetyl-1,25-dihydro-20-cyclopropyl-23-yne-26,27-hexafluoro-19-nor-cholecalciferol (14) and 1,3-Di-O-acetyl-1,25-dihydroxy-20-cyclopropyl-23-yne-26,27-hexafluoro-19-nor-cholecalciferol (15)

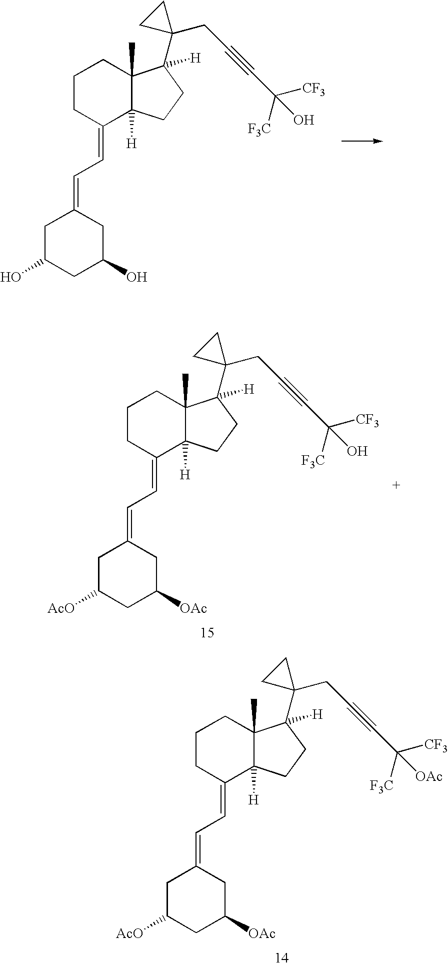

0.1503 g of 1,25-dihydroxy-20-cyclopropyl-23-yne-26,27-hexafluoro-19-nor-cholecalciferol was dissolved in 0.8 mL of pyridine, cooled to ice-bath temperature and 0.2 mL of acetic anhydride was added. The mixture was refrigerated overnight then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×150 mm column using 1:6 ethyl acetate-hexane as mobile phase for fractions 1-5, 1:4 for the remaining fractions. Fractions 3-4 and 6-7 were pooled, evaporated, then taken up in methyl formate, filtered, and evaporated to give 0.0476 g of the title triacetate (14) and 0.04670 g of the title diacetate (15).

### Example 13

### Synthesis of 1,3-Di-O-acetyl-1,25-dihydroxy-20-cyclopropyl-23-yne-cholecalciferol (16)

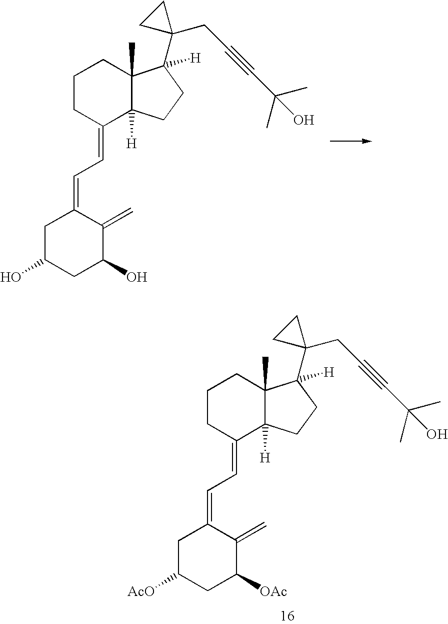

0.0369 g of 1,25-dihydroxy-20-cyclopropyl-23-yne-cholecalciferol was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash-chromatographed on a 13×110 mm column using 1:6 ethyl acetate-hexane as mobile phase for fractions 1-7, 1:4 ethyl acetate-hexane for the remaining fractions. Fractions 9-11 (TLC, 1:4 ethyl acetate-hexane) were pooled, evaporated, taken up in methyl formate, filtered, then evaporated, to give 0.0099 g of the title compound (16).

### Example 14

### Synthesis of 1,3-Di-O-acetyl-1,25-dihydroxy-20-cyclopropyl-23E-ene-26,27-hexafluoro-19-nor-cholecalciferol (17)

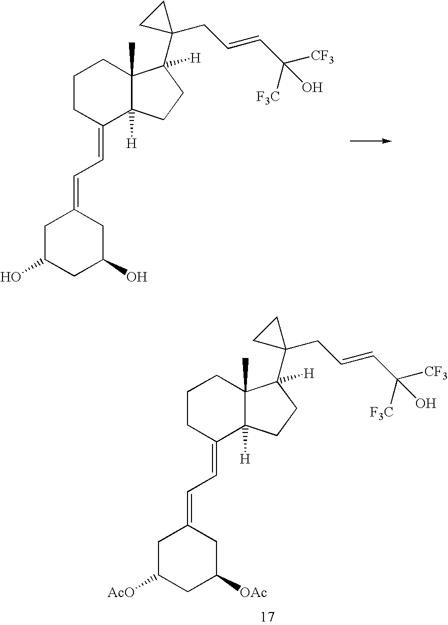

0.0328 g of 1,25-dihydroxy-20-cyclopropyl-23E-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 5 mL of water and 20 mL of ethyl acetate. (Extraction of the aqueous layer gave no phosphomolybdic acid-detectable material). The organic layer was washed with 3×5 mL of water, once with 5 mL of saturated sodium hydrogen carbonate, once with 3 mL of brine then dried (sodium sulfate) and evaporated, the residue shows Rf 0.25 as the only spot. The oily residue was taken up in 1:6 ethyl acetate-hexane then flash-chromato-graphed on a 13.5×110 mm column using 1:6 ethyl acetate-hexane as mobile phase for fractions 1-10. Fractions 4-9 were pooled and evaporated, the residue taken up in methyl formate, filtered, then evaporated to give 0.0316 g of the title compound (1.7).

### Example 15

### Synthesis of 1,3-Di-O-acetyl-1,25-dihydroxy-20-cyclopropyl-23Z-ene-26,27-hexafluoro-[9-nor-cholecalciferol (18)

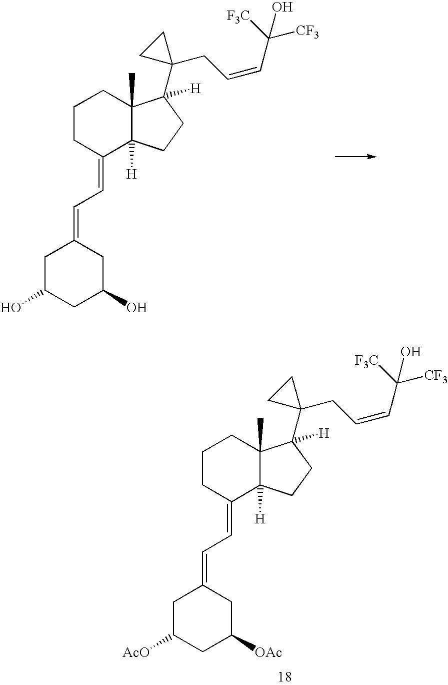

0.0429 g of 1,25-dihydroxy-20-cyclopropyl-23Z-ene-26,27-hexafluoro-19-nor-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 7 mL of water and 25 mL of ethyl acetate. The organic layer was washed with 3×5 mL of water, once with 5 mL of saturated sodium hydrogen carbonate, once with 3 mL of brine then dried (sodium sulfate, TLC (1:4 ethyl acetate-hexane shows mostly one spot) and evaporated, flash-chromatographed on a 15×120 mm column using 1:6 as mobile phase. Fractions 3-6 (20 mL each) were pooled and evaporated. The residue was taken up in methyl formate, filtered and evaporated, to give 0.0411 g of the title compound (18).

### Example 16

### Synthesis of 1,3-Di-O-acetyl-1,25-dihydroxy-20-cyclopropyl-cholecalciferol (19)

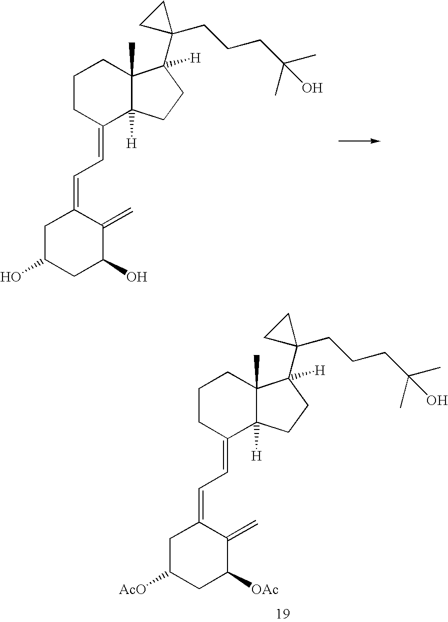

0.0797 g of 1,25-dihydroxy-20-cyclopropyl-cholecalciferol was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, to give 0.1061 g of a tan oily residue that was flash-chromatographed on a 15×120 mm column using 1:6 as mobile phase. Fractions 9-16 (20 mL each) were pooled (TLC 1:4 ethyl acetate-hexane, Rf 0.13) and evaporated. This residue was taken up in methyl formate, filtered and evaporated to give 0.0581 g of the title compound (19).

### Example 17

### Synthesis of 1,3-Di-O-acetyl-1α,25-dihydroxy-16-ene-20-cyclopropyl-19-nor-cholecalciferol (20)

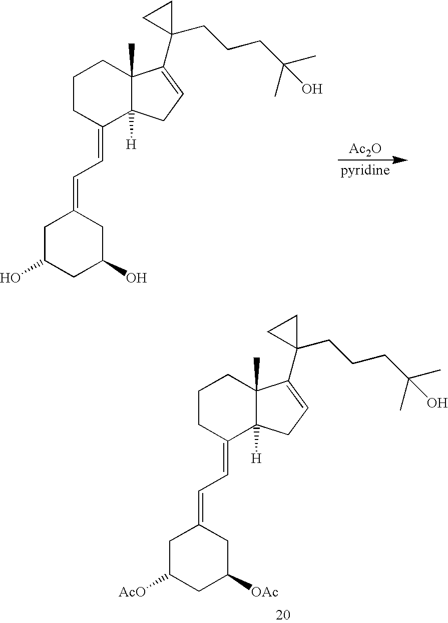

To the solution of 1α,25-Dihydroxy-16-ene-20-cyclopropyl-19-nor-cholecalciferol (94 mg, 0.23 mmol) in pyridine (3 mL) at 0° C., acetic anhydride (0.5 mL, 5.3 mmol) was added. The mixture was stirred for 1 h, refrigerated for 15 h. and then was stirred for additional 8 h. Water (10 mL) was added and after stirring for 15 min. the reaction mixture was extracted with AcOEt:Hexane 1:1 (25 mL), washed with water (4×25 mL) and brine (20 mL), dried over Na₂SO₄. The residue (120 mg) after evaporation of the solvent was purified by FC (15 g, 30% AcOEt in hexane) to give the titled compound (20) (91 mg, 0.18 mmol, 80%).

[α]³⁰_{D}=+14.4 c 0.34, EtOH

UV λmax (EtOH): 242 nm (E 34349) 250 nm (c 40458), 260 nm (ε27545);

¹H NMR (CDCl₃): 6.25 (1H, d, J=11.1 Hz), 5.83 (1H, d, J=11.3 Hz), 5.35 (1H, m), 5.09 (2H, m), 2.82-1.98 (7H, m), 2.03 (3H, s), 1.98 (3H, s), 2.00-1.12 (15H, m), 1.18 (6H, s), 0.77 (3H, s), 0.80-0.36 (4H, m);

¹³C NMR (CDCl₃): 170.73(0), 170.65(0), 157.27(0), 142.55(0), 130.01(0), 125.06(1), 123.84(1), 115.71(1), 71.32(0), 70.24(1), 69.99(1), 59.68(1), 50.40(0), 44.08(2), 41.40(2), 38.37(2), 35.96(2), 35.80(2), 32.93(2), 29.48(3), 29.31(2), 28.71(2), 23.71(2), 22.50(2), 21.56(3), 21.51(0), 21.44(3), 18.01(3), 12.93(2), 10.53(2);

| | | | |
|---|---|---|---|
| MS HRES | Calculated for C₃₁H₄₆O₅ | M + Na | 521.3237 |
| | Observed | M + Na | 521.3233 |

### Example 18

### Synthesis of 1,3-Di-acetyl-1α,25-hydroxy-16-ene-20-cyclopropyl-cholecalciferol (21)

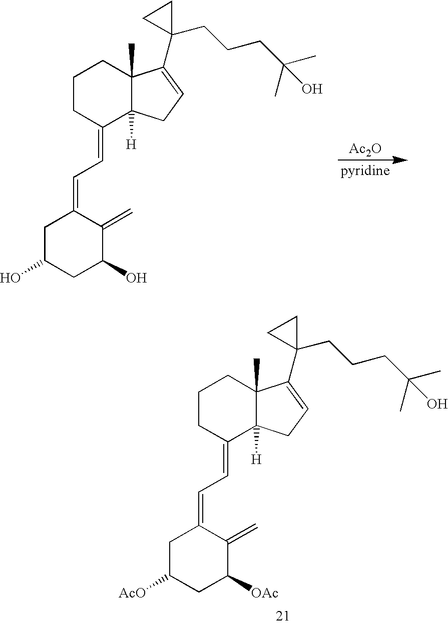

To the solution of 1α,25-Dihydroxy-16-ene-20-cyclopropyl-cholecalciferol (100 mg, 0.23 mmol) in pyridine (3 mL) at 0° C., acetic anhydride (0.5 mL, 5.3 mmol) was added. The mixture was stirred for 2 h and then refrigerated for additional 15 h. Water (10 mL) was added and after stirring, for 15 min. the reaction mixture was extracted with AcOEt:Hexane 1:1 (25 mL), washed with water (4×25 mL), brine (20 mL) and dried over Na₂SO₄. The residue (150 mg) after evaporation of the solvent was purified by FC (15 g, 30% AcOEt in hexane) to give the titled compound (21) (92 mg, 0.118 mmol, 78%)

[α]_{D}=−14.9 c 0.37, EtOH

UV λmax (EtOH): 208 nm (ε15949), 265 nm (ε15745);

¹H NMR (CDCl₃): 6.34 (1H, d, J=11.3 Hz), 5.99 (1H, d, J=11.3 Hz), 5.47 (11H, m), 5.33 (1H, m), 5.31 (1H, s), 5.18 (1H, m), 5.04 (11H, s), 2.78 (11H, m), 2.64 (1H, m), 2.40-1.10 (18H, m), 2.05 (3H, s), 2.01 (3H, s), 1.18 (6H, s), 0.76 (3H, s), 0.66-0.24 (4H, m);

¹³C NMR (CDCl₃): 170.76(0), 170.22(0), 157.18(0), 143.02(0), 142.40(0), 131.94(0), 125.31(1), 125.10(1), 117.40(1), 115.22(2), 72.97(1), 71.32(0), 69.65(1), 59.71(1), 50.57(0), 44.07(2), 41.73(2), 38.36(2), 37.10(2), 35.80(2), 29.45(3), 29.35(2), 29.25(3), 28.92(2), 23.80(2), 22.48(2), 21.55(3), 21.50(3), 21.35(0), 17.90(3), 12.92(2), 10.54(2);

| | | | |
|---|---|---|---|
| MS HRES | Calculated for C₃₂H₄₆O₅ | M + Na | 533.3237 |
| | Observed | M + Na | 533.3236 |

### Example 19

### Synthesis of 1,3-Di-O-acetyl-1,25-dihydroxy-23-yne-cholecalciferol (22)

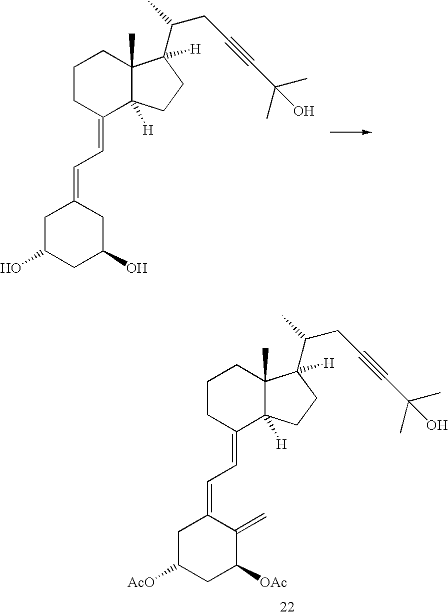

0.2007 g of (0.486 mmol) was dissolved in 2 mL of pyridine. This solution was cooled in an ice bath and 0.6 mL of acetic anhydride was added. The solution was kept in an ice bath for 45 h then diluted with 10 mL of water, stirred for 10 min and equilibrated with 10 mL of water and 40 mL of ethyl acetate. The organic layer was washed with 4×20 mL of water, 10 mL of brine, dried (sodium sulfate) and evaporated. The brown, oily residue was flash chromatographed using 1:19, 1:9, and 1:4 ethyl acetate-hexane as stepwise gradient. The main band with Rf 0.16 (TLC 1:4 acetate-hexane) was evaporated to give 1,3-di-O-acetyl-1,25-dihydroxy-23-yne-cholecalciferol (22) a colorless foam, 0.0939 g.

### Example 20

### Synthesis of (3aR,4S,7aR)-7a-Methyl-1-[1-(4-hydroxy-4-methyl-pent-2-ynyl)-cyclopropyl]-3a,4,5,6,7,7a-hexahydro-3H-inden-4-ol

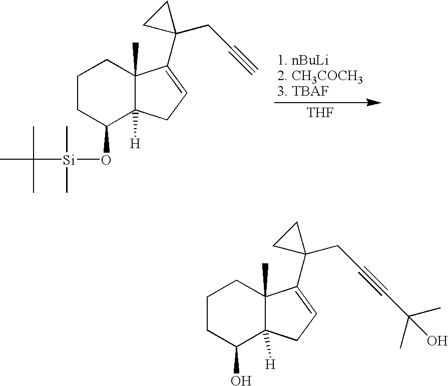

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.0 g, 2.90 mmol) in tetrahydrofurane (15 mL) at −78° C. was added n-BuLi (2.72 mL, 4.35 mmol, 1.6M in hexane). After stirring at −78° C. for 1 h., acetone (2.5 mL, 34.6 mmol) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-2-methyl-pent-3-yn-2-ol (1.05 g, 2.61 mmol) which was treated with tetrabutylammonium fluoride (6 mL, 6 mmol, 11.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄. The residue after evaporation of the solvent (1.1 g) was purified by FC (50 g, 20% AcOEt in hexane) to give the titled compound (0.75 g, 2.59 mmol, 90%). [α]³⁰_{D}=+2.7 c 0.75, CHCl₃. ¹H NMR (CDCl₃): 5.50 (1H, m), 4.18 (1H, m), 2.40 (2H, s), 2.35-1.16 (1H, m), 1.48 (6H, s), 1.20 (3H, s), 0.76-0.50 (4H, m); ¹³C NMR (CDCl₃): 156.39, 125.26, 86.39, 80.19, 69.21, 65.16, 55.14, 46.94, 35.79, 33.60, 31.67, 29.91, 27.22, 19.32, 19.19, 17.73, 10.94, 10.37; MS HREI Calculated for C₂₂H₂₈O₂ M+288.2089 Observed M+288.2091.

### Example 21

### Synthesis of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol

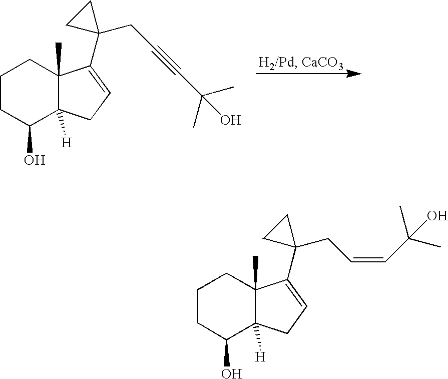

The mixture of (3aR,4S,7aR)-7a-Methyl-1-[1-(−4-hydroxy-4-methyl-pent-2-ynyl)-cyclopropyl]-3a,4,5,6,7,7a-hexahydro-3H-inden-4-ol (0.72 g, 2.50 mmol), ethyl acetate (10 mL), hexane (24 mL), absolute ethanol (0.9 mL), quinoline (47 uL) and Lindlar catalyst (156 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (0.79 g) was purified by FC (45 g, 20% AcOEt in hexane) to give the titled compound (640 mg, 2.2 mmol, 88%).

### Example 22

### Synthesis of (3aR,4S,7aR)-7a-Methyl-1-[1-(4-hydroxy-4-methyl-pentyl)-cyclopropyl]-3a,4,5,6,7,7a-hexahydro-3H-inden-4-ol

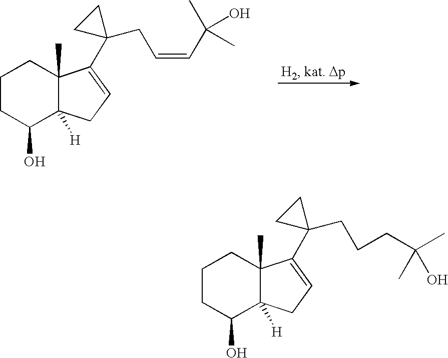

The mixture of (3aR,4S,7aR)-7a-Methyl-1-[1-(4-hydroxy-4-methyl-pent-2Z-enyl)-cyclopropyl]-3a,4,5,6,7,7a-hexahydro-3H-inden-4-ol (100 mg, 0.34 mmol), 1,4-bis(diphenyl-phosphino)butane 1,5 cyclooctadiene rhodium tetrafluoroborate (25 mg, 0.034 mmol), dichloromethane (5 mL) and one drop of mercury was hydrogenated using Paar apparatus at room temperature and 50 p.s.i. pressure for 3 h. The reaction mixture was filtered through Celite pad, which was then washed with ethyl acetate. The combine filtrates and washes were evaporated to dryness (110 mg) and purified by FC (10 g, 20% AcOEt in hexane) to give the titled compound (75 mg, 0.26 mmol, 75%). [α]³⁰_{D}=−8.5 c 0.65, CHCl₃. ¹H NMR (CDCl₃): 5.37 (1H, m), 4.14 (1H, m), 2.37-1.16 (17H, m), 1.19 (6H, s), 1.18 (3H, s), 0.66-0.24 (4H, m); MS HREI Calculated for C₁₉H₃₂O₂ M+H 292.2402. Observed M+H 292.2404.

### Example 23

### Synthesis of (3aR,7aR)-7a-Methyl-1-[1-(4-methyl-4-trimethylsilanyloxy-pentyl)-cyclopropyl]-3a,4,5,6,7,7a-hexahydro-3H-inden-4-one

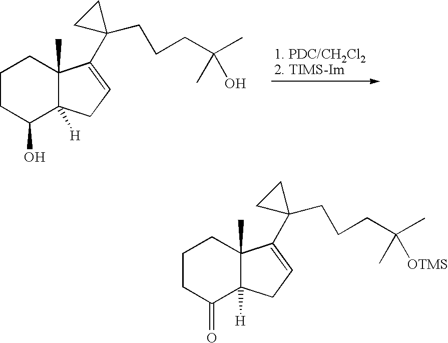

To a stirred suspension of (3aR,4S,7aR)-7a-Methyl-1-[1-(4-hydroxy-4-methyl-pentenyl)-cyclopropyl]-3a,4,5,6,7,7a-hexahydro-3H-inden-4-ol (440 mg, 1.50 mmol) and Celite (2.0 g) in dichloromethane (10 mL) at room temperature wad added pyridinium dichromate (1.13 g, 3.0 mmol). The resulting mixture was stirred for 5 h filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pentenyl)-cyclopropyl]-3a,4,5,6,7,7a-hexahydro-3H-inden-4-one (426 mg, 1.47 mmol, 98%). To a stirred solution of (3aR,7aR)-7a-Methyl-1-[1-(4-hydroxy-4-methyl-pentenyl)-cyclopropyl]-3a,4,5,6,7,7a-hexahydro-3H-inden-4-one (424 mg, 1.47 mmol) in dichloromethane (10 mL) at room temperature was added trimethylsilyl-imidazole (0.44 mL, 3.0 mmol). The resulting mixture was stirred for 1.0 h filtered through silica gel (10 g) and the silica gel pad was washed with 10% AcOEt in hexane. Combined filtered and washes were evaporated to give the titled compound (460 mg, 1.27 mmol, 86%). [α]²⁹_{D}=−9.9 c 0.55, CHCl₃, ¹H NMR (CDCl₃): 5.33 (1H, dd, J=3.2, 1.5 Hz), 2.81 (1H, dd, J=10.7, 6.2 Hz), 2.44 (11, ddd, J=15.6, 10.7, 1.5 Hz), 2.30-1.15(131H, m) overlapping 2.03 (ddd, T=15.8, 6.4, 3.2 Hz), 1.18(6H, s), 0.92(3H, s), 0.66-0.28 (4H, m), 0.08 (9H, s); ¹³C NMR (CDCl₃): 211.08 (0), 155.32(0), 124.77(1), 73.98(0), 64.32(1), 53.91(0), 44.70(2), 40.45(2), 38.12(2), 34.70(2), 29.86(3), 29.80(3), 26.80(2), 24.07(2), 22.28(2), 21.24(0), 18.35(3), 12.60(2), 10.64(2), 2.63 (3); MS HRES Calculated for C₂₂H₃₈O₂Si M+362.2641. Observed M+362.2648.

### Example 24

### Synthesis of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one

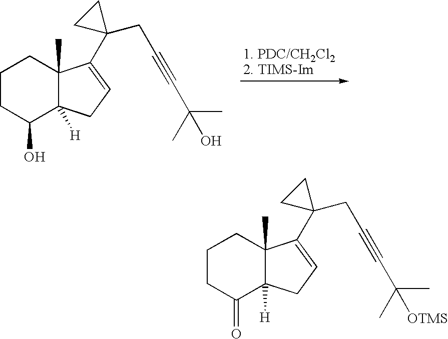

To a stirred suspension of (3aR,4S,7aR)-7a-Methyl-1-[1-(4-hydroxy-4-methyl-pent-2-ynyll)-cyclopropyl]-3a,4,5,6,7,7a-hexahydro-3H-inden-4-ol (381 mg, 1.32 mmol) and Celite (2.0 g) in dichloromethane (10 mL) at room temperature wad added pyridinium dichromate (1.0 g, 2.65 mmol). The resulting mixture was stirred for 1.5 b filtered through silica gel (10 g), and then silica gel pad was washed with 20% AcOEt in hexane. The combined filtrate and washes were evaporated, to give a crude (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol, 95%). To a stirred solution of (3aR,7aR)-7a-Methyl-1-[1-(4-hydroxy-4-methyl-pent-2-ynyll)-cyclopropyl]-3a,4,5,6,7,7a-hexahydro-3H-inden-4-one (360 mg, 1.26 mmol) in dichloromethane (10 mL) at room temperature was added trimethylsilyl-imidazole (0.25 mL, 1.7 mmol). The resulting mixture was stirred for 0.5 h filtered through silica gel (10 g) and the silica gel pad was washed with 5% AcOEt in hexane. Combined filtered and washes were evaporated to give the titled compound (382 mg, 1.07 mmol, 81%).

### Example 25

### Synthesis of 1a,25-Dihydroxy-16-ene-20-cyclopropyl-23,24-yne-cholecalciferol (23)

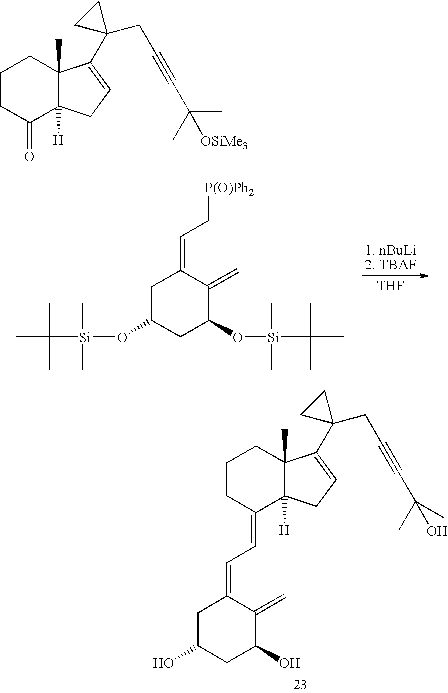

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (513 mg, 0.88 mmol) in tetrahydrofurane (6 mL) at −78° C. was added n-BuLi (0.55 mL, 0.88 mmol). The resulting mixture was stirred for 15 min and solution of (3aR,7aR)-7a-Methyl-1-[1-(4-methyl-4-trimethylsilanyloxy-pent-2-ynyll)-cyclopropyl]-3a,4,5,6,7,7a-hexahydro-3H-inden-4-one (179 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (716 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-23,24-yne-cholecalciferol (324 mg, 045 mmol). To the 1α,3β-Di(tert-Butyl-dimethyl-silanyloxy)-25-trimethylsilanyloxy-16-ene-20-cyclopropyl-23,24-yne-cholecalciferol (322 mg, 0.45 mmol) tetrabutylammonium fluoride (4 mL, 4 mmol, 1M solution in THF) was added, at room temperature. The mixture was stirred for 18 k diluted with AcOEt (25 mL) and washed with water (5×20 mL), brine (20 mL) and dried over Na₂SO₄. The residue (280 mg) after evaporation of the solvent was purified by FC (10 g, 50% AcOEt in hexane and AcOEt) to give the titled compound (23) (172 mg, 0.41 mmol, 82%). [α]³¹_{D}=+32.4 c 0.50, MeOH. UV λmax (EtOH): 261 nm (ε11930); ¹H NMR (CDCl₃): 6.36(1H, d, J=11.3 Hz), 6.09 (1H, d, J=11.3 Hz), 5.45 (1H, m), 5.33 (1H, m), 5.01 (1H, s), 4.45 (1H, m), 4.22 (1H, m), 2.80 (1H, m), 2.60 (1H, m), 2.50-1.10 (16H, m), 1.45 (6H, s), 0.81 (3H, s), 0.72-0.50 (4H, m); MS HRES Calculated for C₂₈H₃₈O₃ M+422.2821. Observed M+ 422.2854.

### Example 26

### Synthesis of 1α,25-Dihydroxy-16-ene-20-cyclopropyl-23,24-yne-19-nor-cholecalciferol (24)

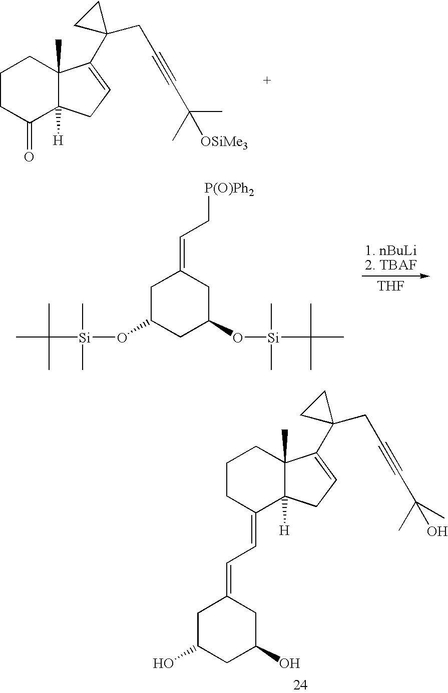

To a stirred solution of a (1R,3R)-1,3-bis-((tert-butyldimethyl)silanyloxy)-5-[2-(diphenylphosphinoyl)ethylidene]-cyclohexane (674 mg, 1.18 mmol) in tetrahydrofurane (8 mL) at −78° C. was added n-BuLi (0.74 mL, 1.18 mmol). The resulting mixture was stirred for 15 min and solution of (3aR,7aR)-7a-Methyl-1-[1-(4-methyl-4-trimethylsilanyloxy-pent-2-ynyl)-cyclopropyl]-3a,4,5,6,7,7a-hexahydro-3H-inden-4-one (235 mg, 0.66 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (15 g, 5% AcOEt in hexane) to give 1α,3β-Di(tert-Butyl-dimethyl-silanyloxy)-25-trimethylsilanyloxy-6-ene-20-cyclopropyl-23,24-yne-19-nor-cholecalciferol (330 mg, 0.46 mmol). To the 1α,3β-Di(tert-Butyl-dimethyl-silanyloxy)-25-trimethylsilanyloxy-16-ene-20-cyclopropyl-23,24-yne-19-nor-cholecalciferol (328 mg, 0.46 mmol) tetrabutylammonium fluoride (5 mL, 5 mmol, 1M solution in THF) was added, at room temperature. The mixture was stirred for 62 h. diluted with AcOEt (25 mL) and washed with water (50 mL), brine (20 mL) and dried over Na₂SO₄. The residue (410 mg) after evaporation of the solvent was purified by FC (10 g, 50% AcOEt in hexane and AcOEt) to give the titled compound (24) (183 mg, 0.45 mmol, 68%). [α]²⁹_{D}=+72.1 c 0.58, MeOH. UV λmax (EtOH): 242 nm (ε29286), 251 nm (ε34518), 260 nm (ε2387 [symbol] ); ¹H NMR (CDCl₃): 6.30 (1H, d, J=11.3 Hz), 5.94 (1H, d, J=11.3 Hz), 5.48 (1H, m), 4.14 (1H, m), 4.07 (1H, m), 2.78 (2H, m), 2.52-1.10 (18H, m), 1.49 (66H, s), 0.81 (3H, s), 0.72-0.50 (4H, m); MS HRES Calculated for C₂₇H₃₈O₃ M+ 410.2821. Observed M+ 410.2823.

### Example 27

### Synthesis of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol

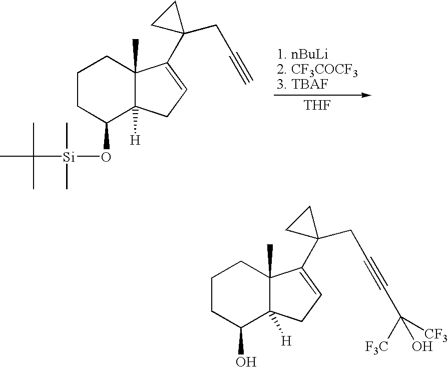

To a stirred solution of (3aR,4S,7aR)-1-{1-[4-(tert-Butyl-dimethyl-silanyloxy)-7a-methyl-3a,4,5,6,7,7a-hexahydro-3H-inden-1-yl])-cyclopropyl}-ethynyl (1.95 g, 5.66 mmol) in tetrahydrofurane (35 mL) at −78° C. was added n-BuLi (4.3 mL, 6.88 mmol, 1.6M in hexane). After stirring at −78° C. for 1 h., hexafluoroacetone (six drops from the cooling finger) was added and the stirring was continued for 1 h. NH₄Cl_{aq} was added (10 mL) and the mixture was allowed to warm to room temperature. The reaction mixture was diluted with brine (100 mL) and extracted with hexane (2×125 mL). The combined extracts were dried over Na₂SO₄. The residue after evaporation of the solvent (8.2 g) was purified by FC (150 g, 10% AcOEt in hexane) to give (3aR,4S,7aR)-5-{1-[4-(tert-Butyl-dimethyl-silanyloxy)-7a-methyl-3a,4,5,6,7,7a-hexahydro-3H-inden-1-yl]-cyclopropyl}-1,1,1-trifluoro-2-trifluoromethyl-pent-3-yn-2-ol (2.73 g, 5.35 mmol) which was treated with tetrabutylammonium fluoride (20 mL, 20 mmol, 1.0M in THF) and stirred at 65-75° C. for 30 h. The mixture was diluted with AcOEt (1150 mL) and washed with water (5×150 mL), brine (150 mL). The combined aqueous washes were extracted with AcOEt (150 mL) and the combined organic extracts were dried over Na₂SO₄. The residue after evaporation of the solvent (3.2 g) was purified by FC (150 g, 20% AcOEt in hexane) to give the titled compound (2.05 g, 5.17 mmol, 97%). [α]²⁸_{D}=+6.0 c 0.47, CHCl₃. ¹H NMR (CDCl₃): 5.50 (1H, br. s), 4.16 (1H, br. s), 3.91 (1H, s), 2.48 (1H, part A of the AB quartet, J=17.5 Hz), 2.43 (11H, part B of the AB quartet, J=17.5 Hz), 2.27 (1H, m), 2.00-1.40 (9H, m), 1.18 (3H, s), 0.8-0.5 (4H, m); ¹³C NMR (CDCl₃): 155.26(0), 126.68(1), 121.32(0, q, J=284 Hz), 90.24 (0), 71.44(0, sep. J=34 Hz), 70.54 (0), 69.57(1), 55.17(1), 47.17(0), 36.05(2), 33.63(2), 30.10(2), 27.94(2), 19.50(3), 19.27(0), 17.90(2), 11.56(2), 11.21(2); MS REI Calculated for C₁₉H₂₂O₂F₆ M+ 396.1524. Observed M+ 396.1513.

### Example 28

### Synthesis of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one

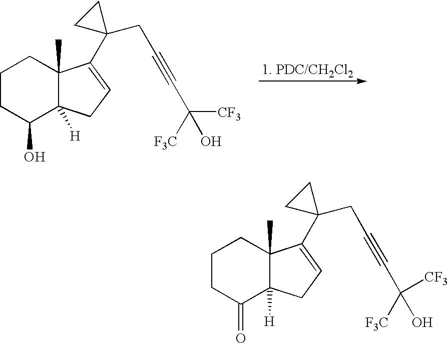

To a stirred suspension of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (504 mg, 1.27 mmol) and Celite (1.5 g) in dichloromethane (12 mL) at room temperature wad added pyridinium dichromate (0.98 g, 2.6 mmol). The resulting mixture was stirred for 2.5 h filtered through silica gel (5 g), and then silica gel pad was washed with 20% AcOEt in hexane. The combined filtrate and washes were evaporated, to give a titled compound (424 mg, 1.08 mmol, 85%). [α]²⁸_{D}=+3.1 c 0.55, CHCl₃. ¹H NMR (CDCl₃): 5.46 (1H, br. s), 3.537 (11H, s), 2.81 (1H, dd, J=10.7, 6.5 Hz), 2.49-1.76 (10H, m), 0.90 (3H, s), 0.77-0.53 (4H, m); MS HREI Calculated for C₁₉H₂₀O₂F₆ M+H 395.1440. Observed M+H 395.1443.

### Example 29

### Synthesis of 14215-Dihydroxy-16-ene-20-cyclopropyl-23,41-yne-26,27-hexafluoro-19-nor-cholecalciferol (25)

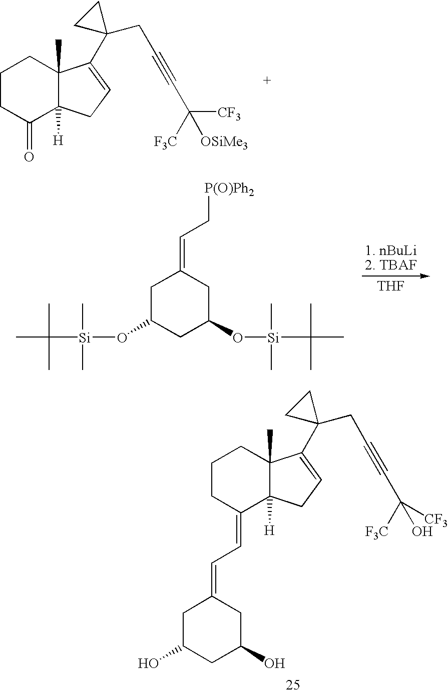

To a stirred solution of a (1R,3R)-1,3-bis-((tert-butyldimethyl)silanyloxy)-5-[2-(diphenylphosphinoyl)ethylidene]-cyclohexane (900 mg, 1.58 mmol) in tetrahydrofurane (8 mL) at −78° C. was added n-BuLi (1.0 mL, 1.6 mmol). The resulting mixture was stirred for 15 min. and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (200 mg, 0.51 mmol, in tetrahydrofurane (3 mL) was added dropwise. The reaction mixture was stirred at −72° C. for 3.5 h diluted with hexane (25 mL) washed brine (30 mL) and dried over Na₂SO₄. The residue (850 mg) after evaporation of the solvent was purified by FC (20 g, 10% AcOEt in hexane) to give 1α,3β-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol, 86%). To the 1α,3β-Di(tert-Butyl-dimethyl-silanyloxy)-25-hydroxy-16-ene-20-cyclopropyl-23,24-yne-26,27-hexafluoro-19-nor-cholecalciferol (327 mg, 0.44 mmol). Tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (250 mg) after evaporation of the solvent was purified by FC (10 g, 50% AcOEt in hexane and AcOEt) to give the titled compound (25) (183 mg, 0.45 mmol, 68%). [α]³⁰_{D}=+73.3 c 0.51, EtOH. UV λmax (EtOH): 243 nm (ε29384). 251 nm (ε34973), 260 nm (ε23924); ¹H NMR (CDCl₃): 6.29 (1H, d, J=11.1 Hz), 5.93 (1H, d, J=11.1 Hz), 5.50 (1H, m), 4.12 (1H, m), 4.05 (1H, m), 2.76 (2H, m), 2.55-1.52 (18H, m), 0.80 (3H, s), 0.80-0.49 (4H, m); ¹³C NMR (CDCl₃): 155.24(0), 141.78(0), 131.28(0), 126.23(1), 123.65(1), 121.09(0, q, J=285 Hz), 115.67(1), 89.63(0), 70.42(0), 67.48(1), 67.29(1), 59.19(1), 49.87(0), 44.49(2), 41.98(2), 37.14(2), 35.76(2), 29.22(2), 28.47(2), 27.57(2), 23.46(2), 19.32(0), 17.97(3), 11.89(2), 10.18(2); MS HRES Calculated for C₂₇H₃₂O₃F₆ M+H 519.2329. Observed M+H 519.2325.

### Example 30

### Synthesis of 1,25-Dihydroxy-16-ene-20-cyclopropyl-23,24-yne-26,27 hexafluoro-cholecalciferol (26)

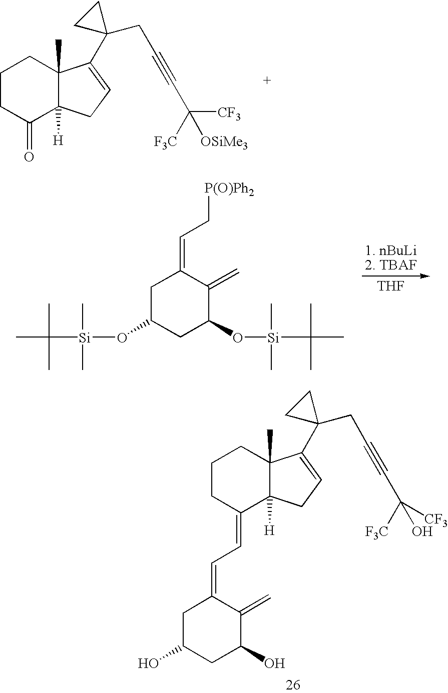

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (921 mg, 1.58 mmol) in tetrahydrofurane (8 mL) at −78° C. was added n-BuLi (1.0 mL, 1.6 mmol). The resulting mixture was stirred for 15 min and solution of (3aR,7aR)-7a-Methyl-1-[1-(5,5,5-trifluoro-4-trifluoromethyl-4-hydroxy-pen-2-ynyl)-cyclopropyl]-3a,4,5,6,7,7a-hexahydro-3H-inden-4-one (197 mg, 0.50 mmol, in tetrahydrofurane (2 mL) was added dropwise. The reaction mixture was stirred at −72° C. for 3.5 h diluted with hexane (25 mL) washed brine (30 mL) and dried over Na₂SO₄. The residue (876 mg) after evaporation of the solvent was purified by FC (20 g, 105% AcOEt in hexane) to give 1α,3β-Di(tert-Butyl-dimethyl-silanyloxy)-25-hydroxy-16-ene-20-cyclopropyl-23,24-yne-26,27-hexafluoro-cholecalciferol (356 mg, 0.47 mmol). To the 1α,3β-Di(tert-Butyl-dimethyl-silanyloxy)-25-hydroxy-16-ene-20-cyclopropyl-23,24-yne-26,27-hexafluoro-cholecalciferol (356 mg, 0.47 mmol) tetrabutylammonium fluoride (5 mL, 5 mmol, 1M solution in THF) was added, at room temperature. The mixture was stirred for 15 h. diluted with AcOEt (25 mL) and washed with water (5×20 nit), brine (20 mL) and dried over Na₂SO₄. The residue (270 mg) after evaporation of the solvent was purified by FC (20 g, 50% AcOEt in hexane and AcOEt) to give the titled compound (26) (216 mg, 0.41 mmol, 87%). [α]³⁰_{D}=+40.0 c 0.53, EtOH. UV λmax (EtOH): 262 nm (ε12919); ¹H NMR (CDCl₃): 6.38 (1H, d, J=11.5 Hz), 6.10 (1H, d, J=11.1 Hz), 5.49 (1H, m), 5.35 (1H, s), 5.02 (1H, s), 4.45 (1H, m), 4.25 (1H, m), 3.57 (1H, s), 2.83-1.45 (18H, m), 0.82 (3H, s), 0.80-0.51 (4H, m); MS HRES Calculated for C₂₈H₃₂O₃F₆ M+H 531.2329. Observed M+H 531.2337.

### Example 31

### Synthesis of (3aR,4S,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-ol

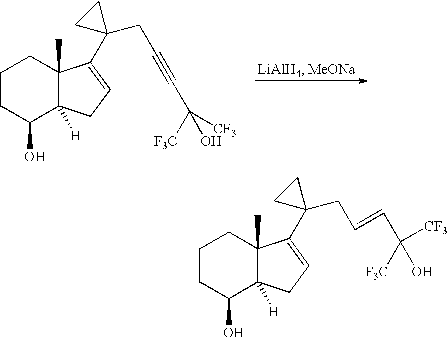

To a lithium aluminum hydride (4.5 mL, 4.5 mmol, 1.0M in THF) at 5° C. was added first solid sodium methoxide (245 mg, 4.6 mmol) and then dropwise solution of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (360 mg, 0.91 mmol) in tetrahydrofurane (5 mL). After addition was completed the mixture was stirred under reflux for 2.5 h. Then it was cooled in the ice-bath and quenched with water (2.0 mL) and sodium hydroxide (2.0 mL, 2.0 M water solution); diluted with ether (50 mL) stirred for 30 min, MgSO₄ (5 g) was than added and stirring was continued for 30 min. The residue after evaporation of the filtrates (0.42 g) was purified by FC (20 g, 20% AcOEt in hexane) to give the titled compound (315 mg, 0.79 mmol, 87%). [α]²⁸_{D}=+2.0 c 0.41, CHCl₃, ¹H NMR (CDCl₃): 6.24 (1H, dt, J=15.7, 6.7 Hz), 5.60 (1H, d, J=15.7 Hz), 5.38 (1H, br. s), 4.13 (1H, br. s), 3.27 (1H, s), 2.32-1.34 (12H, m), 1.15 (3H, s), 0.80-0.45 (4H, m); ¹³C NMR (CDCl₃): 155.89(0), 138.10(1), 126.21(1), 122.50(0, q, J=287 Hz), 119.15 (1), 76.09(0, sep. J=31 Hz), 69.57(1), 55.33(1), 47.30(0), 40.31(2), 36.05(2), 33.71(2), 30.10(2), 20.36(0), 19.46(3), 17.94(2), 11.96(2), 11.46(2); MS REI Calculated for Cl₉1H₂₄O₂F₆ M+ 398.1680. Observed M+ 398.1675.

### Example 32

### Synthesis of (3aR,7aR)-7a-Methyl-1-[1-(5,5,5-trifluoro-4-trifluoromethyl-4-trimethylsilanyloxy-pen-2E-enyl)-cyclopropyl]-3a,4,5,6,7,7a-hexahydro-3H-inden-4-one

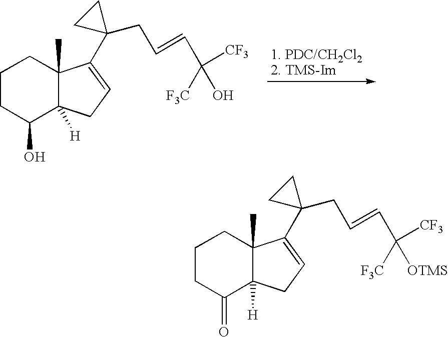

To a stirred suspension of (3aR,4S,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-ol (600 mg, 1.51 mmol) and Celite (2.0 g) in dichloromethane (10 mL) at room temperature wad added pyridinium dichromate (1.13 g, 3.0 mmol). The resulting mixture was stirred for 3.5 h filtered through silica gel (10 g), and then silica gel pad was washed with 25% AcOEt in hexane. The combined filtrate and washes were evaporated, to give a crude (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol, 92%). To a stirred solution of (3aR,7aR)-7a-Methyl-1-[1-(5,5,5-trifluoro-4-hydroxy-4-trifluoromethyl-pent-2E-enyl)-cyclopropyl]-3a,4,5,6,7,7a-hexahydro-3H-inden-4-one (550 mg, 1.39 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (623 mg, 1.33 mmol, 88%). [α]²⁸_{D}=−1.6 c 0.51, CHCl₃, ¹H NMR (CDCl₃): 6.14 (1H, dt, J=15.5, 6.7 Hz), 5.55 (1H, d, J=15.5 Hz), 5.35 (1H, m), 2.80 (1H, dd, J=10.7, 6.4 Hz), 2.47-1.74 (101H, m), 0.90 (3H, s), 0.76-0.40 (4H, m), 0.2 (9H, s); ¹³C NMR (CDCl₃): 210.99 (0), 154.28(0), 137.41(1), 126.26(1), 122.59(0, q, J=289 Hz), 120.89 (1), 64.31(1), 53.96(0), 40.60(2), 40.13(2), 35.00(2), 27.03(2), 24.21(2), 20.57(0), 18.53(3), 12.41(2), 10.79(2), 1.65 (3); MS HRES Calculated for C₂₂H₃₀O₂F₆Si M+H 469.1992. Observed M+H 469.1995.

### Example 33

### Synthesis of 1α,25-hydroxy-16-ene-20-cyclopropyl-23,24-E-ene-26,27-hexafluoro-19-nor-cholecalciferol (27)

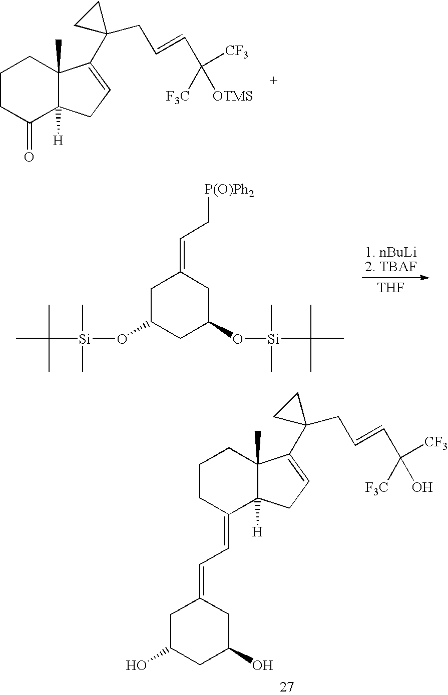

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (750 mg) after evaporation of the solvent was purified by FC (15 g, 5% AcOEt in hexane) to give a mixture of 1α,3β-Di(tert-Butyl-dimethyl-silanyloxy)-25-trimethylsilanyloxy-16-ene-20-cyclopropyl-23,24-E-ene-26,27-hexafluoro-19-nor-cholecalciferol and 1α,3β-Di(tert-Butyl-dimethyl-silanyloxy)-25-hydroxy-16-ene-20-cyclopropyl-23,24-E-ene-26,27-hexafluoro-19-nor-cholecalciferol (250 mg). To the mixture of 1α,3β-Di(tert-Butyl-dimethyl-silanyloxy)-25-trimethylsilanyloxy-6-ene-20-cyclopropyl-23,24-E-ene-26,27-hexafluoro-19-nor-cholecalciferol and 1α,3β-Di(tert-Butyl-dimethyl-silanyloxy)-25-hydroxy-16-ene-20-cyclopropyl-23,24-E-ene-26,27-hexafluoro-19-nor-cholecalciferol (250 mg) tetrabutylammonium fluoride (4 mL, 4 mmol, 1M solution in THF) was added, at room temperature. The mixture was stirred for 24 h. diluted with AcOEt (25 mL) and washed with water (5×20 mL), brine (20 mL) and dried over Na₂SO₄. The residue (270 mg) after evaporation of the solvent was purified by FC (10 g, 50% AcOEt in hexane and AcOEt) to give the titled compound (27) (157 mg, 0.30 mmol, 70%). [α]³_{D}=+63.3 c 0.45, EtOH. UV λmax (EtOH): 243 nm (ε30821). 251 nm (ε36064), 260 nm (ε2461 NMR (CDCl₃): 6.29 (1H, d, J=11.3 Hz), 6.24 (1H, dt, J=15.9, 6.4 Hz), 5.92 (1H, d, J=11.1 Hz), 5.61 (1H, d, J=15.7 Hz), 5.38 (1H, m), 4.13 (1H, m), 4.05 (1H, m), 2.88 (1H, s), 2.82-1.34 (19H, m), 0.770 (3H, s), 0.80-0.36 (4H, m); MS HRES Calculated for C₂₇H₃₄O₃F₆ M+H 521.2485. Observed M+H 521.2489.

### Example 34

### Synthesis of 1α,25-Dihydroxy-16-ene-20-cyclopropyl-23,24-E-ene-26,27-hexafluoro cholecalciferol (28)

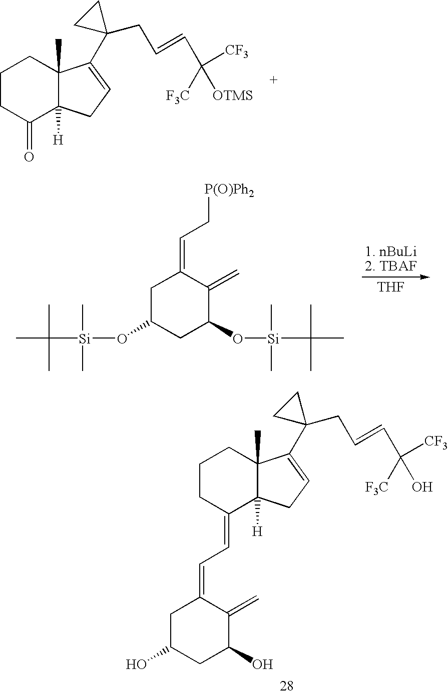

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2E-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with hexane (35 mL) washed brine (30 mL) and dried over Na₂SO₄. The residue (760 mg) after evaporation of the solvent was purified by FC (15 g, 10% AcOEt in hexane) to give a mixture of 1α,3β-Di(tert-Butyl-dimethyl-silanyloxy)-25-trimethylsilanyloxy-16-ene-20-cyclopropyl-23,24-E-ene-26,27-hexafluoro-cholecalciferol and 1α,3β-Di(tert-Butyl-dimethyl-silanyloxy)-25-hydroxy-16-ene-20-cyclopropyl-23,24-E-ene-26,27-hexafluoro-cholecalciferol (274 mg). To the mixture of 1α, 3β-Di(tert-Butyl-dimethyl-silanyloxy)-25-trimethylsilanyloxy-16-ene-20-cyclopropyl-23,24-E-ene-26,27-hexafluoro-cholecalciferol and 1α,3β-Di(tert-Butyl-dimethyl-silanyloxy)-25-hydroxy-16-ene-20-cyclopropyl-23,24-E-ene-26,27-hexafluoro-cholecalciferol (274 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (28) (167 mg, 0.31 mmol, 73%). [α]³_{D}=+18.3 c 0.41, EtOH. UV λmax (EtOH): 207 nm (ε17778), 264 nm (ε15767); ¹H NMR (CDCl₃): 6.36 (1H, d, J=11.1 Hz), 6.24 (1H, dt, J=15.7, 6.7 Hz), 6.07 (1H, d, J=11.3 Hz), 5.60 (1H, d, J=15.5 Hz), 5.35 (1H, m), 5.33 (1H, s), 5.00 (1H, s), 4.44 (1H, m), 4.23 (1H, m), 3.14 (1H, s), 2.80 (1H, m), 2.60 (1H, m), 2.40-1.40 (15H, m), 0.77 (3H, s), 0.80-0.36 (4H, m); MS HRES Calculated for C₂₈H₃₄O₃F₆ M+H 533.2485. Observed M+H 533.2483.

### Example 35

### Synthesis of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol

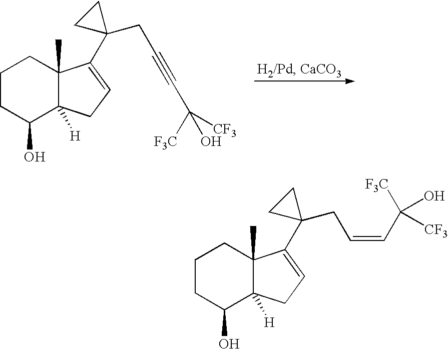

The mixture of (3aR,4S,7aR)-7a-Methyl-1-[1-(5,5,5-trifluoro-4-hydroxy-4-trifluoromethyl-pent-2-ynyl)-cyclopropyl]-3a,4,5,6,7,7a-hexahydro-3H-inden-4-ol (300 mg, 0.76 mmol), ethyl acetate (5 mL), hexane (12 mL), absolute ethanol (0.5 mL) quinoline (30 mL) and Lindlar catalyst (75 mg, 5% Pd on CaCO₃) was hydrogenated at room temperature for 2 h. The reaction mixture was filtered through a celite pad and the pad was washed with AcOEt. The solvent was evaporated to give the titled compound (257 mg, 0.65 mmol, 87%). [α]²⁸_{D}=+1.8 c 0.61, CHCl₃, ¹H NMR (CDCl₃): 6.08 (1H, dt, J=12.3, 6.7 Hz), 5.47 (1H, m), 5.39 (1H, d, J=12.1 Hz), 4.15 (1H, br. s), 3.28 (1H, s), 2.52-1.34 (12H, m), 1.16 (3H, s), 0.78-0.36 (4H, m); ¹³C NMR (CDCl₃): 156.66(0), 141.77(1), 126.51(1), 122.79(0, q, J=285 Hz), 115.77 (1), 69.59(1), 55.41(1), 47.28(0), 36.44(2), 35.90 (2), 33.75(2), 30.22(2), 20.89(0), 19.41 (3), 17.94(2), 12.05 (2), 11.11 (2); MS HRES Calculated for C₁₉H₂₄O₂F₆ M+H 399.1753. Observed M+H 399.1757.

### Example 36

### Synthesis of (3aR,7aR)-7a-Methyl-1-[1-(5,5,5-trifluoro-4-trifluoromethyl-4-trimethylsilanyloxy-pen-2Z-enyl)-cyclopropyl]-3a,4,5,6,7,7a-hexahydro-3H-inden-4-one

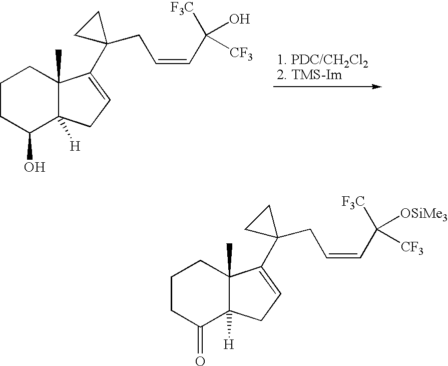

To a stirred suspension of (3aR,4S,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-ol (617 mg, 1.55 mmol) and Celite (2.0 g) in dichloromethane (10 mL) at room temperature wad added pyridinium dichromate (1.17 g, 3.1 mmol). The resulting mixture was stirred for 2.5 h filtered through silica gel (5 g), and then silica gel pad was washed with 20% AcOEt in hexane. The combined filtrate and washes were evaporated, to give a crude (3aR,7aR)-7a-Methyl-1-[1-(5,5,5-trifluoro-4-hydroxy-4-trifluoromethyl-pentenyl)-cyclopropyl]-3a,4,5,6,7,7a-hexahydro-3H-inden-4-one (600 mg, 1.51 mmol, 98%). To a stirred solution of (3aR,7aR)-7a-Methyl-1-[1-(5,5,5-trifluoro-4-hydroxy-4-trifluoromethyl-pent-2Z-enyl)-cyclopropyl]-3a,4,5,6,7,7a-hexahydro-3H-inden-4-one (600 mg, 1.51 mmol) in dichloromethane (15 mL) at room temperature was added trimethylsilyl-imidazole (1.76 mL, 12.0 mmol). The resulting mixture was stirred for 1.0 h filtered through silica gel (10 g) and the silica gel pad was washed with 10% AcOEt in hexane. Combined filtered and washes were evaporated to give the titled compound (640 mg, 1.37 mmol, 88%). [α]²⁸_{D}=−0.2 c 0.55, CHCl₃. ¹H NMR (CDCl₃): 5.97 (1H, dt, J=12.2, 6.2 Hz), 5.40 (1H, m), 5.38 (1H, d, J=12.2 Hz), 2.82 (1H, dd, J=10.7, 6.6 Hz), 2.60-1.74 (10H, m), 0.89 (3H, s), 0.75-0.36 (4H, m), 0.21 (9H, s); ¹³C NMR (CDCl₃): 210.56 (0), 154.30(0), 139.28(1), 125.81(1), 122.52(0, q, J=289 Hz), 118.17 (1), 64.11(1), 53.69(0), 40.43(2), 35.51(2), 34.85(2), 26.94(2), 24.07(2), 20.89(0), 18.39(3), 12.26(2), 10.61(2), 1.43 (3); MS HRES Calculated for C₂₂H₃₀O₂F₆Si M+H 469.1992. Observed M+H 469.1992.

### Example 37

### Synthesis of 1α,25-Dihydroxy-16-ene-20-cyclopropyl-23,24-f-ene-2627-hexafluoro-19-nor-cholecalciferol (29)

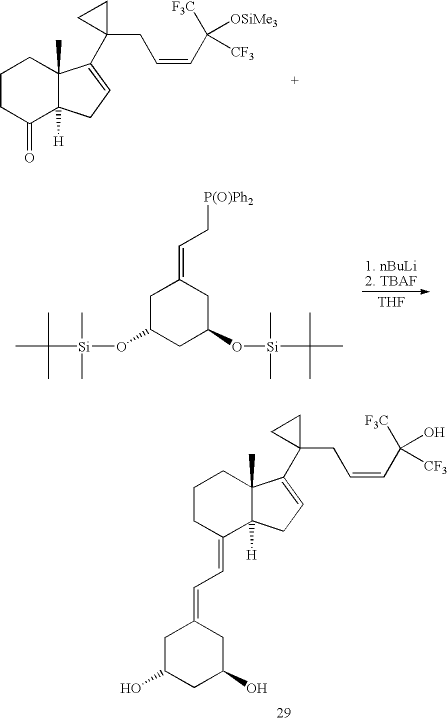

To a stirred solution of a (1R,3R)-1,3-bis-((tert-butyldimethyl)silanyloxy)-5-[2-(diphenylphosphinoyl)ethylidene]-cyclohexane (514 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-trimethylsilanyloxy-pent-2Z-enyl)-cyclopropyl]-3a,4,5,6,7,7a-hexahydro-3H-inden-4-one (194 mg, 0.41 mmol, in tetrahydrofurane (2 mL) was added dropwise. The reaction mixture was stirred at −72° C. for 3.0 h diluted with hexane (35 mL) washed brine (30 μL) and dried over Na₂SO₄. The residue (750 mg) after evaporation of the solvent was purified by FC (15 g, 10% AcOEt in hexane) to give a mixture of 1α,3β-Di(tert-Butyl-dimethyl-silanyloxy)-25-trimethylsilanyloxy-16-ene-20-cyclopropyl-23,24-Z-ene-26,27-hexafluoro-19-nor-cholecalciferol and 1α,3β-Di(tert-Butyl-dimethyl-silanyloxy)-25-hydroxy-16-ene-20-cyclopropyl-23,24-Z-ene-26,27-hexafluoro-19-nor-cholecalciferol (230 mg). To the mixture of 1α,3β-Di(tert-Butyl-dimethyl-silanyloxy)-25-trimethylsilinyloxy-16-ene-20-cyclopropyl-23,24-Z-ene-26,27-hexafluoro-19-nor-cholecalciferol and 1α,3β-Di(tert-Butyl-dimethyl-silanyloxy)-25-hydroxy-16-ene-20-cyclopropyl-23,24-Z-ene-26,27-hexafluoro-19-nor-cholecalciferol (230 mg) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (260 mg) after evaporation of the solvent was purified by FC (10 g, 50% AcOEt in hexane and AcOEt) to give the titled compound (29) (1327 mg, 0.25 mmol, 62%). [α]²⁸_{D}=+53.6 c 0.33, EtOH. UV λmax (EtOH): 243 nm (ε26982 251 nm (ε32081), 260 nm (ε21689); ¹H NMR (CDCl₃): 6.29 (1H, d, J=10.7 Hz), 6.08 (1H, dt, J=12.5, 6.7 Hz), 5.93 (1H, d, J=11.1 Hz), 5.46 (1H, m), 5.40 (1H, d, J=12.7 Hz)), 4.12 (1H, m), 4.05 (1H, m), 3.14 (1H, s), 2.80-1.40 (19H, n), 0.77 (3H, s), 0.80-0.36 (4H, m); MS HRES Calculated for C₂₇H₃₄O₃F₆ M+H 521.2485. Observed M+H 521.2487.

### Example 38

### Synthesis of 1α,25-Dihydroxy-16-ene-20-cyclopropyl-23,24-Z-ene-26,27-hexafluoro-cholecalciferol (30)

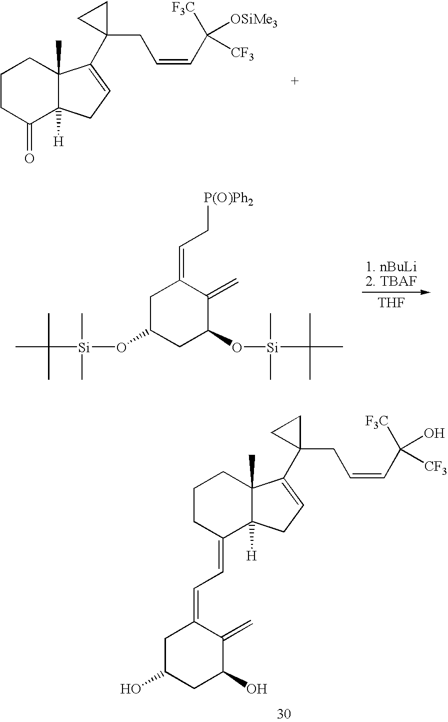

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (525 mg, 0.90 mmol) in tetrahydrofurane (6 mL) at −78° C. was added n-BuLi (0.57 mL, 0.91 mmol). The resulting mixture was stirred for 15 min and solution of (3aR,7aR)-7a-Methyl-1-[1-(5,5,5-trifluoro-4-trifluoromethyl-4-methylsilanyloxy-pent-2Z-enyl)-cyclopropyl]-3a,4,5,6,7,7a-hexahydro-3H-inden-4-one (200 mg, 0.43 mmol, in tetrahydrofurane (2 mL) was added dropwise. The reaction mixture was stirred at −72° C. for 2.5 h diluted with, hexane (35 mL) washed brine (30 mL) and dried over Na₂SO₄. The residue (680 mg) after evaporation of the solvent was purified by FC (15 g, 10% AcOEt in hexane) to give a mixture of 1α,3β-Di(tert-Butyl-dimethyl-silanyloxy)-25-trimethylsilanyloxy-16-ene-20-cyclopropyl-23,24-Z-ene-26,27-hexafluoro-cholecalciferol and 1α,3β-Di(tert-Butyl-dimethyl-silanyloxy)-25-hydroxy-16-ene-20-cyclopropyl-23,24-Z-ene-26,27-hexafluoro-cholecalciferol (310 mg). To the mixture of 1α,3β-Di(tert-Butyl-dimethyl-silanyloxy)-25-trimethylsilanyloxy-16-ene-20-cyclopropyl-23,24-Z-ene-26,27-hexafluoro-cholecalciferol and 1α,3β-Di(tert-Butyl-dimethyl-silanyloxy)-25-hydroxy-16-ene-20-cyclopropyl-23,24-Z-ene-26,27-hexafluoro-cholecalciferol (310 mg) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (370 mg) after evaporation of the solvent was purified by FC (10 g, 50% AcOEt in hexane and AcOEt) to give the titled compound (30) (195 mg, 0.37 mmol, 85%). [α]³⁰_{D}=+9.4 c 0.49, EtOH. UV λmax (EtOH): 262 nm (ε11846); ¹H NMR (CDCl₃): 6.36 (1H, d, J=11.1 Hz), 6.08 (2H, m), 5.44 (1H, m), 5.40 (1H, d, J=12.3 Hz), 5.32 (1H, s), 5.00 (11H, s), 4.43 (1H, m), 4.23 (1H, m), 3.08 (1H, s), 2.80 (1H, m), 2.60 (1H, m), 2.55-1.40 (15H, m), 0.77 (3H, s), 0.80-0.34 (4H, m); MS HRES Calculated for C₂₈H₃₄O₃F₆ M+H 533.2485. Observed M+H 533.2502.

### Example 39

### Synthesis of 1α,25-Dihydroxy-16-ene-20-cyclopropyl-19-nor-cholecalciferol (31)

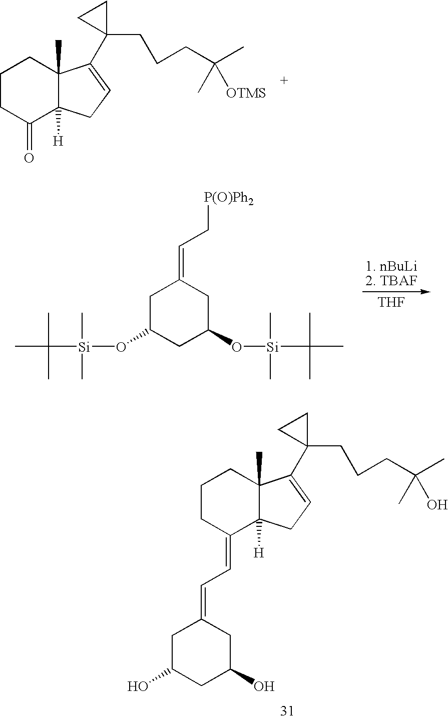

To a stirred solution of a (1R,3R)-1,3-bis-((tert-butyldimethyl)silanyloxy)-5-[2-(diphenylphosphinoyl)ethylidene]-cyclohexane (697 mg, 1.22 mmol) in tetrahydrofurane (9 mL) at −78° C. was added n-BuLi (0.77 mL, 1.23 mmol). The resulting mixture was stirred for 15 min and solution of (3aR,7aR)-7a-Methyl-1-[1-(4-methyl-4-trimethylsilanyloxy-pentyl)-cyclopropyl]-3a,4,5,6,7,7a-hexahydro-3H-inden-4-one (220 mg, 0.61 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (900 mg) after evaporation of the solvent was purified by FC (15 g, 10% AcOEt in hexane) to give 1α,3β-Di(tert-Butyl-dimethyl-silanyloxy)-25-trimethylsilanyloxy-16-ene-20-cyclopropyl-19-nor-cholecalciferol (421 mg, 0.59 mmol). To the 1α,3β-Di(tert-Butyl-dimethyl-silanyloxy)-25-trimethylsilanyloxy-16-ene-20-cyclopropyl-26,27-hexadeutero-19-nor-cholecalciferol (421 mg, 0.59 mmol) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (450 mg) after evaporation of the solvent was purified by FC (15 g, 50% AcOEt in hexane and AcOEt) to give the titled compound (31) (225 mg, 0.54 mmol, 89%). [α]²⁹_{D}=+69.5 c 0.37, EtOH. UV λmax (EtOH): 243 nm (ε27946 251 nm (ε33039), 261 nm (ε22701); ¹H NMR (CDCl₃): 6.30 (1H, d, J=11.3 Hz), 5.93 (1H, d, J=11.3 Hz), 5.36 (1H, m), 4.12 (1H, m), 4.04 (1H, m), 2.75 (2H, m), 2.52-1.04 (22H, m), 1.18 (6H, s), 0.79 (3H, s), 0.65-0.26 (4H, m); ¹³C NMR (CDCl₃): 157.16(0), 142.33(0), 131.25(0), 124.73(1), 123.76(1), 115.50(1), 71.10(0), 67.39(1), 67.19(1), 59.47(1), 50.12(0), 44.60(2), 43.84(2), 42.15(2), 38.12(2), 37.18(2), 35.57(2), 29.26(3), 29.11(2), 29.08(3), 28.48(2), 23.46(2), 22.26(2), 21.27(0), 17.94(3), 12.70(2), 10.27(2); MS HRES Calculated for C₂₇H₄₂O₃ M+H 415.3207. Observed M+H 415.3207.

### Example 40

### Synthesis of 1α,25-Dihydroxy-16-ene-20-cyclopropyl-cholecalciferol (32)

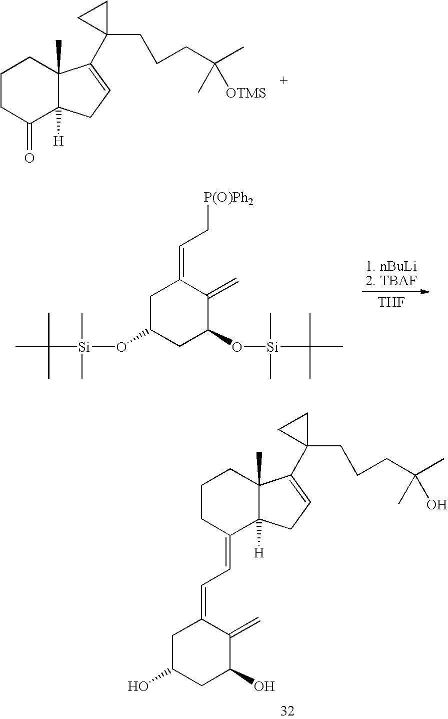

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (675 mg, 1.16 mmol) in tetrahydrofurane (8 mL) at −78° C. was added n-BuLi (0.73 mL, 1.17 mmol). The resulting mixture was stirred for 15 min and solution of (3aR,7aR)-7a-Methyl-1-[1-(4-methyl-4-trimethylsilanyloxy-pentyl)-cyclopropyl-3a,4,5,6,7,7a-hexahydro-3H-inden-4-one (210 mg, 0.58 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (850 mg) after evaporation of the solvent was purified by FC (15 g, 10% AcOEt in hexane) to give 1α,3β1-Di(tert-Butyl-dimethyl-silanyloxy)-25-trimethylsilanyloxy-16-ene-20-cyclopropyl-cholecalciferol (382 mg, 0.53 mmol). To the 1 (,3-Di(tert-Butyl-dimethyl-silanyloxy)-25-trimethylsilanyloxy-16-ene-20-cyclopropyl-cholecalciferol (382 mg, 0.53 mmol) tetrabutylammonium fluoride (4 mL, 4 mmol, 1M solution in THF) was added, at room temperature. The mixture was stirred for 15 h. diluted with AcOEt (25 mL) and washed with water (5×20 ml), brine (20 mL) and dried over Na₂SO₄. The residue (380 mg) after evaporation of the solvent was purified by FC (15 g, 50% AcOEt in hexane and AcOEt) to give the titled compound (32) (204 mg, 0.48 mmol, 83%). [α]²⁹_{D}=+16.1 c 0.36, EtOH. UV λmax (EtOH): 208 nm (ε17024), 264 nm (ε16028); ¹H NMR (CDCl₃): 6.37 (1H, d, J=11.3 Hz), 6.09 (1H, d, J=11.1 Hz), 5.33 (2H, m), 5.01 (1H, s), 4.44 (1H, m), 4.23 (11H, m), 2.80 (11H, m), 2.60 (1H, m), 2.38-1.08 (20H, m), 1.19 (6H, s), 0.79 (3H, s), 0.66-0.24 (4H, m); ¹³C NMR (CDCl₃): 157.07(0), 147.62(0), 142.49(0), 133.00(0), 124.90(1), 124.73(1), 117.19(1), 111.64(2), 71.10(1), 70.70(0), 66.88(1), 59.53(1), 50.28(0), 45.19(2), 43.85(2), 42.86(2), 38.13(2), 35.59(2), 29.27(2), 29.14(3), 28.65(2), 23.57(2), 22.62(2), 21.29(0), 17.84(3), 12.74(2), 10.30(2); MS HRES Calculated for C₂₈L₂O₃ M+Na 449.3026. Observed M+Na 449.3023.

### Example 41

### Synthesis of 1,25-Dihydroxy-21-(2R,3-dihydroxy-3-methyl-butyl)-20R-Cholecalciferol (33)

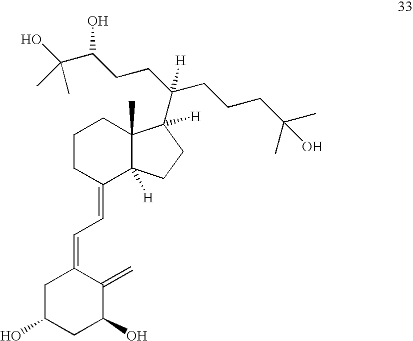

[1R,3aR,4S,7]-2(R)-[4-(1,1-dimethylethyl)dimethyl-silanyloxy)-7a-methyl-octahydro-inden-1-yl]-6-methyl-heptane-1,6-diol (34) and [1R,3aR,4S,7aR]-2(S)-[4-(1,1-dimethylethyl)dimethyl-silanyloxy)-7a-methyl-octahydro-inden-1-yl]-6-methyl-heptane-1,6-diol (35)

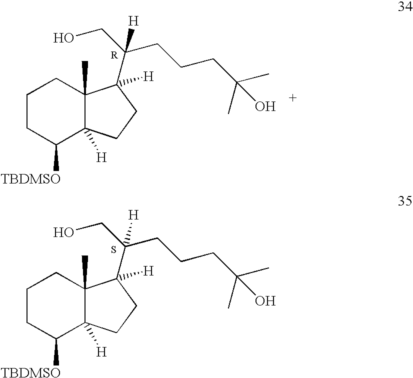

A solution of the alkenol in tetrahydrofuran (9 mL) was cooled in an ice bath and a 1 M solution of borane-THF in tetrahydrofuran (17 mL) was added dropwise in an originally effervescent reaction. The solution was stirred overnight at room temperature, re-cooled in an ice bath water (17 mL) was added dropwise followed by sodium percarbonate (7.10 g, 68 mmol). The mixture was immersed into a 50° C. bath and stirred for 70 min to generate a solution. The two-phase system was allowed to cool then equilibrated with 1:1 ethyl acetate-hexane (170 mL). The organic layer was washed with water (2×25 mL) then brine (20 mL), dried and evaporated to leave a colorless oil (2.76 g). This material was passed through a short flash column using 1:1 ethyl acetate-hexane and silica gel G. The effluent, obtained after exhaustive elution, was evaporated, taken up in ethyl acetate, filtered and chromatographed on the 2×18″ 15-20 u silica YMC HPLC column using 2:1 ethyl acetate-hexane as mobile phase and running at 100 mL/min. Isomer 34 emerged at an effluent maximum of 2.9 L, colorless oil, 1.3114 g, [α]_{D}+45.2° (methanol, c 0.58; ¹H NMR δ −0.002 (3H, s), 0.011 (3H, s), 0.89 (9H, s), 0.93 (3H, s), 1.17 (1H, m), 1.22 (6H, s), 1.25-1.6 (16H, m), 1.68 (1H, m), 1.80 (2H, m), 1.89 (1H, m), 3.66 (1H, dd, J=4.8 and 11 Hz), 3.72 (1H, dd, J=3.3 and 11 Hz), 4.00 (1H, m); LR-ES(−) m/z 412 (M), 411 (M−H); HR-ES(+): calcd for (M+Na) 435.3265, found: 435.3269.

Isomer 35 at was eluted at an effluent maximum of 4.9 L, colorless oil, 0.8562 g that crystallized upon prolonged standing: mp 102-3°, [α]_{D}+25.2° (methanol, c 0.49); ¹H NMR δ −0.005 (3H, s), 0.009 (3H, s), 0.89 (9H, s), 0.93 (3H, s), 1.16 (11H, m), 1.22 (6H, s), 1.3-1.5, (14H, m), 1.57 (2H, m), 1.67 (1H, m), 1.80 (2H, m), 1.91 (1H, m), 3.54 (1H, dd, J=4.8 and 11 Hz), 3.72 (1H, dd, J=2.9 and 11 Hz), 4.00 (1H, m); LR-ES(−) m/z 412 (M), 411 (M−H). Anal. Calcd for C₂₄H₄₈O₃Si: C, 69.84, H, 11.72; found: C, 69.91; H, 11.76.

### [1R,3aR,4S,7R]-6(R)-[4-(tert-Butyl-dimethyl-silanyloxy)-7a-methyl-octahydro-inden-1-yl]-7-iodo-2-methyl-heptan-2-ol (36)

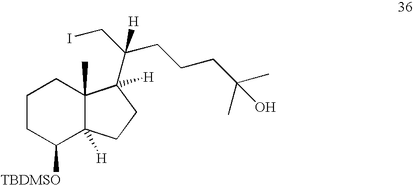

A stirred mixture of triphenylphosphine (0.333 g, 1.27 mmol) and imidazole (0.255 g, 3 mmol) in dichloromethane (3 mL) was cooled in an ice bath and iodine (0.305 g, 1.20 mmol) was added. This mixture was stirred for 10 min then a solution of 34 (0.4537 g, 1.10 mmol) in dichloromethane (3 mL) was added dropwise over a 10 min period. The mixture was stirred in the ice bath for 30 min then at ambient temperature for 2¾ h. TLC (1:1 ethyl acetate-hexane) confirmed absence of educt. A solution of sodium thiosulfate (0.1 g) in water (5 mL) was added, the mixture equilibrated and the organic phase washed with 0.1 N sulfuric acid (1.0 mL) containing a few drops of brine then with 1:1 water-brine (2×10 mL), once with brine (10 mL) then dried and evaporated. The residue was purified by flash chromatography using 1:9 ethyl acetate-hexane as mobile phase to furnish 36 as a colorless syrup, 0.5637 g, 98%: ¹H NMR δ −0.005 (3H, s), 0.010 (3H, s), 0.89 (9H, s), 0.92 (3H, s), 1.23 (6H, s), 1.1-1.6 (16H, m), 1.68 (1H, m), 1.79 (2H, m), 1.84 (1H, m), 3.37 (1H, dd, J=4 and 10 Hz), 3.47 (1H, dd, J=3 and 10 Hz), 4.00 (1H, m); LR-EI(+) m/z 522 (M), 465 (M-C₄H₉), 477 (M-C₄H₉—H₂O); HR-EI(+): calcd for C₂₄H₄₇IO₂Si: 522.2390, found: 522.2394.

### [1R,3aR,4S,7aR]-6(S)-[4-tert-Butyl-dimethyl-silanyloxy)-7a-methyl-octahydro-inden-1-yl]-2-methyl-non-8-yn-2-ol (37)

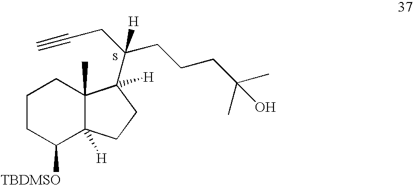

Lithium acetylide DMA complex (0.110 g, 1.19 mmol) was added to a solution of 36 (0.2018 g (0.386 mmol) in dimethyl sulfoxide (1.5 mL) and tetrahydrofuran (0.15 mL). The mixture was stirred overnight. TLC (1:4 ethyl acetate-hexane) showed a mixture of two spots traveling very close together (Rf 0.52 and 0.46). Fractions at the beginning of the eluted band contained pure alkenol, which is the elimination product of 36, and was produced as the major product. Fractions at the end of the elution band, however, were also homogeneous and gave the desired acetylene 37 upon evaporation. The NMR spectra of 37 and its 6-epimer which served for identification were previously reported.

### [1R,3aR,4S,7aR]-7-Benzenesulfonyl-6(S)-[4-(tert-butyl-dimethyl-silanyloxy)-7a-methyl-octahydro-inden-1-yl]-2-methyl-heptan-2-ol (38)

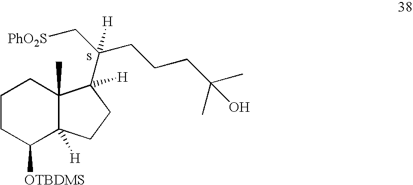

A mixture of 37b (0.94 g, 1.8 mmol), sodium benzenesulfinate (2.18 g, 13 mmol) and N,N-dimethylformamide (31.8 g) was stirred at room temperature for 12 h, then in a 40° C. bath for ca.6 h until all educt was converted as shown by TLC (1:4 ethyl acetate-hexane). The solution was equilibrated with 1:1 ethyl acetate-hexane (120 mL) and 1:1 brine-water (45 mL). The organic layer was washed with water (4×25 mL) brine (10 mL), then dried and evaporated to leave a colorless oil, 1.0317 g. This material was flash-chromatographed using a stepwise gradient (1:9, 1:6, 1:3 ethyl acetate-hexane) to give a colorless oil, 0.930 g, 96%:300 MHz ¹H NMR δ −0.02 (3H, s), 0.00 (3H, s), 0.87 (9H, s), 0.88 (3H, s), 1.12 (1H, m), 1.20 (6H, s), 1.2-1.8 (18H, m), 1.81 (1H, m), 3.09 (2H, m), 3.97 (1H, brs), 7.59 (3H, m), 7.91 2H, m).

### [1R,3aR,4S,7aR]-1-(1(S)-Benzenesulfonylmethyl-5-methyl-5-trimethylsilanyloxy-hexyl)-4-(tert-butyl-dimethyl-silanyloxy)-7a-methyl-octahydro-indene (39)

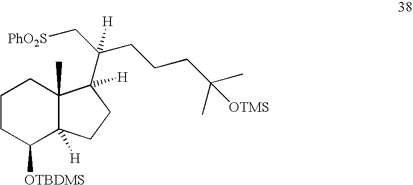

1-(Trimethylsilyl)imidazole (1 mL) was added to a solution of 38 (0.8 g) in cyclohexane (10 mL) and stirred overnight then flash-chromatographed using a stepwise gradient of hexane, 1:39 and 1:19 ethyl acetate-hexane. The elution was monitored by TLC (1:4 ethyl acetate-hexane) leading to 39 as a colorless syrup, 0.7915 g: 300 MHz ¹H NMR 60.00 (31H, s), 0.02 (3H, s), 0.12 (9H, s), 0.90 (12H, s, t-butyl+7a-Me), 1.16 (1H, m), 1.20 (6H, s), 1.2-1.6 (15H, m), 1.66-1.86 (3H, m), 3.1.0 (2H, m), 4.00 (1H, brs), 7.56-7.70 (3H, m), 7.93 (2H, m).

### [1R,3aR,4S,7aR]-6(R)-[4-(tert-Butyl-dimethyl-silanyloxy)-7a-methyl-octahydro-inden-1-yl]-2,10-dimethyl-undecane-2,3(R),10-triol (40)

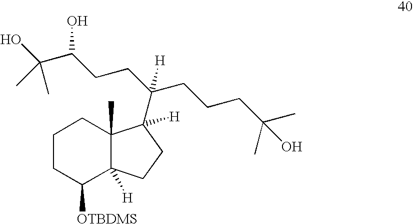

A solution of 39 (0.7513 g, 1.23 mmol) and diol (0.508 g, 1.85 mmol) in tetrahydrofuran (28 mL) was cooled to −35° C. then 2.5 M butyllithium in hexane (2.75 mL) was added dropwise. The temperature was allowed to rise to −20° C. and maintained at that temperature for 6 h or until the educt was consumed. Reaction progress was monitored by TLC (1:4 ethyl acetate-hexane) exhibiting the educt (Rf 0.71) and the two epimeric diols (Rf 0.09 and 0.12). Toward the end of the reaction period the temperature was increased briefly to 0° C., lowered again to −10, then saturated ammonium chloride (25 mL) was added followed by ethyl acetate (50 mL) and enough water to dissolve the precipitated salts. The resulting aqueous phase was extracted with ethyl acetate (15 mL). The combined extracts were washed with brine (15 mL), dried and evaporated. The resulting syrup was flash-chromatographed using a stepwise gradient of 1:9, 1:6, 1:4 and 1:1 ethyl acetate-hexane to give 39a as a colorless syrup, 0.8586 g. This material was dissolved in a mixture of tetrahydrofuran (30 mL) and methanol (18 mL), then 5% sodium amalgam (20 g) was added. The reductive de-sulfonylation was complete after stirring of the mixture for 14 h. Progress of the reaction was monitored by TLC (1:1 ethyl acetate-hexane) which showed the disappearance of the epimeric diols (Rf 0.63 and 0.74) and the generation of 40a (Rf 0.79) and the partially de-silylated analogue 40 (Rf 0.16). The mixture was diluted with methanol (20 mL), stirred for 3 min, then ice (20 g) was added, stirred for 2 min and the supernatant decanted into a mixture containing saturated ammonium chloride (50 mL). The residue was repeatedly washed with small amounts of tetrahydrofuran that was also added to the salt solution, which was then equilibrated with ethyl acetate (80 mL). The aqueous layer was re-extracted once with ethyl acetate (20 mL), the combined extracts were washed with brine (10 mL) then dried and evaporated. The resulting colorless oil containing both 40a and 40 was dissolved in 10 mL of a 1 N oxalic acid solution in methanol (prepared from the dihydrate) effecting the selective hydrolysis of the trimethylsilyl ether within minutes. Calcium carbonate (1 g) was added and the suspension stirred overnight, then filtered. The solution was evaporated and the resulting residue flash-chromatographed using a stepwise gradient of 1:4, 1:2, 1:1 and 2:1 ethyl acetate-hexane giving a residue of the triol 40 that crystallized in very fine branching needles from acetonitrile, 0.45 g: mp 94-95° C., [α]_{D}+44.10 (methanol, c 0.37); 400 MHz ¹H NMR δ −0.005 (3H, s), 0.007 (3H, s), 0.89 (9H, s), 0.92 (3H, s), 1.15 (1H, m), 1.16 (3H, s), 1.21 (9H, s), 1.2-1.6 (19H, m), 1.67 (1H, m), 1.79 (2H, m), 1.90 (21H, m), 2.06 (1H, m), 3.31 (1H, brd, J=10 Hz), 4.00 (1H, brs), LR-ES(−) m/z: 533 (M+Cl), 497 (N-4-H); HR-ES(+): Calcd for C₂₉H₅₈O₄Si+Na: 521.3996, found: 521.4003. Anal Calcd for C₂₉H₅₈O₄Si: C, 69.82, H, 11.72; found: C, 69.97; H, 11.65.

### [1R,3aR,4S,7aR]-6(R)-(4-Hydroxy-7a-methyl-octahydro-inden-1-yl)-2,10-dimethyl-undecane-2,3(R),10-triol (41)

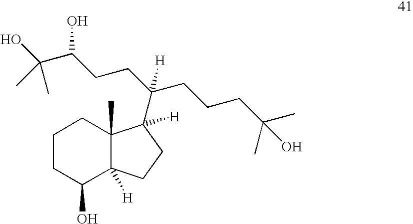

A stirred solution of the triol 40 (0.4626 g, 0.927 mmol) in acetonitrile (10 mL) and dioxane (0.7 mL) was cooled to 10° C. and a fluorosilicic acid solution (2 mL) was added dropwise. The cooling bath was removed, the 2-phase system further diluted with acetonitrile (2 mL) then stirred at room temperature for 3¼ h. The disappearance of educt was monitored by TLC (ethyl acetate). The mixture was equilibrated with water (10 mL) and ethyl acetate (30 mL). The aqueous phase was re-extracted with ethyl acetate (2×20 mL), the combined extracts were washed with water (5 mL) and brine (10 mL), then 1:1 brine-saturated sodium hydrogen carbonate solution and dried. The residue was purified by flash-chromatography using a step-wise gradient from 1:1 to 2:1 ethyl acetate-hexane and neat ethyl acetate to give a residue that was taken up in 1:1 dichloromethane-hexane, filtered and evaporated to furnish amorphous solids, 0.3039 g (85%): [α]_{D}+42.6° (methanol, c 0.48); ¹H NMR (DMSO-d₆): δ 0.87 (3H, s), 0.97 (3H, s), 1.02 (3H, s), 1.04 (6H, s), 1.1-1.4 (18H, m), 1.5-1.8 (4H, m), 1.84 (1H, m), 2.99 (1H, dd, J=6 and 10 Hz), 3.87 (1H, brs), 4.02 (1H, s, OH), 4.05 (1H, s, OH), 4.16 (11, d, OH, J=3.6 Hz), 4.20 (1H, d, OH, J=6.4 Hz); LR-ES(+): m/z 384 (M), 383 (M−H); HR-ES(+): Calcd for (M+Na) 407.3132, found: 407.3134.

### [1R,3aR,4S,7aR]-1-{5-Hydroxy-5-methyl-1(R)-[2-(2,2,5,5-tetramethyl-[1,3]dioxolan-4(R)-yl)-ethyl]-hexyl}-7a-methyl-octahydro-4-inden-4-ol (42)

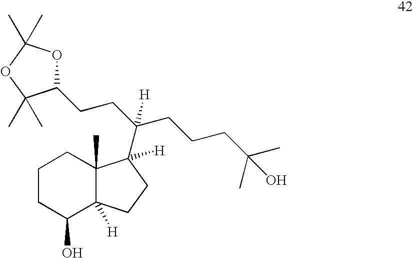

A solution of the tetrazol 40 (0.2966 g, 0.771 mmol) and pyridinium tosylate (100 mg) in acetone (8 mL) and 2,2-dimethoxypropane (8 mL) was kept at room temperature for 12 h. TLC analysis (ethyl acetate) showed the absence of educt (Rf 0.21) and two new spots with Rf 0.82 and 0.71, the former the expected 42 and the latter assumed to be the methylacetal. The reaction mixture was diluted with water (5 mL) and stirred for 10 min. At that time only the spot with higher Rf value was observed. The mixture was neutralized with sodium hydrogen carbonate (0.5 g) then equilibrated with ethyl acetate (50 mL) and brine (5 mL). The organic layer was washed with water (5 mL) and brine (5 mL) then dried and evaporated to leave a sticky residue (0.324 g) that was used directly in the next step: 300 MHz ¹H NMR: δ 0.94 (3H, s), 1.10 (3H, s), 1.20 (1H, m), 1.22 (6H, s), 1.25 (3H, s), 1.34 (3H, s), 1.41 (3H, s), 1.2-1.65 (20H, m), 1.78-1.86 (3H, m), 1.93 (1H, m), 3.62 (1H, dd, J=4.6 and 8.3 Hz), 4.08 (1H, brs).

### [1R,3aR,4S,7aR]-Acetic acid 1-{5-hydroxy-5-methyl-[(R)-[2-(2,2,5,5-tetramethyl-[1,3]dioxolan-4(R)-yl)-ethyl]-hexyl}-7a-methyl-octahydro-inden-4-yl ester (43)

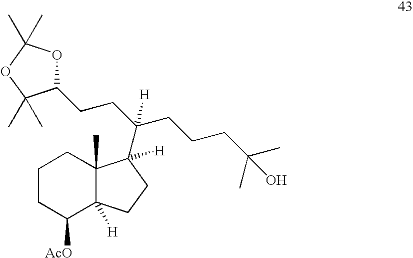

The residue obtained above was dissolved in pyridine (6.9 g) and further diluted with acetic anhydride (3.41 g). The mixture was allowed to stand at room temperature for 24 h, then in a 35° C. bath for ca. 10 h until the educt was no longer detectable (TLC, ethyl acetate). The mixture was diluted with toluene and evaporated. The residue was purified by flash chromatography (1:4 ethyl acetate-hexane) to give 43 as colorless syrup, 0.3452 g, 97%: ¹H NMR: δ 0.89 (3H, s), 1.10 (3H, s), 1.20 (1H, m), 1.22 (6H, s), 1.25 (33H, s), 1.33 (3H, s), 1.41 (3H, s), 1.25-1.6 (199H, m), 1.72 (1H, m), 1.82 (2H, m), 1.95 (1H, m), 2.05 (3H, s), 3.63 (1H, dd, J=4.4 and 8.4 Hz), 5.15 (1H, brs); LR-FAB(+) m/z 467 (+H), 465 (M−H), 451 (M-Me).

### [1R,3aR,4S,7aR]-Acetic acid 1-[4(R),5-dihydroxy-[(R)-(4-hydroxy-4-methyl-pentyl)-5-methyl-hexyl]-7a-methyl-octahydro-inden-4-yl ester (44)

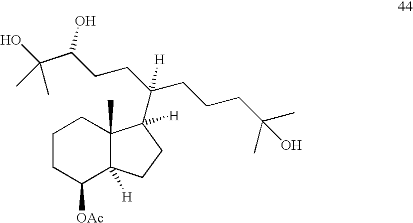

A solution of 43 (0.334 g, 0.716 mmol) in 80% acetic acid (2 mL) was kept in a 68° C. bath. TLC (ethyl acetate, Rf 0.33) monitored the progress of the hydrolysis. The educt was no longer detectable after 2.5 h. The mixture was evaporated then co-evaporated with a small amount of toluene to leave a colorless film (0.303 g) that was used directly in the next step: 300 MHz 1H NMR: δ 0.89 (3H, s), 1.17 (3H, s), 1.22 (6H, s), 1.56 (3H, s), 1.1-1.6 (21H, m), 1.6-2.0 (5H, m), 2.04 (3H, s), 3.32 (1H, brd, J=10 Hz), 5.15 (1H, brs).

### [1R,3aR,4S,7aR]-Acetic acid 1-[4(R)-[dimethyl-(1,1,2-trimethyl-propyl)-silanyloxy]-5-hydroxy-1(R)-(4-hydroxy-4-methyl-pentyl)-5-methyl-hexyl]-7a-methyl-octahydro-inden-4-yl ester (45)

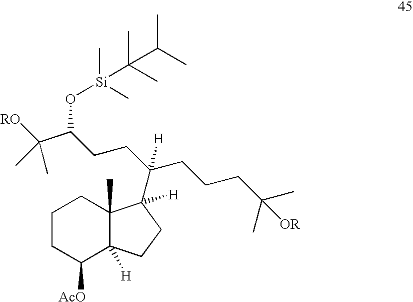

A solution of the triol 44 (0.30 g), imidazole (0.68 g, 10 mmol) and dimethylthexylsilyl chloride (1.34 g, 7.5 mmol) in N,N-dimethylformamide (6 g) was kept at room temperature. After 48 h 4-(N,N-dimethylamino)pyridine (15 mg) was added and the mixture stirred for an additional 24 h. Reaction progress was monitored by TLC (ethyl acetate; 24, Rf 0.83; 25a, Rf 0.38). The mixture was diluted with water (2 mL), stirred for 10 min then distributed between ethyl acetate (45 mL) and water (20 mL). The aqueous layer was extracted once with ethyl acetate (10 mL). The combined organic phases were washed with water (4×12 mL) and brine (8 mL) then dried and evaporated. The residual oil was purified by flash-chromatography using a stepwise gradient of 1:9 and 1:4 ethyl acetate-hexane to give 45 as colorless syrup. A small amount of unreacted educt (80 mg) was eluted with ethyl acetate. The syrupy 45 was used directly in the next step: 400 MHz ¹H NMR: δ 0.13 (3H, s), 0.14 (3H, s), 0.87 (60H, s), 0.91 (9H, m), 1.10 (1H, m), 1.14 (3H, s), 1.15 (3H, s), 1.21 (61H, s), 1.1-1.6 (19H, m), 1.6-1.9 (5H, m), 1.94 (11H, brd, J=12.8 Hz), 2.05 (3H, s), 3.38 (11H, brs), 5.15 (1H, brs).

### [1R,3aR,4S,7aR]-Acetic acid 1-[4(R)-[dimethyl-(1,1,2-trimethyl-propyl)-silanyloxy]-5-methyl-1(R)-(4-methyl-4-trimethylsilanyloxy-pentyl)-5-trimethylsilanyloxy-hexyl-7a-methyl-octahydro-inden-4-yl ester (46)

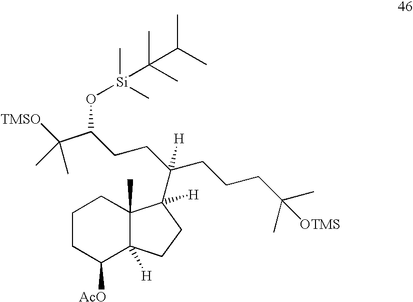

1-(Trimethylsilyl)imidazole (0.90 mL, 6.1 mmol) was added to a solution of 45 (0.2929 mg) in cyclohexane (6 mL) and stirred for 12 h, then flash-chromatographed (1:79 ethyl acetate-hexane) to yield 46 as colorless syrup (0.3372 g). The elution was monitored by TLC (1:4 ethyl acetate-hexane) leading to 46 as a colorless syrup, 0.7915 g: ¹H NMR δ: 0.074 (3H, s), 0.096 (3H, s), 0.103 (9H, s), 0.106 (9H, s), 0.82 (11H, m), 0.83 (6H, s), 0.88 (9H, m), 1.32 (3H, s), 1.20 (9H, s), 1.15-1.6 (17H, m), 1.6-1.9 (5H, m), 1.97 (1H, brd, J=12.8 Hz), 2.05 (3H, s), 3.27 (1H, m), 5.15 (1H, brs); LR-FAB(+) m/z: 712 (M), 711 (M−H), 697 (M-Me), 653 (M-AcO), 627 (M-C₆H₁₃).

### [1R,3aR,4S,7aR]-1-[4R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-ol (47)

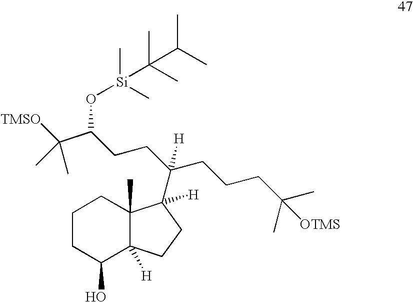

A stirred solution of 46 (0.335 mg, 0.47 mmol) in tetrahydrofuran (15 mL) was cooled in an ice-bath and a 1 M solution of lithium aluminum hydride in tetrahydrofuran (2 mL) was added dropwise. TLC (1:9 ethyl acetate-hexane) showed complete conversion 25b (Rf 0.61) to 26 (Rf 0.29) after 1.5 h. A 2 M sodium hydroxide solution (14 drops) was added, followed by water (0.5 mL) and ethyl acetate (30 mL). A small amount of Celite was added and, after stirring for 15 min, the liquid layer was filtered off. The solid residue was rinsed repeatedly with ethyl acetate and the combined liquid phases evaporated to leave a colorless syrup, that was taken up in hexane, filtered and evaporated to yield 26 (0.335 g) that was used without further purification: ¹H NMR δ: 0.075 (3H, s), 0.10 (211H, brs), 0.82 (11H, m), 0.84 (6H, s), 0.89 (6H, m), 0.93 (3H, s), 1.13 (3H, s), 1.20 (9H, s), 1.2-1.6 (16H, m), 1.6-1.7 (2H, m), 1.82 (31H, m), 1.95 (1H, brd, J=12.4 Hz), 3.27 (1H, m), 4.08 (1H, brs); LR-FAB(+) m/z: 585 (M-C₆H₁₃), 481 (M-TMSO); HR-ES(+) m/z: Calcd for C₃₇H₇₈O₄Si₃+Na: 693.5100 found: 693.5100.

### [1R,3aR,7aR]-1-[4(R)-[Dimethyl-(1,1,2-trimethyl-propyl)-silanyloxy]-5-methyl-1(R)-(4-methyl-4-trimethylsilanyloxy-pentyl)-5-trimethylsilanyloxy-hexyl]-7a-methyl-octahydro-inden-4-one (48)

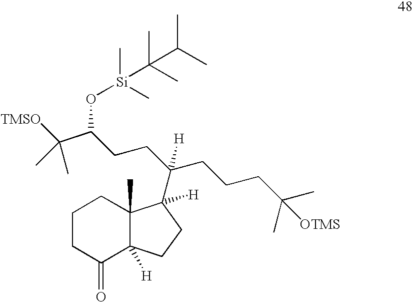

Celite (0.6 g) was added to a stirred solution of 47 (0.310 g, 0.462 mmol) in dichloromethane (14 mL) followed by pyridinium dichromate (0.700 g, 1.86 mmol). The conversion of 47 (Rf 0.54) to the ketone 27 (Rf 0.76) was followed by TLC (1:4 ethyl acetate-hexane). The mixture was diluted with cyclohexane after 4.5 h then filtered through a layer of silica gel. Filtrate and ether washes were combined and evaporated. The residue was flash-chromatographed (1:39 ethyl acetate-hexane) to give 27 as a colorless syrup, 0.2988 g, 96.6%: ¹H NMR δ: 0.078 (3H, s), 0.097 (3H, s), 0.107 (18H, s), 0.64 (3H, s), 0.81 (1H, m), 0.84 (6H, s), 0.89 (6H, m), 1.134 (3H, s), 1.201 (3H, s), 1.207 (3H, s), 1.211 (3H, s), 1.3-1.6 (14H, m), 1.6-1.7 (3H, m), 1.88 (1H, m), 2.04 (2H, m), 2.2-2.32 (2H, m), 2.46 (1H, dd, J=7.5 and 11.5 Hz), 3.28 (1H, m); LR-FAB(+) m/z: 583 (M-C₆H₁₃), 479 (M-OTMS); HR-ES(+) m/z: Calcd for C₃₇H₇₆O₄Si₃+Na: 691.4943, found: 691.4949.

### [1R,3aR,7aR,4E]-4-{2(Z)-[3(S),5(R-Bis-(tert-butyl-dimethyl-silanyloxy)-2-methylene-cyclohexylidene]-ethylidene}-7a-methyl-1-[5-methyl-(]3-(4-methyl-4-trimethylsilanyloxy-pentyl)-4(R)-[dimethyl-(1,1,2-trimethyl-propyl)-silanyloxy]-5-trimethylsilanyloxy-hexyl]-octahydro-indene (49)

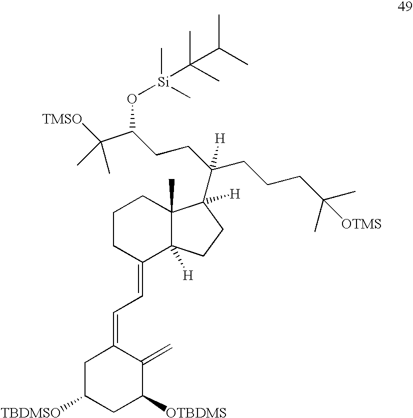

A solution of 2.5-M butyllithium in hexane (0.17 mL) was added to a solution of 28 in tetrahydrofuran (2 mL) at −70° C. to produce a deep cherry-red color of the yield. After 10 min a solution of ketone 27 (0.1415 g, 0.211 mmol) in tetrahydrofuran (2 mL) was added dropwise over a 15 min period. The reaction was quenched after 4 h by the addition of pH 7 phosphate buffer (2 mL). The temperature was allowed to increase to 0° C. then hexane (30 mL) was added. The aqueous layer was re-extracted with hexane (15 mL). The combined extracts were washed with of brine (5 mL), dried and evaporated to give a colorless oil that was purified by flash-chromatography (1:100 ethyl acetate-hexane) to yield 49 as colorless syrup, 0.155 g, 71%: ¹H NMR δ: 0.068 (15H, m), 0.103 (12H, s), 0.107 (9H, s), 0.53 (3H, s), 0.82 (11H, m), 0.84 (6H, s), 0.88 (18H, m), 0.89 (6H, m), 1.14 (3H, m), 1.20 (9H, s), 12-1.9 (22H, m), 1.97 (2H, m), 2.22 (1H, dd, J=7.5 an 13 Hz), 2.45 (1H, brd, J=13 Hz), 2.83 (1H, brd, J=13 Hz), 3.28 (1H, m), 4.20 (1H, m), 4.38 (1H, m), 4.87 (1H, d, J=2 Hz), 5.18 (1H, d, J=2 Hz), 6.02 (1H, d, J=11.4 Hz, 6.24 (1H, d, J=11.4 Hz); LR-FAB(+) m/z 1033 (M+H), 1032 (M), 1031 (M−H), 901 (M-TBDMS).

### Synthesis of 1,25-Dihydroxy-21-(2R,3-dihydroxy-3-methyl-butyl)-20R-Cholecalciferol (33)

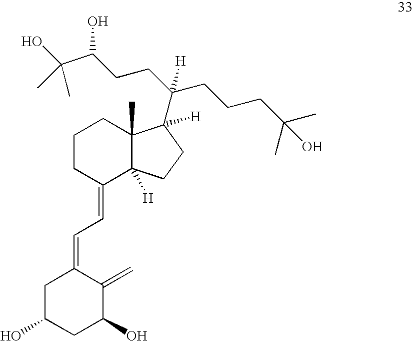

The residue of 49 (0.153 g, 0.148 mmol), as obtained in the previous experiment, was dissolved in a 1 M solution of tetrabutylammonium fluoride (3.5 mL). TLC (ethyl acetate) monitored reaction progress. Thus, the solution was diluted with brine (5 mL) after 24 b, stirred for 5 min then equilibrated with ethyl acetate (35 mL) and water (15 mL). The aqueous layer was re-extracted once with ethyl acetate (15 mL). The combined organic layers were washed with water (5×10 mL), once with brine (5 mL) then dried and evaporated. The residue was purified by flash chromatography using a stepwise gradient of ethyl acetate and 1:100 methanol-ethyl acetate furnishing 33 as colorless, microcrystalline material from methyl formate-pentane, 70 mg, 91%: [α]_{D}+34.3° (methanol, c 0.51); ¹H NMR (DMSO-d₆) δ: 0.051 (3H, s), 0.98 (3H, s), 1.03 (3H, s), 1.05 (6H, s), 1.0-1.6 (17H, m), 1.64 (3H, m), 1.80 (2H, m), 1.90 (1H, d, J=11.7 Hz), 1.97 (1H, dd, J=J=9.8 Hz), 2.16 (1H, dd, J=5.9 and J=13.7 Hz), 2.36 (1H, brd), 2.79 (1H, brd), 3.00 (1H, dd, J=5 and 10 Hz), 3.99 (1H, brs), 4.01 (1H, s, OH), 4.04 (1H, s, OH), 4.54 (11H, OH, d, J=3.9 Hz), 4.76 (1H, brs), 4.87 (11H, OH, d, J=4.9 Hz), 5.22 (1H, brs), 5.99 (1H, d, J=10.7 Hz), 6.19 (1H, d, J=10.7 Hz); LR-ES(+) m/z: 519 (M+H), 518 (M), 517 (M−H), 501 (M-OH); HR-ES(+) calcd for C₃₂H₅₄O₅+Na: 541.3863; found 541.3870; UVₘₐₓ (ε): 213 (13554), 241sh (12801), 265 (16029) nm.

### Example 42

### Synthesis of 1,25-Dihydroxy-21(2R,3-dihydroxy-3-methyl-butyl)-20S-Cholecalciferol (50)

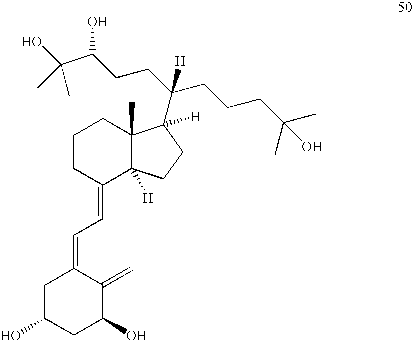

### [1R,3aR,4S,7aR]-7-Benzenesulfonyl-6(R)-[4-(tert-butyl-dimethyl-silanyloxy)-7a-methyl-octahydro-inden-1-yl]-2-methyl-heptan-2-ol (51)

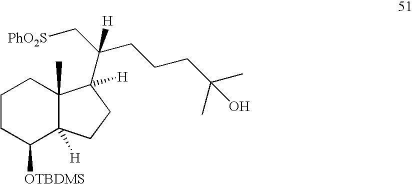

A solution of 36 and sodium benzenesulfinate (0.263 g, 1.6 mmol) in N,N-dimethyl formamide (5 mL) was stirred in a 77° C. bath for 3 h. The solution was equilibrated with 1:1 ethyl acetate-hexane (25 mL) and the organic layer washed with water (5×10 mL), dried and evaporated. The residue was flash-chromatographed with a stepwise gradient of 1:9, 1:4, and 1:3 ethyl acetate-hexane to furnish the sulfone as a colorless syrup: ¹H NMR δ −0.02 (3H, s), 0.005 (3H, s), 0.79 (3H, s), 0.87 (9H, s), 1.12 (1H, m), 1.19 (6H, s), 1.12 (1H, m), 1.20 (6H, s), 1.2-1.8 (18H, m), 2.08 (1H, m), 3.09 (1H, dd, J=9.3 and 14.5 Hz), 3.31 (1H, dd, J=3 and 14.5 Hz), 3.97 (1H, brs), 7.58 (3H, m), 7.66 (1H, m), 7.91 2H, m); LR-ES(+) m/z: 600 (M+Na+MeCN), 559 (M+Na); LR-ES(−) m/z: 536 (M), 535 (M−H); HR-ES(+): Calcd for C₃₀H₅₂O₄SSi+Na 559.3248; found 559.3253.

### [1R,3aR,4S,7aR]-1-(1(R)-Benzenesulfonylmethyl-5-methyl-5-trimethylsilanyloxy-hexyl)-4-(tert-butyl-dimethyl-silanyloxy)-7a-methyl-octahydro-indene (52)

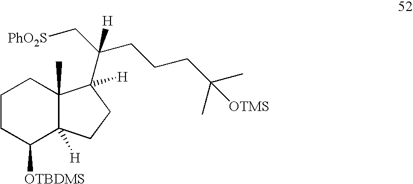

1-(Trimethylsilyl)imidazole (0.146 mL) was added to a solution of 51 (0.145 g, 0.27 mmol) in cyclohexane (2 mL). After 17 h the product was purified by flash chromatography using a stepwise gradient of 1:79 and 1:39 ethyl acetate-hexane to give 52 as colorless residue, 0.157 g 0.258 mmol, TLC (1:9 ethyl acetate-hexane) Rf 0.14. 300 MHz ¹H NMR: δ −0.02 (3H, s), 0.00 (3H, s), 0.87 (12H, s), 1.12 (1H, m), 1.17 (6H, s), 1.2-1.6 (15H, m), 1.6-1.9 (3H, m), 3.08 (2H, m), 3.97 (1H, brs), 7.53-7.70 (3H, m), 7.90 (2H, d, J=7 Hz).

### [1R,3aR4S,7aR]-5(R,S)-Benzenesulfonyl-6(R)-[4-(tert-butyl-dimethyl-silanyloxy)-7a-methyl-octahydro-inden-1-yl]-2,10-dimethyl-10-trimethylsilanyloxy-undecane-2,3(R)-diol (53)

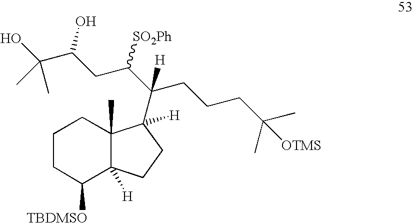

A solution of 152 (0.2589, 0.425 mmol) and diol (0.176 g, 0.638 mmol) in tetrahydrofuran (9 mL) was cooled to −25° C. and 1.6 M butyllithium in hexane (1.4 mL) was added. The temperature was raised to −20° C. and maintained for 3 h then at −10° C. for 2.5 h and 0° C. for 10 min. The mixture was cooled again to −10° C., saturated ammonium chloride solution (5 mL) was added, then equilibrated with ethyl acetate (50 mL) and enough water to dissolve precipitated salts. The aqueous layer was re-extracted with ethyl acetate (15 mL), the combined extracts were dried and evaporated and the residue purified by flash chromatography using a stepwise gradient of 1:6, 1:4, and 1:1 ethyl acetate-hexane to produce 53 as a colorless syrup, 0.212 g, 70%: 300 MHz ¹H NMR: δ 0.00 (3H, s), 0.017 (3H, s), 0.12 (9H, s), 0.81 (3H, s), 0.89 (9H, s), 1.16 (1H, m), 1.19 (12H, m), 1.1-1.6 (20H, m), 1.6-1.8 (2H, m), 3.10 (1H, dd, J=8.4 and 14.7 Hz), 3.30 (1H, m), 3.99 (1H, brs), 7.61 (2H, m), 7.67 (1H, m), 7.93 (2H, m).

### [1R,3aR,4S,7aR]-6(S)-[4-(tert-Butyl-dimethyl-silanyloxy)-7a-methyl-octahydro-inden-1-yl]-2,10-dimethyl-10-trimethylsilanyloxy-undecane-2,3(R)-diol (54)

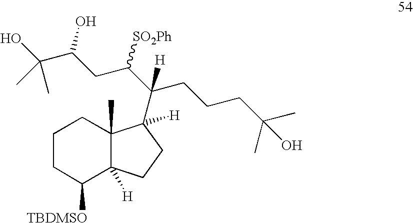

Compound 53 (0.186 mg, 0.262 mmol) was dissolved in 0.5 M oxalic acid dihydrate in methanol (2.5 mL). The solution was stirred for 15 min then calcium carbonate was added (0.5 g) and the suspension stirred overnight then filtered. The filtrate was evaporated to give 54 as a white foam, 0.188 g, 98%: TLC (1:1 ethyl acetate-hexane) Rf 0.06. This material was used in the next step without further purification.

### [1R,3aR,4S,7aR]-6(S)-[4-(tert-Butyl-dimethyl-silanyloxy)-7a-methyl-octahydro-inden-1-yl]-2,10-dimethyl-undecane-2,3(R),10-triol (triol 55)

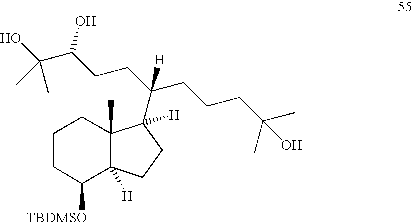

Sodium amalgam (5% sodium, 10.8 g) was added to a vigorously stirred solution of 54 (0.426 g, 0.667 mmol) in a mixture of tetrahydrofuran (15 mL) and methanol (9 mL). The suspension was stirred for 24 h and the reaction monitored by TLC (1:1 ethyl acetate-hexane 0 to observe the production of 55 (Rf 0.17). The mixture was diluted with methanol (3 mL), stirred for 5 min then further diluted with water (10 mL), stirred for 2 min and decanted into saturated ammonium chloride solution (25 mL). The aqueous layer was extracted with ethyl acetate (2×20 mL). The combined extracts were washed with pH 7 phosphate buffer (5 mL) then brine (10 mL), dried and evaporated. The residue was purified by flash-chromatography using a stepwise gradient of 1:1 and 2:1 ethyl acetate-hexane to provide 55 as a colorless syrup, 0.244 g, 73%: ¹H NMR: δ −0.006 (3H, s), 0.006 (3H, s), 0.86 (9H, s), 0.92 (3H, s), 1.11 (1H, m), 1.15 (3H, s), 1.21 (9H, s), 1.2-1.75 (21H, m), 1.7-1.85 (3H, m), 1.90 (1H, m), 3.29 (1H, brd), 3.99 (1H, brs); LR-ES(+) m/z: 521 (M+Na), 481 (M-OH); LR-ES(−): m/z 544: (M+CH₂O₂), 543 (M−H+CH₂O₂), 533 (M−Cl); HR-ES(+) m/z: Calcd for C₂₉H₅₈O₄Si+Na: 521.3996, found 521.3999.

### [1R,3aR,4S,7aR]-6(S)-(4-Hydroxy-7a-methyl-octahydro-inden-1-yl)-2,10-dimethyl-undecane-2,3(R),10-triol (56)

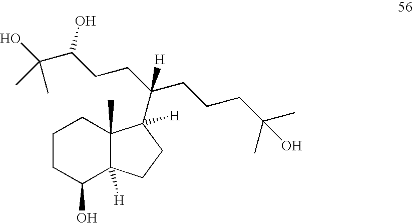

An aqueous fluorosilicic acid solution (3 mL) was added to a stirred solution of 55 (0.240 g, 0.481 mmol) in acetonitrile (1.2 mL). TLC (ethyl acetate) monitored the reaction. After 2.5 h. compound 56 (Rf 0.37) was the predominating species, produced at the expense of less polar 55. The mixture was equilibrated with ethyl acetate and water (10 mL), the aqueous layer was re-extracted with water (2×10 mL) and the combined extracts were washed with water (6 mL) and brine (2×10 mL) then dried and evaporated. The colorless residue was flash-chromatographed using a stepwise gradient of 1:2, 1:1 and 2:1 ethyl acetate-hexane to elute some unreacted 55, followed by 56, obtained as colorless syrup, 0.147 g, 79%: ¹H NMR: 0.94 (3H, s), 1.12 (1H, m), 1.15 (3H, s), 1.21 (9H, s), 1.15-1.7(20H, m), 1.7-1.9(5H, m), 1.96(1H, brd), 3.29(1H, d, J=9.6 Hz), 4.08 (1H, brs); LR-ES(+): m/z 448: (M+Na+MeCN), 407 (M+Na); LR-ES(−): m/z 419 (M+Cl); HR-ES(+) i/z: Calcd for C₂₃H₄₄O₄+Na: 407.3132, found 407.3135.

### [1R,3aR,4S,7aR]-1-(5-Hydroxy-1(5)-{2-[2-(4-methoxy-phenyl)-5,5-dimethyl-[1,3]dioxolan-4(R)-yl]-ethyl}-5-methyl-hexyl)-7a-methyl-octahydro-inden-4-ol (57)

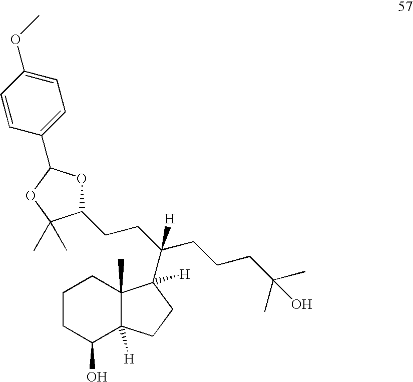

4-Methoxybenzaldehyde dimethyl acetal (60 uL, 0.35 mmol) was added to a solution of 56 (81.2 mg, 0.211 mmol) in dichloromethane (2 mL), followed by a solution (0.2 mL) containing pyridinium tosylate (200 mg) in dichloromethane (10 mL). Reaction progress was followed by TLC (1:2 ethyl acetate-hexane) which showed 4-methoxybenzaldehyde dimethyl acetal (Rf 0.80), 4-methoxybenzaldehyde (Rf 0.65), educt 56 (Rf 0.42) and product 57 (Rf 0.26). After 5¾ h the mixture was stirred for 15 min with saturated sodium hydrogencarbonate solution (5 mL) then equilibrated with ethyl acetate (25 mL). The organic layer was washed with brine (5 mL), dried and evaporated. The residue was flash-chromatographed using a stepwise gradient of 1:3 and 1:2 ethyl acetate-hexane to yield 57 as colorless syrup, 0.106 mg (100%): ¹H NMR: 0.94 (3H, s), 1.19, 1.21 (6H, s each, Me₂COH), 1.23, 1.35 and 1.24, 1.37 (6H, s each, major and minor 5,5-dimethyloxolane diastereomer), 1.1-1.7 (188H, m), 1.7-1.9 (5H, m), 1.9-2.0 (2H, m), 3.65 (1H, m), 3.81 (3H, s), 4.08 (1H, brs), 5.78 and 5.96 (1H, s each, major and minor acetal diastereomer), 6.89 (2H, m), 7.41 (2H, m).

### [1R,3aR,7aR]-1-(5-Hydroxy-[(S)-{2-[2-(4-methoxy-phenyl)-5,5-dimethyl-[1,3]dioxolan-4(R)-yl]-ethyl}-5-methyl-hexyl)-7a-methyl-octahydro-inden-4-one (58)

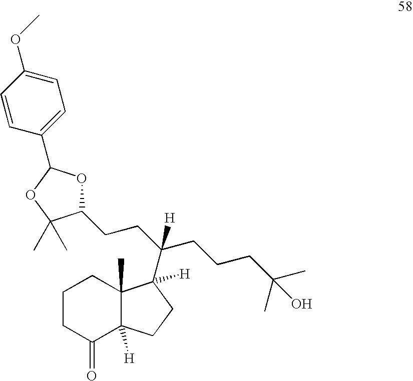

Pyridinium dichromate (230 mg, 0.61 mmol) was added to a stiffed mixture containing 57 (0.0838, 0.167 mmol), Celite (185 mg), and dichloromethane (4 mL). The conversion of 57 (Rf 0.31) to 58 (Rf 0.42) was monitored by TLC (1:25 methanol-chloroform) The mixture was diluted with dichloromethane (10 mL) after 2.5 h, then filtered through a layer of silica gel. Filtrate and washings (1:1 dichloromethane-ethyl acetate) were evaporated and the residue chromatographed (1:4 ethyl acetate-hexane) to give ketone 58, 0.0763 g, 91%: ¹H NMR: 0.63 (3H, s), 1.19, 1.21 and 1.23 (6H, s each, Me₂COH), 1.25, 1.36, 1.38 (6H, m, s, s, 5,5-dimethyloxolane diastereomer), 1.1-1.9 (18H, m), 1.9-2.1 (3H, m), 2.1-2.4 (2H, m), 2.45 (1H, m), 3.66 (1H, m), 3.802 and 3.805 (3H, s each), 5.78 and 5.95 (1H, s each, major and minor acetal diastereomer), 6.89 (2H, m), 7.39 (2H, m).

### [1R,3aR,7aR]-1-[4(R),5-Dihydroxy-1(S)-(4-hydroxy-methyl-pentyl)-5-methyl-hexyl]-7a-methyl-octahydro-inden-4-one (59)

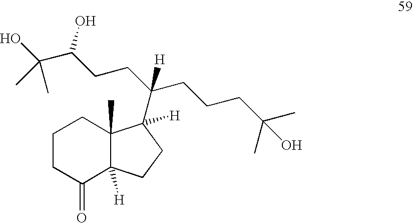

The ketone 58 was stirred in a 1 N oxalic acid solution in 90% methanol. The mixture became homogeneous after a few min. TLC (ethyl acetate) suggested complete reaction after 75 min. (f 0.24 for 59). Thus, calcium carbonate (0.60 g) was added and the suspension stirred overnight, then filtered. The filtrate was evaporated and flash-chromatographed using a stepwise gradient of 4:1:5 dichloromethane-ethyl acetate-hexane, 1:1 ethyl acetate-hexane, and neat ethyl acetate produce 59 as a colorless residue, 0.060 mg, 94%: ¹H NMR: 0.5 (3H, s), 1.17 (3H, s), 1.22 (6H, s), 1.23 (3H, s), 1.2-1.21 (23H, m), 2.15-2.35 (2H, m), 2.45 (1H, dd, J=7 and 11 Hz), 3.30, 1H, brd).

### [1R,3aR,7aR]-7a-Methyl-1-[5-methyl-1S-4-methyl-4-triethylsilanyloxy-pentyl)-4(R),5-bis-triethylsilanyloxy-hexyl]-octahydro-inden-4-one (60)

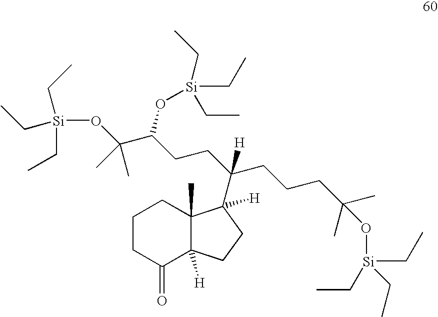

A mixture of 59 (0.055 g, 0.143 mmol), imidazole, (14.9 mg, 1.69 mmol), N,N-dimethylpyridine (6 mg), triethylchlorosilane (0.168 mL, 1 mmol) and N,N-dimethylformamide (1.5 mL) was stirred for 17 h. The reaction was followed by TLC (1:4 ethyl acetate-hexane) and showed rapid conversion to the disilyl intermediate (Rf 0.47). Further reaction proceeded smoothly overnight to give the fully silylated 60 (Rf 0.90). The solution was equilibrated with water (3 mL), equilibrated with ethyl acetate (20 mL), the ethyl acetate layer was washed with water (3×4 mL), dried and evaporated. The residue was flash-chromatographed using a stepwise gradient of hexane and 1:100 ethyl acetate-hexane to yield 60 as a colorless syrup, 0.0813 g, 78.4%: ¹H NMR δ 0.55-0.64 (2H, m), 0.92-0.97 (27H, m), 1.12 (3H, s), 1.18 (3H, s), 1.19 (3H, s), 1.21 (3H, s), 1.1-1.7 (18H, m), 1.9-2.15 (2H, m), 2.15-2.35 (2H, m), 2.43 (1H, dd, J=7.7 and 11 z), 3.30 (1H, dd, J=3 and 8.4 Hz).

### [1R,3aR,7aR,4E]-4-{2(Z)-[3(S),5(R)-Bis-(tert-butyl-dimethyl-silanyloxy)-2-methylene-cyclohexylidene]-ethylidene}-7a-methyl-1-[5-methyl-1(S)-(4-methyl-4-triethylsilanylox-pentyl)-4(R),5-bis-triethylsilanyloxy-hexyl]-octahydro-indene (61)

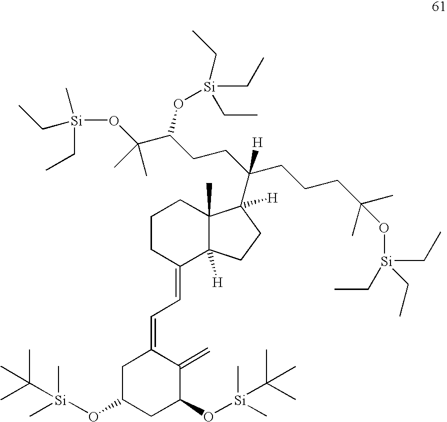

A solution of 1.6 M butyllithium in hexane (0.14 mL) was added to a solution of phosphine (0.1308 g, 0.224 mmol) in tetrahydrofuran (1.5 mL) at −70° C. After 10 min a solution of ketone 60 (0.0813 g, 0.112 mmol) in tetrahydrofuran (1.5 mL) was added dropwise over a 15 min period. The ylide color had faded after 3 h so that pH 7 phosphate buffer (2 mL) was added and the temperature allowed to increase to 0° C. The mixture was equilibrated with hexane (30 mL), the organic layer was washed with brine (5 mL), dried and evaporated to give a colorless oil that was purified by flash-chromatography (1:100 ethyl acetate-hexane). Only the band with Rf 0.33 (TLC 1:39 ethyl acetate-hexane) was collected. Evaporation of those fractions gave 61 as colorless syrup, 0.070 g, 57%: ¹H NMR δ 0.06 (12H, brs), 0.53-0.64 (21H, m), 0.88 (18H, s), 0.92-0.97 (27H, m), 1.11 (3H, s), 1.177 (3H, s), 1.184 (3H, s), 1.195 (3H, s), 1-1.9 (22H, m), 1.98 (2H, m), 2.22 (1H, m), 2.45 (1H, m), 2.83 (1H, brd, J=13 Hz, 3.27 (1H, d, J=6 Hz), 4.19 (1H, m), 4.38 (1H, m), 4.87 (1H, brs), 5.18 (1H, brs), 6.02 (1H, d, J=11 Hz), 6.24 (1H, d, J=11 Hz).

### Synthesis of 1,25-Dihydroxy-21(2R,3-dihydroxy-3-methyl-butyl)-20S-Cholecalciferol (50)

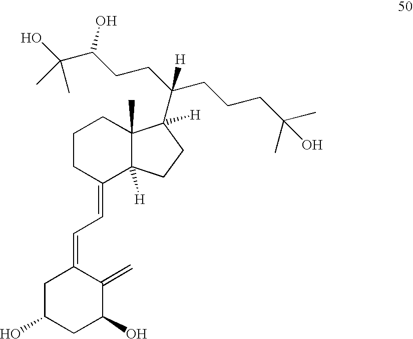

The deprotection reaction of 61 (0.068 g, 0.06238 mmol) in 1M solution of tetrabutylammonium fluoride in tetrahydrofuran, followed by TLC (ethyl acetate), gradually proceeded to give 50 (Rf 0.19). The mixture was diluted with brine (5 mL) after 25 h, stirred for 5 min the equilibrated with ethyl acetate (35 mL) and water (15 mL). The aqueous layer was re-extracted once with ethyl acetate (35 mL), the combined extracts were washed with water (5×10 mL) and brine (5 mL) then dried and evaporated. The residue was flash-chromatographed using a linear gradient of 1:1 and 2:1 ethyl acetate-hexane, and 2:98 methanol-ethyl acetate to give a residue that was taken up in methyl formate and evaporated to a white foam, 30 mg, 93%: [α]_{D} +29.3° (methanol, c 0.34); MHz ¹H NMR δ: 0.55 (3H, s), 1.16 (3H, s), 1.21 (9H, s), 1.1-1.75 (22H, m), 1.80 (2H, m), 1.9-2.1 (5H, m), 2.31 (1H, dd, J=7 and 13 Hz), 2.60 (1H, brd), 284 (1H, m), 3.29 (1H, d, J=9.5 Hz), 4.22 (1H, m), 4.43 (1H, m), 5.00 (1H, s), 5.33 (1H, s), 6.02 (1H, d, J=11 Hz), 6.02 (1H, d, J=11Hz); LR-ES(−) m/z: 564 (M+H₂CO₂), 563 M−-H+H2CO2); HR-ES(+) calcd for C₃₂H₅₄O₅+Na: 541.3863; found 541.3854; UVₘₐₓ (ε): 211 (15017), 265 (15850), 204 sh (14127), 245 sh (13747) nm.

### Example 43

### Synthesis of 1,25-Dihydroxy-21-(2R,3-dihydroxy-3-methyl-butyl)-20S-19-nor-cholecalciferol (62)

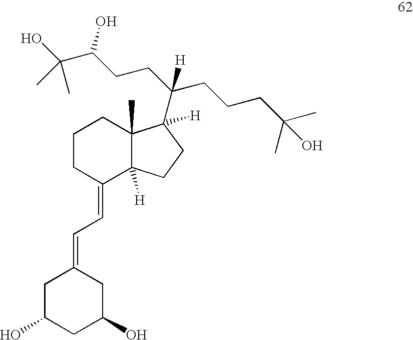

### [1R,3aR,7aR,4E]-4-{2(Z)-[3(S),5(R)-Bis-(tert-butyl-dimethyl-silanyloxy)-cycloheylidene]-ethylidene}-7a-methyl-1-[5-methyl-1(S)-(4-methyl-4-triethylsilanyloxy-pentyl)-4(R),5-bis-triethylsilanyloxy-hexyl]-octahydro-indene (63)

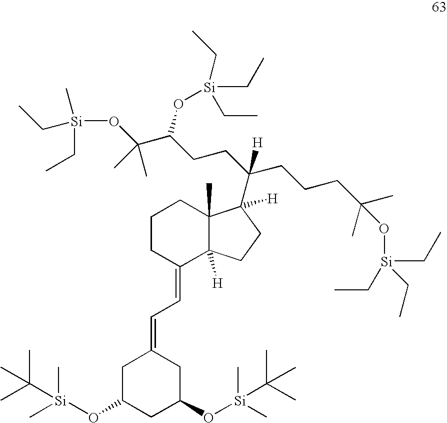

A solution of 1.6 M butyllithium in hexane was added to a solution of phosphine in tetrahydrofuran at −70° C. After 10 min a solution of ketone 60 from Example 2 in tetrahydrofuran was added dropwise over a 15 min period. After the ylide color had faded, pH 7 phosphate buffer was added and the temperature allowed to increase to 0° C. The mixture was equilibrated with hexane, the organic layer was washed with brine, dried and evaporated to give a colorless oil that was purified by flash-chromatography (1:100 ethyl acetate-hexane) that gave 63.

### 1,25-Dihydroxy-21-(2R,3-dihydroxy-3-methyl-butyl)-20S-19-nor-cholecalciferol (62)

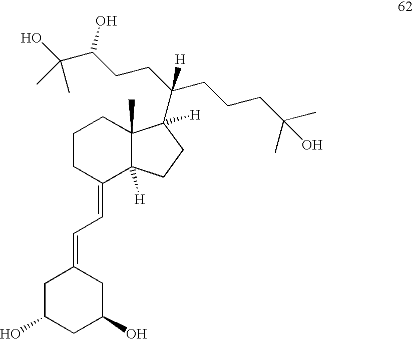

The deprotection reaction of 63 was carried out in 1M solution of tetrabutylammonium fluoride in tetrahydrofuran to give 62. The mixture was diluted with brine after 25 h, stirred for 5 min and then equilibrated with ethyl acetate and water. The aqueous layer was re-extracted once with ethyl acetate, the combined extracts were washed with water and brine, and then dried and evaporated. The residue was flash-chromatographed to give a residue that was taken up in methyl formate and evaporated to yield 62.

### Example 44

### Synthesis of 1,25-dihydroxy-20S-21(3-hydroxy-3-methyl-butyl)-24-keto-19-nor-cholecalciferol (64)

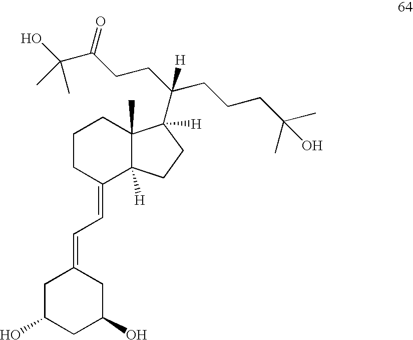

### (R-6-[1(1R,3aR,4S,7aR)-4-(tert-Butyl-dimethyl-silanyloxy)-7a-methyl-octahydro-inden-1-yl]-2-methyl-7-phenylsulfanyl-heptan-2-ol (65)

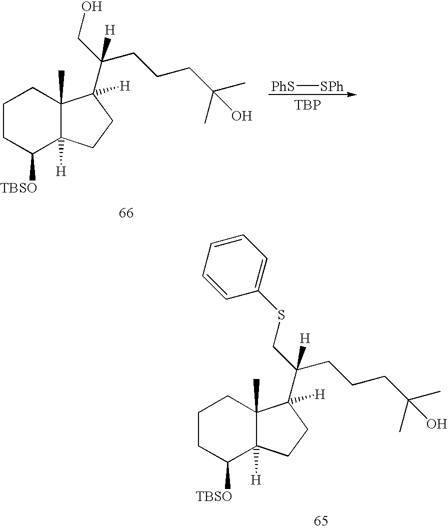

The reaction above was carried out as described in Tet. Lett. 1975, 17: 1409-12. Specifically, a 50 mL round-bottom flask was charged with 1.54 g (3.73 mmol) of (R)-2-[(1R,3aR,4S,7aR)-4-(tert-Butyldimethylsilanyloxy)-7a-methyloctahydroinden-1-yl]-6-methylheptane-1,6-diol (1) (Eur. J. Org. Chem. 2004, 1703-1713) and 2.45 g (11.2 mmol) of diphenylsulfide. The mixture was dissolved in 5 mL of pyridine and 2.27 g (11.2 mmol, 2.80 mL) of tributylphosphine was added. The mixture was stirred overnight and then diluted with 20 mL of toluene and evaporated. The residue was again taken up in toluene and evaporated, the remaining liquid chromatographed on silica gel using stepwise gradients of hexane, 1:39, 1:19 and 1:9 ethyl acetate-hexane to provide the title compound 65 as a syrup, 1.95 g.

### (R)-7-Benzenesulfonyl-6-[(1R,3aR,4S,7aR)-4-(tert-butyl-dimethyl-silanyloxy)-7a-methyl-octahydro-inden-1-yl]-2-methyl-heptan-2-ol (67) and (1R,3aR,4S,7aR)-1-(R)-1-Benzenesulfonylmethyl-5-methyl-5-triethylsilanyloxy-hexyl)-4-(tert-butyl-dimethyl-silanyloxy)-7a-methyl-octahydro-indene (68)

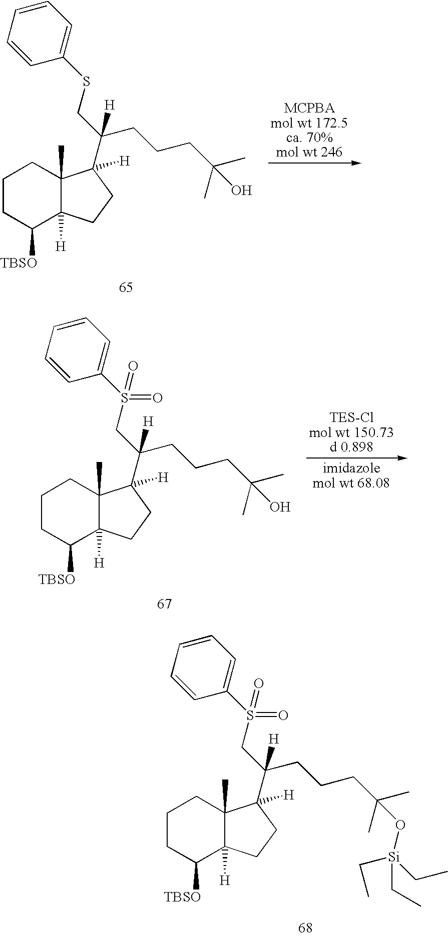

A 500-mL round-bottom flask containing 1.95 g (3.9 mmol) of the crude sulfide 65 was admixed with 84 g of dichloromethane (63 mL). The solution was stirred in an ice bath, then 2.77 g (11 mmol) of meta-chloroperbenzoic acid was added in one portion. The suspension was stirred in the ice bath for 40 min then at room temperature for 2 h. The reaction was monitored by TLC (1:19 methanol-dichloromethane). At the end of the reaction period, only one spot at Rf 0.45 observed. Then, 1.68 g (20 mmol) of solid sodium hydrogen carbonate was added to the suspension, the suspension was stiffed for 10 min, then 30 mL of water was added in portions and vigorous stirring continued for 5 min to dissolve all solids. The mixture was further diluted with 40 mL of hexane, stirred for 30 min, transferred to a separatory funnel with 41.6 g of hexane. The lower layer was discarded and the upper one was washed with 25 mL of saturated sodium hydrogen carbonate solution, dried (sodium sulfate) and evaporated to give 3.48 g of 67. This material was triturated with hexane, filtered, and evaporated, to leave 67 as a cloudy syrup (2.81 g) that was used directly in the next step.

A 100-mL round bottom flask containing 2.81 g of 67 obtained above, was charged with 30 mL of N,N-dimethylformamide 1.43 g of (21 mmol) of imidazole and 1.75 mL of (10 mmol) of triethylsilyl chloride. The mixture was stirred for 17 h then diluted with 50 g of ice-water, stirred for 10 min, further diluted with 5 mL of brine and 60 mL of hexane. The aqueous layer was re-extracted with 20 mL of hexane, both extracts were combined, washed with 2×30 mL of water, dried, evaporated. This material contained a major spot with Rf 0.12 (1:39 ethyl acetate-hexane) and a minor spot with Rf 0.06. This material was chromatographed on silica gel using hexane, 1:100, 1:79, 1:39 and 1:19 ethyl acetate-hexane as stepwise gradients. The major band was eluted with 1:39 and 1:19 ethyl acetate-hexane to yield 1.83 g of 68.

### (R)-5-Benzenesulfonyl-6-[(1R,3aR,4S,7aR)-4-(tert-butyl-dimethyl-silanyloxy)-7a-methyl-octahydro-inden-1-yl]-10-methyl-2-(R)-methyl-10-triethylsilanyloxy-undecane-2,3-diol (69)

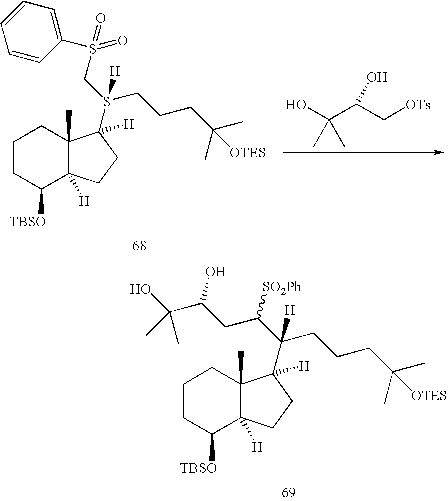

A 100-mL 3-neck round-bottom flask, equipped with magnetic stirrer, thermometer and Claisen adapter with rubber septum and nitrogen sweep, was charged with 1.7636 g of (2.708 mmol) of sulfone 68, 1.114 g of (4.062 mmol) tosylate, and 50 mL of tetrahydrofuran freshly distilled from benzophenone ketyl. This solution was cooled to −20° C. and 9.31 mL of a 1.6 M butyllithium solution in hexane was added dropwise at ≦−20° C. The temperature range between −10 and −20° C. was maintained for 5 h. The cooling bath was removed and 50 mL of saturated ammonium chloride solution added followed by 75 mL of ethyl acetate and enough water to dissolve all salts. The organic layer was washed with 15 mL of brine, dried, and evaporated to a colorless oil. This residue was chromatographed on silica gel using hexane, 1:9, 1:6, 1:4 and 1:3 ethyl acetate-hexane as stepwise gradients. The main band was eluted with 1:4 and 1:3 ethyl acetate-hexane to furnish 1.6872 g of compound 69 as colorless syrup.

### (S)-6-[(1R,3aR,4S,7aR)-4-(tert-Butyl-dimethyl-silanyloxy-7a-methyl-octahydro-inden-1-yl]-10-methyl-2-(R)-methyl-10-triethylsilanyloxy-undecane-2,3-diol (70)

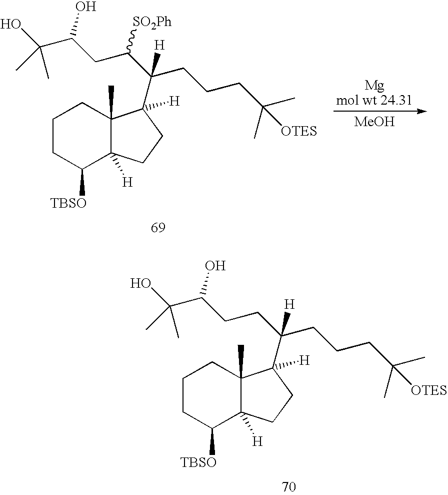

A 25-mL 2-neck round-bottom flask, equipped with magnetic stirrer, thermometer and Claisen adapter with rubber septum and nitrogen sweep, was charged with 1.6872 g (2.238 mmol) of sulfone 69 and 40 mL of methanol. Then 1.25 g (51.4 mmol) of magnesium was added to the stirred solution in two equal portions, in a 30 min time interval. The suspension was stirred for 70 min then another 0.17 g of magnesium and ca. 5 mL of methanol was added and stirring continued 1 h. The mixture was then diluted with 100 mL of hexane and 50 mL of 1 M sulfuric acid was added dropwise to give two liquid phases. The aqueous layer was neutral. The aqueous layer was re-extracted once with 25 mL of 1:1 dichloromethane-hexane. The organic layers were combined then washed once with 15 mL of brine, dried and evaporated. The resulting material was chromatographed on silica gel using hexane, 1:39, 1:19 and 1:9 ethyl acetate-hexane as stepwise gradients. The main band was eluted with 1:9 ethyl acetate-hexane to provide 1.2611 g of 70 as a colorless syrup.

### (S)-6-[(1R,3-R,4S,7aR)-4-(tert-Butyl-dimethyl-silanyloxy)-7a-methyl-octahydro-inden-1-yl]-2,10-dihydroxy-2,10-dimethyl-undecan-3-one (71)

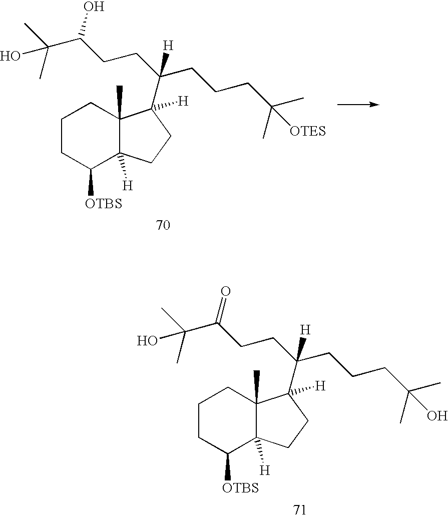

A 25-mL round-bottom flask, equipped with magnetic stirrer, thermometer, Claisen adapter with nitrogen sweep and rubber septum, was charged with 518 mg (3.88 mmol) of N-chlorosuccinamide and 11 mL of toluene. Stir for 5 min (not all dissolved), then cool to 0° C. and add 2.4 mL (4.8 mmol) of a 2M dimethyl sulfide solution in toluene. The mixture was stirred from 5 min then cooled to −30° C. and a solution of 0.7143 g (1.165 mmol) of the diol 70 in 4×1.5 mL of toluene was added dropwise at −30° C. Stirring was continued at this temperature for 1 h. The mixture was then allowed to warm to −10° C. during a 2 h time period then cooled to −17° C. and 3.20 mL (6.4 mmol) of 2 M triethylamine in toluene added dropwise. The mixture was stirred at −17 to −20° C. for 10 ruin then allowed to warm to room temperature slowly. The mixture was chromatographed on a silica gel column using hexane, 1:79, 1:39, 1:19, 1:9, 1:4, and 1:1 ethyl acetate-hexane as stepwise gradients. The major band was eluted with 1:1 ethyl acetate-hexane providing 0.3428 g of the compound 71 as solids.

### (S)-2,10-Dihydroxy-6-((1R,3aR,4S,7aR)-4-hydroxy-7a-methyl-octahydro-inden-1-yl)-2,10-dimethyl-undecan-3-one (72)

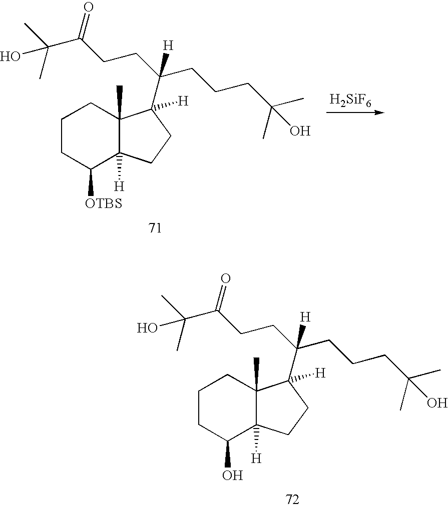

A 25-mL round-bottom flask, equipped with magnetic stirrer was charged with 0.3428 g (0.69 mmol) of the diol 71, was dissolved in 5 mL of acetonitrile then 1.25 mL of fluorosilicic acid solution. After 3 h, the mixture was distributed between 35 mL of ethyl acetate and 10 mL of water, the aqueous layer was re-extracted with 10 mL of ethyl acetate, the organic layers combined, washed with 2×5 mL of water, once with 5 mL of 1:1 brine-saturated sodium hydrogen carbonate solution, dried and evaporated. This material was chromatographed on silica gel using 1:4, 1:3, 1:2, and 1:1 as stepwise gradients furnishing 0.2085 g of the title compound 72.

### (1R,3aR,7aR)-1-[(S)-5-Hydroxy-1-(4-hydroxy-4-methyl-pentyl)-5-methyl-4-oxo-hexyl]-7a-methyl-octahydro-inden-4-one (73)

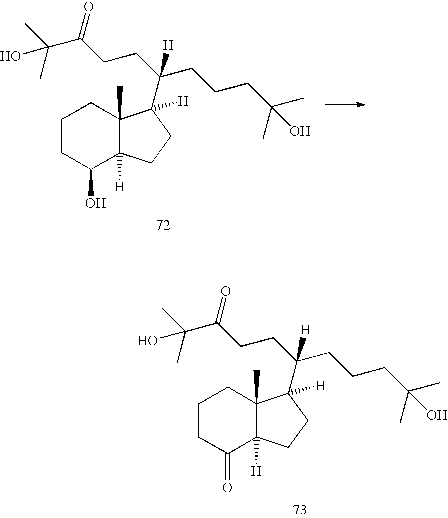

A 25-mL round bottom flask was charged with 0.2153 g (0.56 mmol) of 72, 5 mL of dichloromethane, and 0.20 g of Celite. To this stirred suspension was added, in on portion, 1.00 g (2.66 mmol) of pyridinium dichromate. The reaction stirred for 3 h and the progress was monitored by TLC (1:1 ethyl acetate-hexane). The reaction mixture was diluted with 5 mL of cyclohexane then filtered trough silica gel G. The column was eluted with dichloromethane followed by 1:1 ethyl acetate-hexane until no solute was detectable in the effluent. The effluent was evaporated and the colorless oil. This oil was then chromatographed on a silica gel using 1:4, 1:3, 1:2, 1:1 and 2:1 ethyl acetate-hexane as stepwise gradients to furnish 0.2077 g of the diketone 73.

### (1R,3aR,7aR)-7a-Methyl-1-[(S)-5-methyl-1-(4-methyl-4-trimethylsilanyloxy-pentyl)-4-oxo-5-trimethylsilanyloxy-hexyl]-octahydro-inden-4-one (74)

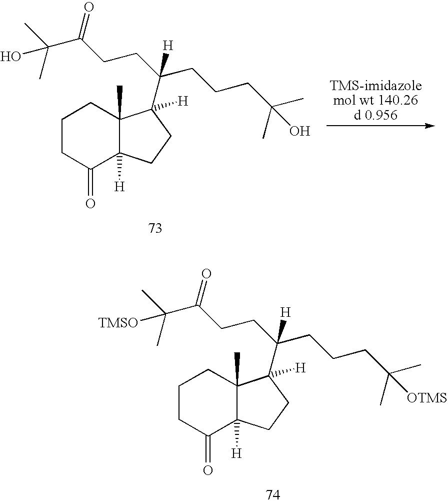

A 25-mL round bottom flask was charged with 0.2077 g (0.545 mmol) of the diketone 73. This material was dissolved in a mixture of 0.5 mL of tetrahydrofuran and 3 mL of cyclohexane. To the resulting mixture was added 0.30 mL (2.0 mmol) of TMS-imidazole. The reaction mixture was diluted with 3 mL of hexane after 10 h then concentrated and chromatographed on silica gel using hexane, 1:79, 1:39, 1:19 and ethyl acetate-hexane as stepwise gradients to provide 0.2381 g of 74 as a colorless oil.

### (S)-6-((1R,3aS,7aR)-4-{2-[(R)-3-((R)-tert-Butyldimethylsilanyloxy)-5-(tert-butyldimethylsilanyloxy)-cyclohexylidene]-ethylidene}-7a-methyloctahydroinden-1-yl)-2,10-dimethyl-2,10-bis-trimethylsilanyloxyundecan-3-one (75)

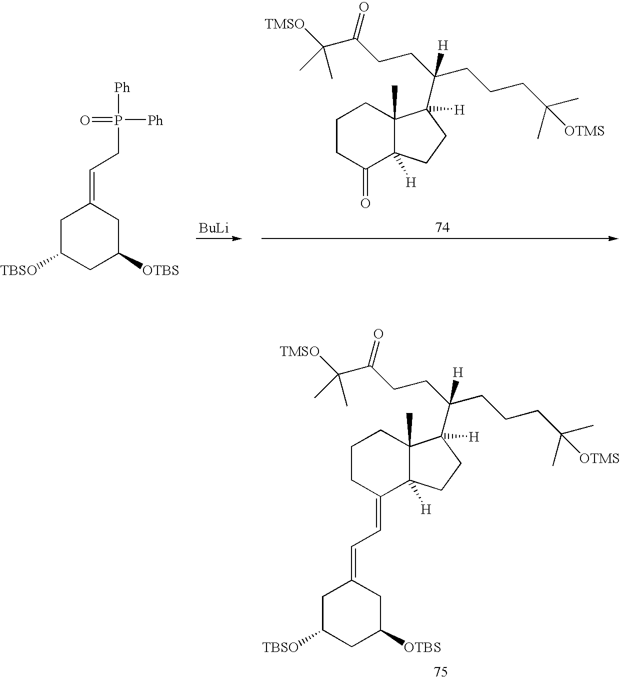

A 15-mL 3-neck pear-shaped flask, equipped with magnetic stirrer, thermometer and a Claisen adapter containing a nitrogen sweep and rubber septum, was charged with 0.2722 g (0.4768 mmol) of [2-[(3R,5R)-3,5-bis(tert-butyldimethylsilanyloxy)cyclohexylidene]ethyl]diphenylphosphine oxide and 2 mL of tetrahydrofuran. The solution was cooled to −70° C. and 0.30 mL of 1.6 M butyllithium in hexane was added. The deep red solution was stirred at that temperature for 10 min then 0.1261 g (0.240 mmol) of the diketone 74, dissolved in 2 mL of tetrahydrofuran was added, via syringe, dropwise over a 10 min period. After 3 h and 15 min, 5 mL of saturated ammonium chloride solution was added at −65° C., the mixture allowed to warm to 10° C. then distributed between 35 mL of hexane and 10 mL of water. The aqueous layer was re-extracted once with 10 mL of hexane, the combined layers washed with 5 ml of brine containing 2 mL of pH 7 buffer, then dried and evaporated. This material was chromatographed on a flash column, 15×150 mm using hexane and 1:100 ethyl acetate-hexane as stepwise gradients to yield 0.1572 g of the title compound 75 as a colorless syrup.

### 1,25-Dihydroxy-20S-21(3-hydroxy-3-methyl-butyl)-24-keto-19-nor-cholecalciferol (64)

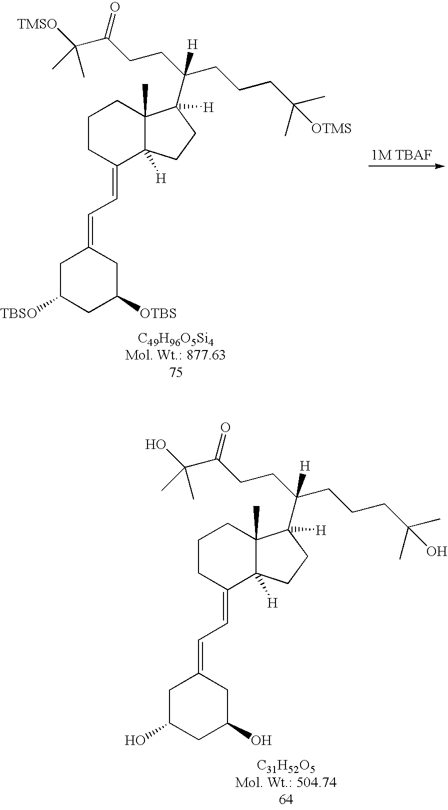

A 15-mL 3-neck round-bottom flask, equipped with magnetic stirrer, was charged with 155 mg (0.17 mmol) of tetrasilyl ether 75. This colorless residue was dissolved is 2 mL of a 1 M solution of tetrabutylammonium fluoride in tetrahydrofuran. After 43 h an additional 0.5 mL of 1 M solution of tetrabutylammonium fluoride solution was added and stirring continued for 5 h. The light-tan solution was the diluted with 5 mL of brine, stirred for 5 min and transferred to a separatory funnel with 50 mL of ethyl acetate and 5 mL of water then re-extraction with 5 mL of ethyl acetate. The organic layers were combined, washed with 5×10 mL of water, 10 mL of brine, dried and evaporated. The resulting residue was chromatographed on a 15×123 mm column using 2:3, 1:1, 2:1 ethyl acetate-hexane, and ethyl acetate as stepwise gradients to provide the 64 as a white solid (TLC, ethyl acetate, Rf 0.23) that was taken up in methyl formate, filtered and evaporated furnishing 0.0753 g of the title compound 64 as a solid substance.

### Example 45

### Synthesis of 1,25-dihydroxy-20S-21(3-hydroxy-3-methyl-butyl)-24-keto-cholecalciferol (76)

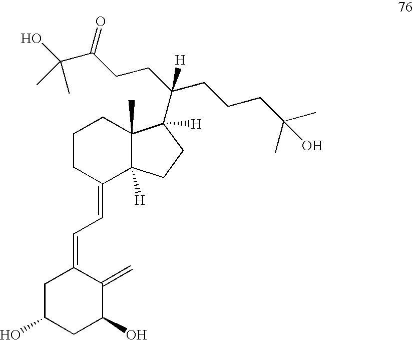

### (S)-6-{(1R,3aS,7aR)-4-[2-[(R)-3-(tert-Butyl-dimethyl-silanyloxy)-5-((S)-tert-butyl-dimethyl-silanyloxy)-2-methylene-cyclohexylidene]-eth-(E)-ylidene]-7a-methyl-octahydro-inden-1-yl}-2,10-dimethyl-2,10-bis-trimethylsilanyloxy-undecan-3-one (77)

Compound 77 was prepared as described for 75 in Example 4 but by reacting 74 with [(2Z)-2-[(3S,5R)-3,5-bis(tert-butyldimethylsilanyloxy)meth-ylenecyclohexylidene]-ethyl]diphenylphosphine oxide.

### 1,25-(Dihydroxy-20S-21(3-hydroxy)-3-methyl-butyl)-24-keto-cholecalciferol (76)

Compound 76 was prepared from 77 by deprotecting 77 as described in Example 22 for 64.

### Example 46

### Synthesis of 1,3-O-Diacetyl-1,25-Dihydroxy-16-ene-24-Keto-19-nor-Cholecalciferol (78)

Referring to Scheme 1 below, compounds of formula I of the invention are prepared as shown in Scheme 1 below. Accordingly, compounds of formula I (wherein X₁ and X₂ are each independently H₂ or ═CH₂, provided X₁ and X₂ are not both ═CH₂; R₁ and R₂ are each independently, hydroxyl, OC(O)C₁-C₄ alkyl, OC(O)hydroxyalkyl or OC(O)fluoroalkyl, provided that R₁ and R₂ are not both hydroxyl; R₃ and R₄ are each independently hydrogen, C₁-C₄ alkyl, or R₃ and R₄ taken together with C₂₀ form C₃-C₆ cycloalkyl; R₅ and R₆ are each independently C₁-C₄ alkyl, hydroxyalkyl, or haloalkyl, e.g., fluoroalkyl, e.g., fluoromethyl and trifluoromethyl) are prepared by coupling compounds of formula II with compounds of formula III in tetrahydrofuran with n-butyllithium as a base to give compounds of formula IV. Subsequent removal of the protecting silyl groups (R₁=OSi(CH₃)₂t.Bu) affords the 1,3 dihydroxy vitamin D₃ compound of formula I (R₁=OH, R₂=OH). Acylation at the 1 and/or 3 positions is achieved using methods well-known in the air. For example, preparation of the 1,3 diacetoxy compounds of formula I (R₁=R₂=OAc) requires additional acetylation with acetic anhydride and pyridine, as shown in Scheme 2.

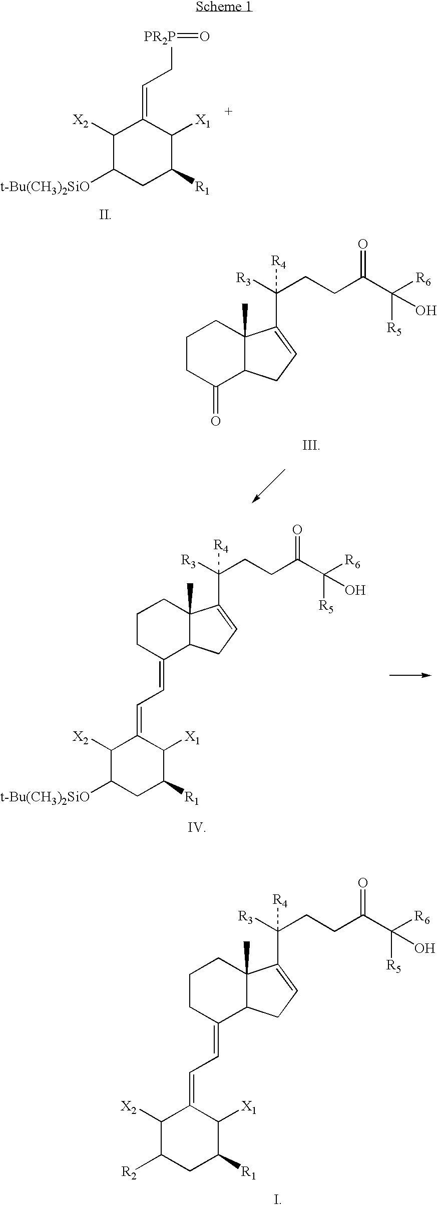

wherein X₁, X₂, R₃, R₄, R₅ and R₆ are as defined above.

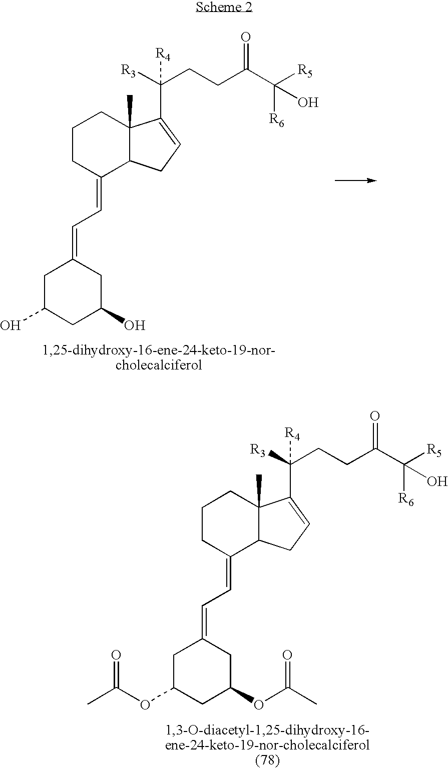

Referring to Schemes 1 and 3, compounds of formula II are known compounds, and are prepared starting from the known epoxy-ketone of formula V. The compound of formula V is converted to the epoxy-olefin of formula VII by a Wittig reaction. Reduction with LiAlH₄ to the compound VIII and protection of the hydroxy group resulted in compound IX. Then, the ene reaction of formula IX with the known hydroxy-conjugated ketone X (R₅=R₆=CH₃) in tetrahydrofuran, in the presence of Lewis acid (CH₃)₂ Al Cl, provides the compound XI featuring the C, D-rings and full side chain of the target vitamin D analogues. Finally, removal of the silyl group and oxidation provides the key intermediate, Ketone of formula III.

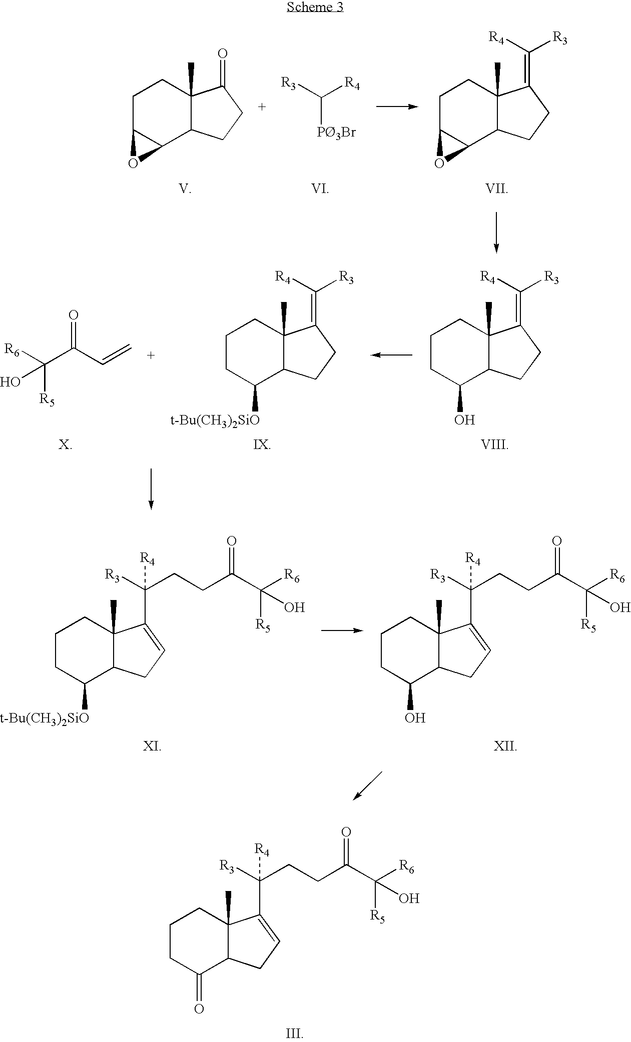

Referring to Scheme 2, 0.032 g of 1,25-dihydroxy-16-ene-24-keto-19-nor-cholecalciferol was dissolved in 0.8 ml pyridine, cooled in bath and treated with 0.2 ml acetic anhydride for 7 hours at room temperature and for 14 hours in a refrigerator. It was then diluted with 1 ml of water, stirred for 10 min in an ice bath, diluted with 5 ml water and 20 ml ethyl acetate. The organic layer was washed with 3×5 ml of water, then with 5 ml saturated sodium bicarbonate, then with brine, dried over sodium sulfate and evaporated. The oily residue was taken up in 1:6 ethyl acetate-hexane, then flash chromatographed on a 13.5×110 mm column using 1:6 ethyl acetate-hexane as mobile phase for fractions 1-5, 1:4 ethyl acetate-hexane for the remaining fractions. Fractions 11-14 were pooled and evaporated to give 0.0184 g of the title compound (2).

### IV. Biological Examples and Formulation Examples

### Example 47

### Synergistic Effect of Calcitriol and Doxorubicin Inhibiting Bladder Cancer Cell Line Proliferation

Calcitriol has significant anti-proliferative effects on various tumor cell lines, but its clinical use as anti-cancer agent is limited by hypercalcemic liability. Superficial bladder cancer offers the unique advantage of being treatable by intravesical administration of anti-proliferative agents, thus permitting the use of high calcitriol concentrations while avoiding systemic side effects such as hypercalcemia and down-modulation of the anti-tumor immune response. Since studies in animal models have shown only marginal effects of intravesical calcitriol administration on superficial bladder cancer, we have examined the possibility of inducing synergistic effects by a combination of calcitriol and clinically used anti-proliferative agents. First, we tested in vitro several human bladder cancer cell lines for VDR expression and CYP24 upregulation following calcitriol treatment. Then, we tested inhibition of cell proliferation by calcitriol in combination with mitomycin c, cisplatin, gemcitabine, carboplatin, epirubicin or doxorubicin, and analyzed synergism or antagonism using the combination index method. Combination of calcitriol with mitomycin c, carboplatin or cisplatin did not result in any additive or synergistic effect, while an antagonistic effect was induced by combination with gemcitabine. Conversely, a clear-cut synergism was induced by calcitriol and doxorubicin and calcitriol and epilubicin in the majority of cell lines tested. Doxorubicin a standard agent administered intravesically in the treatment and recurrence prevention of superficial bladder cancer. It is well tolerated in most patients but efficacy, in particular the capacity to prevent tumor recurrence, is scarce. Epirubicin is also known the treatment and recurrence prevention of superficial bladder cancer. Based on our results, intravesical administration of calcitriol and doxorubicin or calcitriol and epirubicin could represent a promising treatment in superficial bladder cancer patients. Due to the synergistic interaction between the vitamin D compound (calcitriol) and the other anti-proliferative agent (doxorubicin or epirubicin) it may be possible to achieve greater efficacy of treatment for a given dose of anti-proliferative agent or to achieve a similar efficacy of treatment for a reduced dose of anti-proliferative agent (thereby reducing the toxic load on and potential carcinogenic liability to the patient).

### Materials and Methods

The following bladder cancer cell lines were used:

T24 (ECV)-Human bladder, transitional cell carcinoma—Grade 3
RT112/84-Human bladder carcinoma epithelial—Grade 1
HT 1.197-Human Caucasian bladder carcinoma—Grade 4 transitional carcinoma 44 years old male, no chemio or radiation therapy
HT 1376-Human Caucasian bladder carcinoma—Grade 3 transitional carcinoma 58 years old female no chemo or radiation therapy
RT4-Human Caucasian bladder transitional-cell carcinoma—Grade 1-2; explants of a recurring papillary tumor of the bladder

Bladder cancer cell lines were cultured in 96-well plates and calcitriol was added to the wells 24 h after plating. Proliferation was measured after an additional 96 h using CyQuant cell proliferation assay kit.

Drug interactions (as shown in FIG. 1) were evaluated by the median-effect/isobologram method using CalcuSyn software (Biosoft, Cambridge, UK). Combination Indices (CI) were calculated using the non-constant ratio combination design, determining synergism or antagonism for each data point.

FIG. 2 shows combination index values, for combined treatments with calcitriol and chemotherapeutic agents in the in vitro inhibition of human bladder cancer cell line proliferation. Recommended symbols are used for describing synergism or antagonism in drug combination studies analyzed with the combination index method (according to Chou. and Talalay, Adv. Enzyme Regul. 22 (1984) 27-55):

| Range of Combination | | |
|---|---|---|
| Index (CI) | Symbol | Description |
| <0.1 | +++++ | Very strong synergism |
| 0.1-0.3 | ++++ | Strong synergism |
| 0.3-0.7 | +++ | Synergism |
| 0.7-0.85 | ++ | Moderate synergism |
| 0.85-0.90 | + | Slight synergism |
| 0.90-1.10 | +/− | Nearly additive |
| 1.10-1.20 | − | Slight antagonism |
| 1.20-1.45 | −− | Moderate antagonism |
| 1.45-3.3 | −−− | Antagonism |
| 3.3-10 | −−−− | Strong antagonism |
| >10 | −−−−− | Very strong antagonism |

In the observation of constitutive expression of VDR in human bladder cancer cell lines as shown in FIG. 3, the indicated cell lines were cultured for 2 h in the presence of 1,25(OH)2vitaminD3 (=calcitriol) (100 nM) and the expression of 24-hydroxylase (CYP24) was analyzed by Real Time RT-PCR

In the observation of inhibition of bladder cancer cell line proliferation by calcitriol as shown in FIG. 4, bladder cancer cell lines were cultured in 96-well plates and calcitriol was added to the wells 24 h after plating. Proliferation was measured after an additional 96 h using CyQuant cell proliferation assay kit.

### Results

Human bladder cancer cell lines express VDR and its ligation leads to up-regulation of CYP24. Calcitriol inhibits bladder cancer cell line proliferation at mM range. Combination of calcitriol with mitomycin-c, cisplatin or carboplatin does not result in any additive or synergistic effect. A strong antagonistic effect is induced by the combination of calcitriol with gemcitabine. A strong synergistic effect is observed when doxorubicin and epirubicin are each used in combination with calcitriol.

### Example 48

### Sterile Solution for Intravesical Instillation

A sterile solution for intravesical instillation intended for intravesical administration in the urinary bladder is supplied as a nonaqueous solution that should be diluted before intravesical administration. Each 5 mL vial (200 mg valrubicin/150 μg calcitriol 5 mL vial) vial contains valrubicin at a concentration of 40 mg/mL and calcitriol at a concentration of 30 μg/mL in 50% CremophorEL (polyoxyethyleneglycol triricinoleate)/50% dehydrated alcohol, USP without preservatives or other additives. The solution is sterile and nonpyrogenic.

A dose of 800 mg valrubicin and 600 μg calcitriol is administered intravesically once a week for six weeks. Administration should be delayed at least two weeks after transurethral resection and/or fulguration. For each instillation, four vials of the formulation are allowed to warm slowly to room temperature, but are not heated. Twenty milliliters of formulation is then withdrawn from the four vials and diluted with 55 mL 0.9% Sodium Chloride Injection, USP to provide 75 mL of a diluted formulation solution. A urethral catheter is then inserted into the patient's bladder under aseptic conditions, the bladder drained, and the diluted 75 mL formulation solution instilled slowly via gravity flow over a period of several minutes. The catheter is then withdrawn. The patient should retain the formulation for two hours before voiding. At the end of two hours, all patients should void. (Some patients will be unable to retain the formulation for the full two hours.) Patients should be instructed to maintain adequate hydration following treatment.

### Example 49

### Evaluation of Combination Treatment with a VDR Agonist and an Anthracycline in an In Vivo Model and Measurement of Potential Local Toxicity of Such Combinations on Normal Mouse Bladder Mucosa

The following method demonstrates the evaluation of potential local toxicity of both a VDR antagonist (for example calcitriol) and an anthracycline (for example epirubicin) on normal mouse bladder mucosa, in absence of the tumor.

The model to be used is derived using mouse bladder tumor (MBT-2) obtained from transitional cell carcinoma of the bladder induced by oral administration of a carcinogen, and then maintained in vivo by subcutaneous growth. A single cell suspension is obtained from fresh tumor. Then, implantation in C3H mice is carried out via intravesical instillation of approx 5 million cells in 0.1 ml saline with 1 h contact time. The read-out from this model is the bladder weight and the histology. The timeframe for the model is approximately two weeks.

### Experimental Groups:

A) Single intravesical instillation of calcitriol, 0.3 ug/kg, 6 mice
B) Single intravesical instillation of calcitriol (0.3 ug/kg)+epirubicin, 100 ug/mouse, 6 mice
C) Six intravesical instillations (every other day) of calcitriol (0.3 ug/kg)+epirubicin (100 ug/mouse), 8 mice.

Animals are sacrificed 24 h after the last instillation in each experimental group. Serum and bladders are collected in order to measure calcemia, to measure bladder weight, and to perform histopathology on sections of paraffin-embedded bladders, evaluating any possible sign of local toxicity measured as mucosal irritation or urothelial damage, Three 3 untreated animals per group are used as controls.

Next, the treatment efficacy of combination therapy using VDR agonist and an anthracycline in tumor-bearing mice is measured by the following method:

Mouse bladder cancer cells are injected into the bladders of healthy mice before initiation of treatment. Intravesical instillation treatments are carried out in 0.1 ml and are repeated 6 times every other day on animals under anaesthesia. At the end of the experiment, the mice are sacrificed and bladders are harvested and weighed to analyse effects of therapy on bladder tumor growth, while blood is withdrawn to collect sera. Sera are analysed for calcemia that is used as a surrogate marker to assess systemic bio-availability and toxicity of the vitamin D compound (e.g. calcitriol).

All of the bladders are histologically assessed for the presence of the uptaken tumour. Bladders of treated animals that do not show any presence of the cancer are not considered as complete remission but, rather, as lack of tumor implantation and therefore disregarded.

### Experimental Groups:

D) Instillation of calcitriol. (low dose) 0.06 ug/kg, 5 mice
E) Instillation of calcitriol (high dose) 0.3 ug/kg, 5 mice
F) Instillation of epirubicin (100 ug/mouse), 5 mice
G) Instillation of calcitriol (0.06 ug/kg)+epirubicin (100 ug/mouse), 5 mice
H) Instillation of calcitriol (0.3 ug/kg)+epirubicin (100 ug/mouse), 5 mice
I) Instillation of vehicle alone, 5 mice
K) controls, no treatment, 5 mice

The experiment is ideally repeated three times.

### Reagents:

Epirubicin HCl is provided in 2× stock solution (2 mg/ml, in saline), ready to be used at 50 ul/mouse. Calcitriol is provided at 6 ug/ml in single-use vials (20 ul/aliquot), dissolved in EtOH 100% and stored at −70° C. under nitrogen atmosphere to minimize potential stability problems of calcitriol solution. One example of a drug vehicle is: 0.9% saline (pH 6), 0.1% (w/v) Tween 20. After addition of calcitriol, there can be a suggested concentration of 1% EtOH in the final instillation solution.

Instillation solutions must be freshly prepared every day and, possibly, immediately before the procedure. Working solutions are discarded after use.

### Example Instillation Solution Preparation:

| Number of instillations: | |
|---|---|
| 1 | 20 |

| Calcitriol dose | High | Low | High | Low |
|---|---|---|---|---|
| Final Vol (ul) | 100 | 100 | 2000 | 2000 |
| Vol stock epirubicin (ul) | 50 | 50 | 1000 | 1000 |
| Vol stock calcitriol (ul) | 1 | 0.2 | 20 | 4 |
| Vol vehicle (ul) | 49 | 49 | 980 | 980 |
| Vol 100% EtOH (ul) | 0 | 0.8 | 0 | 16 |
| Final % EtOH | 1 | 1 | 1 | 1 |

### Example 50

### Stability of an Example Solution for Intravesical Instillation

The vitamin D compound and other anti-proliferative agent of the present invention may be present in the same solution for simultaneous combination therapy. The stability of such solutions was therefore assessed.

### Method

### Preparation of Samples

### 1) Epirubicin

Approximately 12 mg of freeze-dried Farimorubicina® powder (approx. 2 mg of epirubicin) were dissolved in water to produce a concentrated stock solution. After vortexing, the concentrated stock was diluted 1:2 using a isotonic saline containing Tween 20 at 0.1% (w/v) to provide Sample B. After further vortexing, Sample B was stored at 4° C. until use.

### 2) Calcitriol

Using amber glassware, 1 mg calcitriol was dissolved in 1 ml ethanol. The solution was mixed and stored at −20° C. until use.

100 μl of the concentrated stock solution was then taken and diluted with 900 μl of isotonic saline containing Tween 20 0.1% (w/v). After shaking the sample was stored at 4° C. until use.

3) Combination of Calcitriol/Epirubicin (Calcitriol 50 μg/ml+epirubicin 1 mg/ml)

Using amber glassware 300 μl of epirubicin concentrated stock solution with 300 μl of calcitriol solution. Following mixing by vortexing, the sample was stored at 5° C. and analysed using HPLC chromatography after preparation (t=0) and after 2.5 hr (t=150 min).

### HPLC Conditions

| | |
|---|---|
| Column: | Supelcosil LC-1.25 cm × 4.6 mm, 5 um |
| Mobile phase: | 17 vol methanol:29 vol acetonitrile:54 vol |
| | Solution A |
| | Solution A: 3.7 g/l sodium lauryl sulfate |
| | and 2.8% (v/v) dilute phosphoric |
| | acid (i.e. 115 g concentrated H₃PO₄ |
| | and 885 g H₂O) in water. |
| Flowrate: | 2.5 ml/min |
| Column temperature: | 35° C. |
| Inection volume: | 50 ul |
| Detection: | UV at 254 nm and 230 nm |
| Retention time epirubicin: | 2.9 min approx |
| Retention time calcitriol: | 6.5 min approx |

### Results

1) Epirubicin (1 mg/ml).

FIG. 5 shows the UV chromatograms from detection at wavelength of 230 nm and 254 nm respectively from Run 1.

| | | | Area | Area |
|---|---|---|---|---|
| | run | tᵣ (min) | (230 nm) | (254 nm) |
| Epirubicin | 1 | 2,982 | 30443828 | 23243121 |
| | 2 | 2,928 | 30511286 | 23243599 |
| | 3 | 2,865 | 30555666 | 23383366 |

2) Calcitriol (100 μg/ml).

FIG. 6 shows the UV chromatograms from detection at 230 nm and 254 nm respectively from Run 1.

3)

| | | | Area | Area |
|---|---|---|---|---|
| | run | tᵣ (min) | (230 nm) | (254 nm) |
| Calcitriol | 1 | 6,884 | 3402919 | 4981173 |
| | 2 | 6,509 | 3485146 | 5116113 |

Combination (Calcitriol 50 μg/ml + Epirubicin 1 mg/ml).
t = 0 min

FIG. 7 shows the UV chromatograms from detection at 230 nm and 254 nm respectively from Run 1.

| | | | Area | Area |
|---|---|---|---|---|
| | Run | tᵣ (min) | (230 nm) | (254 nm) |
| Epirubicin | 1 | 2,962 | 53425733 | 44443024 |
| Calcitriol | | 6,215 | 2068192 | 2710511 |
| Epirubicin | 2 | 2,974 | 53877070 | 44731107 |
| Calcitriol | | 6,208 | 1960823 | 2589663 |

t = 150 min

FIG. 8 shows the UV chromatograms from detection at 230 nm and 254 nm respectively from Run 1

| | | | Area | Area |
|---|---|---|---|---|
| | run | tᵣ (min) | (230 nm) | (254 nm) |
| Epirubicin | 1 | 2,919 | 52245312 | 43984488 |
| Calcitriol | | 6,079 | 2027619 | 2674857 |
| Epirubicin | 2 | 2,918 | 50817806 | 43221785 |
| Calcitriol | | 6,077 | 1954501 | 2572268 |

### CONCLUSION

The results show that a freshly prepared combination has a chromatogram which is comparable to the overlapping results from the two single components, with the peak area values matching the theoretical prediction for both components. After 2.5 hrs at 5° C. the chromatogram profile is substantially unmodified.

In light of this evidence, under these test conditions, the combination of epirubicin and calcitriol does not indicate any issues with compatibility.

### Example 51

### Testing of Various VDR Agonists for Ability to Inhibit the Proliferation of Human Bladder Cancer Cells

A structurally diverse set of Vitamin D analogues were tested for the Inhibition of cell proliferation in 4 human bladder cancer cell lines: T24, RT112, HT 1376 and RT4.

Bladder cancer cells were dispensed at 103/well in 96-well fiat-bottom plates in 0.1 ml of complete medium (RPMI 1640 culture medium supplemented with 10% FCS, Hyclone Laboratories, Logan, Utah, 2 mM L-glutamine, 50 mg/ml gentamicin, 1 mM sodium pyravate and 1% non-essential amino acids). The plates were incubate for 24 h, to allow cell adhesion, and 0.1 ml of serial dilutions of calcitriol or the vitamin D3 analogue to be tested were added to the wells. After an additional 96-h culture, the medium was removed and the plates frozen at −80° C. Cell proliferation was evaluated using CyQuant cell proliferation assay kit from Molecular Probes (Eugene, Oreg.).

The results are shown in Table 1 below.

TABLE 1

| | Bladder Cancer Cell Lines | | |
|---|---|---|---|
| Vitamin D3 Analogues | IC50 (μM) | | |
| Chemical name | T24 | RT112 | RT4 |
| 1,25-Dihydroxycholecalciferol (Calcitriol) | 54.6 | 19/28.7 | 45/26 |
| 1,25-Dihydroxy-21-(2R,3-dihydroxy-3-methyl-butyl)-20R- | 57.9 | 25 | 71 |
| cholecalciferol | | | |
| 1,3-Di-O-acetyl-1,25-dihydroxy-23-yne-cholecalciferol | 40 | 8 | 85 |
| 1,3-Di-O-acetyl-1,25-dihydroxy-16-ene-23-yne-cholecalciferol | 27.7 | 3 | 80 |
| 1,3-Di-O-acetyl-1,25-dihydroxy-16,23E-diene-diolecalciferol | 58.7 | 14 | 82 |
| 1,3-Di-O-acetyl-1,25-dihydroxy-16-ene-cholecalciferol | 59.9 | 18 | 75 |
| 1,3,25-Tri-O-acetyl-1,25-dihydroxy-16-ene-23-yne-26,27-hexafluoro- | >100 | 17 | 95 |
| cholecalciferol | | | |
| 1,25-Dihydroxy-21-(2R,3-dihydroxy-3-methyl-butyl)-20S- | 69.4 | 1 | 62 |
| cholecalciferol | | | |
| 1,25-Dihydroxy-21-(2R,3-dihydroxy-3-methyl-butyl)-20S-19-nor- | 99.2 | 44 | 72 |
| cholecalciferol | | | |
| 1,3-Di-O-acetyl-1,25-dihydroxy-16-ene-23-yne-26,27-hexafluoro- | >100 | 26 | 19 |
| cholecalciferol | | | |
| 1,3-Di-O-acetyl-1,25-dihydroxy-16,23E-diene-25R-26-trifluoro- | 76.6 | 2 | 16 |
| cholecalciferol | | | |
| 1,3-Di-O-acetyl-1,25-dihydroxy-16-ene-23-yne-26,27-hexafluoro-19- | 29 | 15 | 9 |
| nor-cholecalciferol | | | |
| 1,3,25-Tri-O-acetyl-1,25-dihydroxy-16-ene-23-yne-26,27-hexafluoro- | 32 | 13 | 7 |
| 19nor-cholecalciferol | | | |
| 1,3-Di-O-acetyl-1,25-dihydroxy-16-ene-19-nor-cholecalciferol | 27.9 | 1 | 8 |
| 1,3-Di-O-acetyl-1,25-dihydroxy-16-ene-23-yne-19-nor-cholecalciferol | 26.7 | 1 | 9 |
| 1,3-Di-O-acetyl-1,25-dihydroxy-20-cyclopropyl-23-yne-19-nor- | 21.5 | 18 | 7 |
| cholecalciferol | | | |
| 1,3,25-Tri-O-acetyl-1,25-dihydroxy-20-cyclopropyl-23-yne-26,27- | 63.5 | 15 | 5 |
| hexafluoro-19-nor-cholecalciferol | | | |
| 1,3-Di-O-acetyl-1,25-dihydroxy-20-cyclopropyl-23-yne-26,27- | 25.5 | 2/13 | 4/1.8 |
| hexafluoro-19-nor-cholecalciferol | | | |
| 1,3-Di-O-acetyl-1,25-dihydroxy-16,23Z-diene-26,27-hexafluoro-19- | 55 | 1 | 20 |
| nor-cholecalciferol | | | |
| 1,3-Di-O-acetyl-1,25-dihydroxy-20-cyclopropyl-23-yne- | 49.6 | 8 | 13 |
| cholecalciferol | | | |
| 1,3-Di-O-acetyl-1,25-dihydroxy-16-ene-24-keto-19-nor- | 31 | 6 | 17 |
| cholecalciferol | | | |
| 1,3-Di-O-acetyl-1,25-dihydroxy-20-cyclopropyl-23E-ene-26,27- | 24.3 | 7 | 9 |
| hexafluoro-19-nor-cholecalciferol | | | |
| 1,3-Di-O-acetyl-1,25-dihydroxy-20-cyclopropyl-23Z-ene-26,27- | >100 | 16 | 18 |
| hexafluoro-19-nor-cholecalciferol | | | |
| 1,3-Di-O-acetyl-1,25-dihydroxy-16-ene-23-yne-26,27-bishomo-19- | 81.7 | 7 | 9 |
| nor-cholecalciferol | | | |
| 1,3-Di-O-acetyl-1,25-dihydroxy-20-cyclopropyl-cholecalciferol | 52.1 | 9 | 10 |
| 1,25-Dihydroxy-16-ene-20-cyclopropyl-23-yne-26,27-hexafluoro-19- | | 22.7 | 5.2 |
| nor-cholecalciferol | | | |
| 1,25-Dihydroxy-16-ene-20-cyclopropyl-23-yne-26,27-hexafluoro- | | 13.8 | 1.7 |
| cholecalciferol | | | |
| 1α-Fluoro-25-hydroxy-16-ene-20-cyclopropyl-23-yne-26,27- | | 16.9 | 3.6 |
| hexafluoro-cholecalciferol | | | |
| 1,25-Dihydroxy-16,23E-diene-20-cyclopropyl-26,27-hexafluoro-19- | | 14.5 | 4.6 |
| nor-cholecalciferol | | | |
| 1,25-Dihydroxy-16,23E-diene-20-cyclopropyl-26,27-hexafluoro- | | 10.6 | 2.3 |
| cholecalciferol | | | |
| 1α-Fluoro-25-hydroxy-16,23E-diene-20-cyclopropyl-26,27- | | 17 | 3.6 |
| hexafluoro-cholecalciferol | | | |
| 1,25-Dihydroxy-16,23Z-diene-20-cyclopropyl-26,27-hexafluoro-19- | | 9.6 | 2.2 |
| nor-cholecalciferol | | | |
| 1,25-Dihydroxy-16,23Z-diene-20-cyclopropyl-26,27-hexafluoro- | | 15.5 | 3.3 |
| cholecalciferol | | | |
| 1α-Fluoro-25-hydroxy-16,23Z-diene-20-cyclopropyl-26,27- | | 21 | 3.1 |
| hexafluoro-cholecalciferol | | | |
| 1,25-Dihydroxy-20S-21(3-hydroxy-3-methyl-butyl)-24-keto-19-nor- | | >30 | 2.6 |
| cholecalciferol | | | |
| 1,25-Dihydroxy-20S-21(3-hydroxy-3-methyl-butyl)-24-keto- | | 25.5 | 8.3 |
| cholecalciferol | | | |
| 1,25-Dihydroxy-16-ene-20-cyclopropyl-19-nor-cholecalciferol | | >30 | 9.9 |
| 1,3-Di-O-acetyl-1,25-dihydroxy-16-ene-20-cyclopropyl-19-nor- | | 17.4 | 3 |
| cholecalciferol | | | |
| 1,25-Dihydroxy-16-ene-20-cyclopropyl-cholecalciferol | | 27.6 | 2 |
| 1,3-Di-O-acetyl-1,25-dihydroxy-16-ene-20-cyclopropyl- | | 21.5 | 2.8 |
| cholecalciferol | | | |
| 1α-fluoro-25-hydroxy-16,23E-diene-26,27-bishomo-20-epi- | 28 | 12 | 16 |
| cholecalciferol | | | |
| 1,25-Dihydroxy-21-16,23Z-diene-26,27-hexafluoro-19-nor- | 58.7 | 24 | 20 |
| cholecalciferol | | | |

All of the Vitamin D3 analogues tested above show some activity in the inhibition of proliferation of one or more of the cell lines tested, whilst some compounds having an JC50 below 20 μM for any given cell line are clearly superior to calcitriol. These findings support a possible use of other vitamin D3 analogues in conjunction with an anti-proliferative agent for the treatment of bladder cancer.

### INCORPORATION BY REFERENCE

The contents of all references (including literature references, issued patents, published patent applications, and co-pending patent applications) cited throughout this application are hereby expressly incorporated herein in their entireties by reference.

Those skilled in the art will recognize, or be able to ascertain using no more than routine experimentation, many equivalents of the specific embodiments of the invention described herein. Such equivalents are intended with be encompassed by the following claims.

1. (canceled)

2. A method of treating a patient with bladder cancer by administering to a patient in need thereof an effective amount of a Vitamin D compound in combination with one or more other anti-proliferative agents.

3. (canceled)

4. The method of claim 1, wherein at least one of said anti-proliferative agents is an anthracycline.

5. The use or method of claim 4, wherein said anthracycline is selected from the group consisting of doxorubicin, epirubicin, daunorubicin, aclarubicin, idarubicin, pirarubicin, annamycin, methoxymorpholinodoxorubicin, cyanomorpholinyl doxorubicin, valrubicin (N-trifluoroacetyladriamycin-14-valerate) and mitoxantrone.

6. The method of claim 5, wherein said anthracycline is selected from the group consisting of valrubicin, doxorubicin and epirubicin.

7. The method of claim 6, wherein said anthracycline is epirubicin.

8. The method of claim 1, wherein said vitamin D compound is calcitriol.

9. The method of claim 1, wherein said vitamin D compound is a compound of the formula: 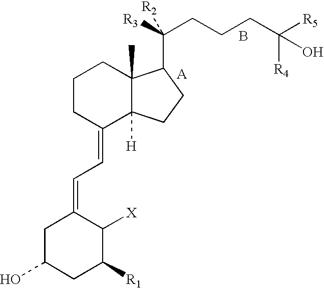
wherein
X is H₂ or CH₂
R, is hydrogen, hydroxy or fluorine
R₂ is hydrogen or methyl
R₃ is hydrogen or methyl. When R₂ or R₃ is methyl, R₃ or R₂ must be hydrogen.
R₄ is methyl, ethyl or trifluoromethyl
R₅ is methyl, ethyl or trifluoromethyl
A is a single or double bond
B is a single, E-double, Z-double or triple bond

10. The method of claim 9, wherein each of R₄ and R₅ is methyl or ethyl.

11. A kit containing a vitamin D compound together with instructions directing co-administration or sequential administration of the vitamin D compound with one or more other anti-proliferative agents to a patient in need of prevention or treatment of bladder cancer thereby to prevent or treat bladder cancer in said patient.

12. The kit according to claim 11 wherein the vitamin D compound and/or anti-proliferative agent is formulated in a pharmaceutical composition together with a pharmaceutically acceptable diluent or carrier.

13. A pharmaceutical composition comprising (i) a vitamin D compound; (ii) one or more antiproliferative agents and (iii) one or more pharmaceutically acceptable diluents or carriers.

14. The pharmaceutical composition according to claim 13 comprising (i) a vitamin D compound; (ii) one or more antiproliferative agents and (iii) one or more pharmaceutically acceptable diluents or carriers suitable for administration to the bladder by intravesical instillation.

15. (canceled)

16. A kit comprising (i) a pharmaceutical composition comprising a vitamin D compound together with one or more pharmaceutically acceptable diluents or carriers (ii) a pharmaceutical composition comprising one or more anti-proliferative agents together with one or more pharmaceutically acceptable diluents or carriers; and (iii) instructions directing co-administration or sequential administration of the compositions comprising the Vitamin D compound and the one or more anti-proliferative agents to a patient in need of prevention or treatment of bladder cancer thereby to prevent or treat bladder cancer in said patient.

17. The kit according to claim 11 wherein at least one of said anti-proliferative agents is an anthracycline.

18. The kit according to claim 17 wherein the anthracycline is epirubicin.

19. The kit according to claim 11 wherein said vitamin D compound is calcitriol.

20. The method according to claim 1 wherein the vitamin D compound and/or the one or more anti-proliferative agents are administered to the bladder by intravesical instillation.

21. The method according to claim 20 wherein the vitamin D compound and/or the one or more anti-proliferative agents are administered to the bladder by intravesical instillation.

22. The method according to claim 1 wherein the bladder cancer is superficial bladder cancer.

23. The pharmaceutical composition according to claim 13 wherein at least one of said anti-proliferative agents is an anthracycline.

24. The pharmaceutical composition according to claim 23 wherein the anthracycline is epirubicin.

25. The pharmaceutical composition according to claim 13 wherein said vitamin D compound is calcitriol.

26. The kit according to claim 16 wherein the vitamin D compound and/or the one or more anti-proliferative agents are administered to the bladder by intravesical instillation.

27. The kit according to claim 16 wherein the vitamin D compound and/or the one or more anti-proliferative agents are administered to the bladder by intravesical instillation.

28. The kit according to claim 16 wherein the bladder cancer is superficial bladder cancer.

29. The method according to claim 1 wherein the vitamin D compound and/or the one or more anti-proliferative agents are administered to the bladder by intravesical instillation.

30. The method according to claim 1 wherein the vitamin D compound and/or the one or more anti-proliferative agents are administered to the bladder by intravesical instillation.

31. The method according to claim 1 wherein the bladder cancer is superficial bladder cancer.
